(19) United States
(12) Patent Application Publication
(10) Pub. No.: US 2005/0232900 A1
(43) Pub. Date: Oct. 20, 2005

(54) Serotype of adenovirus and uses thereof

(75) Inventors: Ronald Vogels, Linschoten (NL); Angelique A.C. Lemckert, Voorschoten (NL); Menzo J.E. Havenga, Alphen a/d Rijn (NL)

Correspondence Address: TRASK BRITT, P.O. BOX 2550, SALT LAKE CITY, UT, 84110, US

(73) Assignee: Crucell Holland B.V., Leiden (NL)

(21) Appl. No.: 11/140,418

(22) Filed: May 27, 2005

Related U.S. Application Data
(63) Continuation-in-part of application No. 09/573,740, filed on May 18, 2000, now Pat. No. 6,913,922.
(63) Continuation-in-part of application No. 10/512,589, filed on Oct. 25, 2004.
(60) Provisional application No. 60/134,764, filed on May 18, 1999.

(30) Foreign Application Priority Data
Apr. 25, 2002 (WO) PCT/NL02/00280

(51) Int. Cl. A61K 48/00; C12N 7/00; C12N 15/861

(52) U.S. Cl. 424/93.2; 435/456; 435/235.1

(57) ABSTRACT

Adenovirus serotypes differ in their natural tropism. The adenovirus serotypes 2, 4, 5 and 7 all have a natural affiliation towards lung epithelia and other respiratory tissues. In contrast, serotypes 40 and 41 have a natural affiliation towards the gastrointestinal tract. The serotypes described differ in at least capsid proteins (penton-base, hexon), proteins responsible for cell binding (fiber protein), and proteins involved in adenovirus replication. This difference in tropism and capsid protein among serotypes has led to the many research efforts aimed at redirecting the adenovirus tropism by modification of the capsid proteins.

### CROSS-REFERENCE TO RELATED APPLICATIONS

This application is a continuation-in-part of U.S. patent application Ser. No. 09/537,740, filed May 18, 2000. This application is also a continuation-in-part of U.S. patent application Ser. No. 10/512,589, filed Oct. 25, 2004. Priority is claimed from International Patent Application Serial No. PCT/NL02/00280 filed Apr. 25, 2002. Under the provisions of 35 U.S.C. § 119(e), priority is claimed from U. S. Provisional Patent Application Ser. No. 60/134,764 filed May 18, 1999.

### STATEMENT ACCORDING TO 37 C.F.R. § 1.52(e)(5)-SEQUENCE LISTING SUBMITTED ON COMPACT DISC

Pursuant to 37 C.F.R. § 1.52(e)(1)(iii), a compact disc containing an electronic version of the Sequence Listing has been submitted concomitant with this application, the contents of which are hereby incorporated by reference. A second compact disc is submitted and is an identical copy of the first compact disc. The discs are labeled “copy 1” and “copy 2,” respectively, and each disc contains one file entitled “Sequence Listing as submitted.txt” which is 114 KB and created on May 20, 2005.

### TECHNICAL FIELD

The present invention relates generally to the field of biotechnology and gene delivery, particularly to gene therapy involving elements derived from viruses, more in particular, elements of adenoviruses. The invention also relates to the field of vaccination using recombinant adenoviruses.

### BACKGROUND

Adenoviruses have been proposed as suitable vehicles to deliver genes to a host. There are a number of features of adenoviruses that make them particularly useful for the development of gene-transfer vectors for human gene therapy:

The adenovirus genome is well characterized. It consists of a linear double-stranded DNA molecule of approximately 36,000 base pairs (“bp”). The adenovirus DNA contains identical Inverted Terminal Repeats (“ITRs”) of approximately 90 to 140 base pairs with the exact length depending on the serotype. The viral origins of replication are within the ITRs exactly at the genome ends.

The biology of the adenoviruses has been characterized in detail. The adenovirus is generally not associated with severe human pathology in immunocompetent individuals. The virus is extremely efficient in introducing its DNA into a host cell; the virus can infect a wide variety of cells and has a broad host range. The virus can be produced at high virus titers in large quantities.

The virus can be rendered replication defective by deletion of the early-region 1 (E1) of the viral genome (Brody et al., 1994). Most adenoviral vectors currently used in gene therapy have a deletion in the E1 region, where desired genetic information can be substituted.

Based on these features, preferred methods for in vivo gene transfer into human target cells make use of adenoviral vectors as gene delivery vehicles. However, drawbacks associated with the therapeutic use of adenoviral vectors in humans still exist. A major drawback is the existence of widespread pre-existing immunity among the population against adenoviruses. Exposure to wild-type adenoviruses is very common in humans, as has been documented extensively (reviewed in Wadell, 1984). This exposure has resulted in immune responses against most types of adenoviruses, not only against adenoviruses to which individuals have actually been exposed, but also against adenoviruses which have similar (neutralizing) epitopes. This phenomenon of pre-existing antibodies in humans, in combination with a strong secondary humoral and cellular immune response against the virus, can seriously affect gene transfer using recombinant adenoviral vectors.

To date, six different subgroups of human adenoviruses have been proposed which in total encompasses 51 distinct adenovirus serotypes (see Table 1). A serotype is defined on the basis of its immunological distinctiveness as determined by quantitative neutralization with animal antisera (horse, rabbit). If neutralization shows a certain degree of cross-reaction between two viruses, distinctiveness of serotype is assumed if A) the hemagglutinins are unrelated, as shown by lack of cross-reaction on hemagglutination-inhibition, or B) substantial biophysical/biochemical differences in DNA exist (Francki et al., 1991). The nine serotypes identified last (42 to 51) were isolated for the first time from HIV-infected patients (Hierholzer et al., 1988; Schnurr et al., 1993). For reasons not well understood, most of such immune-compromised patients shed adenoviruses that were rarely or never isolated from immune-competent individuals (Hierholzer et al., 1988, 1992; Khoo et al., 1995; De Jong et al., 1998).

The vast majority of people has had previous exposure to adenoviruses, especially the well-investigated adenovirus serotypes 5 and type 2 (“Ad5” and “Ad2”) or immunologically related serotypes. Importantly, these two serotypes are also the most extensively studied for use in human gene therapy.

As previously stated, the usefulness of these adenoviruses or cross-immunizing adenoviruses to prepare gene delivery vehicles may be seriously hampered, since the individual to which the gene delivery vehicle is provided will raise a neutralizing response to such a vehicle before long.

Thus, a need exists in the field of gene therapy to provide gene delivery vehicles, preferably based on adenoviruses, which do not encounter pre-existing immunity and/or which are capable of avoiding or diminishing neutralizing antibody responses.

### SUMMARY OF THE INVENTION

Thus, the invention provides a gene delivery vehicle comprising at least one Ad35 element or a functional equivalent thereof, responsible for avoiding or diminishing neutralizing activity against adenoviral elements by the host to which the gene is to be delivered and a gene of interest. A functional equivalent/homologue of an Ad35 (element) for the purposes of the present invention is an adenovirus (element) which, like adenovirus 35, encounters pre-existing immunity in less than about 10% of the hosts to which it is administered for the first time or which is capable in more than about 90% of the hosts to which it is administered to avoid or diminish the immune response.

Throughout the world, populations of humans can have varying pre-existing immunity profiles. For the present invention, the gene delivery vehicle of choice is preferably matched with a pre-existing immunity profile for the particular population in that geographic area. Typical examples of such adenoviruses are adenovirus serotypes 34, 26, 48 and 49. The invention also relates, therefore, to recombinant adenoviruses based on the human adenovirus serotypes Ad11, Ad26, Ad34, Ad35, Ad48 and, in particular, Ad49.

A gene delivery vehicle may be based on Ad35 or a functional homologue thereof, but it may also be based on another backbone, such as that of adenovirus 2 or 5, so long as it comprises at least one of the elements from Ad35 or a functional equivalent thereof, which leads to a diminishment of the immune response against such an Ad2- or Ad5-based gene delivery vehicle. Of course, the gene delivery vehicle may also comprise elements from other (adeno) viruses, so long as one replaces an element that could lead to immunity against such a gene delivery vehicle by an element of Ad35 or a functional homologue thereof, which has less of such a drawback and which, preferably, avoids such a drawback. The preferred functional homologue of Ad35 according to the present invention is Ad49. The invention, therefore, relates to recombinant replication-defective adenoviruses based on Ad49. Preferably, the recombinant virus comprises a gene of interest operably linked to a promoter. More preferably, the recombinant adenovirus based on Ad49 comprises a nucleic acid encoding the recombinant adenovirus. For the production of a subgroup D adenovirus such as Ad49 on Ad5-E1 immortalized packaging cells, it is preferred that the genomic nucleic acid further comprises the E4-Orf6 region of Ad5, thereby enabling a proper replication and production on such packaging cells.

In the present invention, a “gene delivery vehicle” is any vehicle capable of delivering a nucleic acid of interest to a host cell. It must, according to the invention, comprise an element of Ad35 or a functional equivalent thereof, which must have a beneficial effect regarding the immune response against such a vehicle. Basically, all other elements making up the vehicle can be any elements known in the art or developed in the art, as long as together they are capable of delivering the nucleic acid of interest. In principle, the person skilled in the art can use and/or produce any adenoviral products or production systems that can be, or have been, applied in the adenoviral field. Typically, the products of the invention can be made in the packaging cells useable with, for example, Ad5. The vectors based on Ad35 can typically be produced and/or used in the same manner as those of other adenoviruses, for example, Ad2 and/or Ad5.

A good overview of the possibilities of minimal vectors, packaging systems, intracellular amplification, vector and plasmid-based systems can be found in co-pending, co-owned International Patent Application PCT/NL99/00235 or U.S. Pat. No. 5,994,128 to Bout et al., incorporated herein by reference. Non-viral delivery systems can also be provided with elements according to the invention, as can viral delivery systems. Both kinds of systems are well known in the art in many different set-ups and do not, therefore, need any further elaboration here. A review on the many different systems and their properties can be found in Robbins and Ghivizzani (1998) and in Prince (1998), also incorporated herein by reference.

Gene delivery vehicles typically contain a nucleic acid of interest. A nucleic acid of interest can be a gene, or a functional part of a gene (wherein a gene is any nucleic acid which can be expressed), or a precursor of a gene, or a transcribed gene on any nucleic acid level (DNA and/or RNA: double- or single-stranded). Genes of interest are well known in the art and typically include those encoding therapeutic proteins such as TPA, EPO, cytokines, antibodies or derivatives thereof, etc.

An overview of therapeutic proteins to be applied in gene therapy is listed hereinafter. They include: immune-stimulatory factors like tumor-specific antigens, cytokines, etc.; anti-angiogenic factors non-limiting examples endostatin, angiostatin, ATF-BPTI CDT-6, dominant negative VEGF-mutants, etc.; angiogenic factors non-limiting example VEGF, fibroblast growth factors, nitric oxide synthases, C-type natriuretic peptide, etc.; inflammation-inhibiting proteins like soluble CD40, FasL, IL-12, IL-10, IL-4, IL-13 and excreted single-chain antibodies to CD4, CD5, CD7, CD52, Il-2, IL-1, IL-6, TNF, etc., or excreted single chain antibodies to the T-cell receptor on the auto-reactive T-cells. Also, dominant negative mutants of PML may be used to inhibit the immune response.

Furthermore, antagonists of inflammation-promoting cytokines may be used, for example, IL-1RA (receptor antagonist) and soluble receptors like sIL-1RI, sIL-1RII, sTNFRI and sTNFRII. Growth and/or immune response-inhibiting genes such as ceNOS, Bcl3, cactus and IκBα, β or γ and apoptosis-inducing proteins like the VP3 protein of chicken anemia virus may also be used. Furthermore, suicide genes like HSV-TK, cytosine deaminase, nitroreductase and linamerase may be used.

A nucleic acid of interest may also be a nucleic acid that can hybridize with a nucleic acid sequence present in the host cell, thereby inhibiting expression or transcription or translation of the nucleic acid. It may also block through co-suppression. In short, a “nucleic acid of interest” is any nucleic acid that one may wish to provide a cell with in order to induce a response by that cell, such as production of a protein, inhibition of such production, apoptosis, necrosis, proliferation, differentiation, etc.

We disclose adenovirus 35 (or a functional homologue thereof) for therapeutic use. The invention also provides an Ad35 or a functional homologue thereof or a chimeric virus derived therefrom, or a gene delivery vehicle based on the virus, its homologue, or its chimera, for use as a pharmaceutical. The serotype of the present invention, adenovirus type 35, is in itself known in the art. It is an uncommon group B adenovirus that was isolated from patients with acquired immunodeficiency syndrome and other immunodeficiency disorders (Flomenberg et al., 1987; De Jong et al., 1983). Ad35 has been shown to differ from the more fully characterized subgroup C (including Ad2 and Ad5) with respect to pathogenic properties (Basler et al., 1996). It has been suggested that this difference may be correlated with differences in the E3 region of the Ad35 genome (Basler et al., 1996). The DNA of Ad35 has been partially cloned and mapped (Kang et al., 1989a and b; Valderrama-Leon et al., 1985).

B-type adenovirus serotypes such as 34 and 35 have a different E3 region than other serotypes. Typically, this region is involved in suppressing immune response to adenoviral products. Thus, in one embodiment, the invention provides a gene delivery vehicle according to the invention wherein the elements involved in avoiding or diminishing immune response comprise Ad35 E3 expression products or the genes encoding them or functional equivalents of either or both.

Another part of adenoviruses involved in immune responses is the capsid, in particular, the penton and/or the hexon proteins. Thus, the invention also provides a gene delivery vehicle according to the invention, whereby the elements comprise at least one Ad35-capsid protein or functional part thereof, such as fiber, penton and/or hexon proteins or a gene encoding at least one of them. It is not necessary that a whole protein relevant for immune response be of Ad35 (or a functional homologue thereof) origin. It is very well possible to insert a part of an adenovirus fiber, penton or hexon protein into another fiber, penton or hexon. Thus, chimeric proteins are obtained.

It is also possible to have a penton of a certain adenovirus, a hexon from another, and a fiber or an E3 region from yet another adenovirus. According to the invention, at least one of the proteins or genes encoding them should comprise an element from Ad35 or a functional homologue thereof, whereby the element has an effect on the immune response of the host. Thus, the invention provides a gene delivery according to the invention, which is a chimera of Ad35 with at least one other adenovirus. In this way, one can also modify the resulting virus in aspects other than the immune response alone. One can enhance its efficiency of infection with elements responsible therefor; one can enhance its replication on a packaging cell or one can change its tropism.

Thus, the invention, for example, provides a gene delivery vehicle according to the invention that has a different tropism than Ad35. Of course, the tropism should be altered, preferably such that the gene delivery vehicle is delivered preferentially to a subset of the host's cells, i.e., the target cells. Changes in tropism and other changes that can also be applied in the present invention of adenoviral or other gene delivery vehicles are disclosed in co-pending, co-owned European Patent applications Nos. 98204482.8, 99200624.7 and 98202297.2, incorporated herein by reference. Of course, the present application also provides any and all building blocks necessary and/or useful to get to the gene delivery vehicles and/or the chimaeras, etc., of the present invention. This includes packaging cells such as PER.C6 (ECACC deposit number 96022940) or cells based thereon, but adapted for Ad35 or a functional homologue thereof. It also includes any nucleic acids encoding functional parts of Ad35 or a functional homologue thereof, such as helper constructs and packaging constructs, as well as vectors comprising genes of interest and, for example, an ITR, etc. Typically, the previously incorporated U.S. Pat. No. 5,994,128 to Bout et al. (Nov. 30, 1999), discloses elements necessary and useful for arriving at the invented gene delivery vehicles. Thus, the invention also provides a nucleic acid encoding at least a functional part of a gene delivery vehicle according to the invention, or a virus, homologue or chimera thereof according to the invention. According to the invention, such elements, which encode functions that will end up in the resulting gene delivery vehicle, must comprise or be encoded by a nucleic acid encoding at least one of the Ad35 elements or a functional equivalent thereof, responsible for avoiding or diminishing neutralizing activity against adenoviral elements by the host to which the gene is to be delivered. Typically, the gene of interest would be present on the same nucleic acid that means that such a nucleic acid has such a gene or that it has a site for introducing a gene of interest therein.

Typically, such a nucleic acid also comprises at least one ITR and, if it is a nucleic acid to be packaged, also a packaging signal. However, as mentioned before, all necessary and useful elements and/or building blocks for the present invention can be found in the incorporated U.S. Pat. No. 5,994,128 to Bout et al. A set of further improvements in the field of producing adenoviral gene delivery vehicles is applicant's plasmid system disclosed in PCT/NL99/00235 mentioned hereinbefore. This system works in one embodiment as a homologous recombination of an adapter plasmid and a longer plasmid, together comprising all elements of the nucleic acid to be incorporated in the gene delivery vehicle. These methods can also be applied to the presently invented gene delivery vehicles and their building elements. Thus, the invention also provides a nucleic acid according to the invention further comprising a region of nucleotides designed or useable for homologous recombination, preferably as part of at least one set of two nucleic acids comprising a nucleic acid according to the invention, whereby the set of nucleic acids is capable of a single homologous recombination event with each other, which leads to a nucleic acid encoding a functional gene delivery vehicle.

Both empty packaging cells (in which the vector to be packaged to make a gene delivery vehicle according to the invention still has to be introduced or produced), as well as cells comprising a vector according to the invention to be packaged, are provided. Thus, the invention also encompasses a cell comprising a nucleic acid according to the invention or a set of nucleic acids according to the invention, preferably a cell which complements the necessary elements for adenoviral replication that are absent from the nucleic acid according to the invention to be packaged, or from a set of nucleic acids according to the invention. In the present invention, it has been found that E1-deleted Ad35 vectors, are not capable of replication on cells that provide adenovirus 5 proteins in trans. The invention, therefore, further provides a cell capable of providing Ad35 E1 proteins in trans. Such a cell is typically a human cell derived from the retina or the kidney. Embryonic cells, such as amniocytes, have been shown to be particularly suited for the generation of an E1-complementing cell line. Such cells are, therefore, preferred in the present invention. Serotype-specific complementation by E1 proteins can be due to one or more protein(s) encoded by the E1 region. It is, therefore, essential that at least the serotype-specific protein be provided in trans in the complementing cell line. The non-serotype-specific E1 proteins essential for effective complementation of an E1-deleted adenovirus can be derived from other adenovirus serotypes. Preferably, at least an E1 protein from the E1B region of Ad35 is provided in trans to complement E1-deleted Ad35-based vectors. In one embodiment, nucleic acid encoding the one or more serotype-specific E1-proteins is introduced into the PER.C6 cell or a cell originating from a PER.C6 cell, or a similar packaging cell complementing with elements from Ad35 or a functional homologue thereof. As already alluded to, the invention also encompasses a method for producing a gene delivery vehicle according to the invention, comprising expressing a nucleic acid according to the invention in a cell according to the invention and harvesting the resulting gene delivery vehicle. The above refers to the filling of the empty packaging cell with the relevant nucleic acids. The format of the filled cell is, of course, also part of the present invention, which provides a method for producing a gene delivery vehicle according to the invention, comprising culturing a filled packaging cell (producer cell) according to the invention in a suitable culture medium and harvesting the resulting gene delivery vehicle.

The resulting gene delivery vehicles obtainable by any method according to the invention are, of course, also part of the present invention, particularly also a gene delivery vehicle that is derived from a chimera of an adenovirus and an integrating virus.

It is well known that adenoviral gene delivery vehicles do not normally integrate into the host genome. For long-term expression of genes in a host cell, it is, therefore, preferred to prepare chimaeras that do have that capability. Such chimaeras have been disclosed in co-pending, co-owned International Patent Application PCT/NL98/00731 incorporated herein by reference. A very good example of a chimera of an adenovirus and an integrating virus is where the integrating virus is an adeno-associated virus. As discussed hereinbefore, other useful chimaeras, which can also be combined with the above, are chimaeras (be it in swapping whole proteins or parts thereof, or both) that have altered tropism. A very good example thereof is a chimera of Ad35 and Ad16, possibly with elements from, for instance, Ad2 or Ad5, wherein the tropism-determining part of Ad16 or a functional equivalent thereof is used to direct the gene delivery vehicle to synoviocytes and/or smooth muscle cells (see co-pending, co-owned European patent applications Nos. 98204482.8 and 99200624.7) incorporated herein by reference). Dendritic cells (“DC”) and hemopoietic stem cells (“HSC”) are not easily transduced with Ad2- or Ad5-derived gene delivery vehicles. The present invention provides gene delivery vehicles that possess increased transduction capacity of DC and HSC cells. Such gene delivery vehicles at least comprise the tissue tropism-determining part of an Ad35 adenovirus. The invention, therefore, further provides the use of a tissue tropism-determining part of an Ad35 capsid for transducing dendritic cells and/or hemopoietic stem cells. Other B-type adenoviruses are also suited. A tissue tropism-determining part comprises at least the knob and/or the shaft of a fiber protein. Of course, it is very well possible for a person skilled in the art to determine the amino acid sequences responsible for the tissue tropism in the fiber protein. Such knowledge can be used to devise chimeric proteins comprising such amino acid sequences. Such chimeric proteins are, therefore, also part of the invention.

DC cells are very efficient antigen-presenting cells. By introducing the gene delivery vehicle into such cells, the host's immune system can be triggered toward specific antigens. Such antigens can be encoded by nucleic acid delivered to the DC or by the proteins of the gene delivery vehicle itself. The present invention, therefore, also provides a gene delivery vehicle with the capacity to evade the host immune system as a vaccine. The vector is capable of evading the immune system long enough to efficiently find its target cells and, at the same time, capable of delivering specific antigens to antigen-presenting cells, thereby allowing the induction and/or stimulation of an efficient immune response toward the specific antigen(s). To further modulate the immune response, the gene delivery vehicle may comprise proteins and/or nucleic acids encoding such proteins capable of modulating an immune response. Non-limiting examples of such proteins are found among the interleukins, adhesion molecules, co-stimulatory proteins, the interferons, etc. The invention, therefore, further provides a vaccine comprising a gene delivery vehicle of the invention. The invention further provides an adenovirus vector with the capacity to efficiently transduce DC and/or HSC, the vehicle comprising at least a tissue tropism-determining part of Ad35. The invention further provides the use of such delivery vehicles for the transduction of HSC and/or DC cells. Similar tissue tropisms are found among other adenoviruses of serotype B, particularly in Ad11 and are also part of the invention. Of course, it is also possible to provide other gene delivery vehicles with the tissue tropism-determining part, thereby providing such delivery vehicles with an enhanced DC- and/or HSC-transduction capacity. Such gene delivery vehicles are, therefore, also part of the invention.

The gene delivery vehicles according to the invention can be used to deliver genes or nucleic acids of interest to host cells. Such use will typically be a pharmaceutical one. Such a use is included in the present invention. Compositions suitable for such a use are also part of the present invention. The amount of gene delivery vehicle that needs to be present per dose or per infection (“m.o.i”) will depend on the condition to be treated, the route of administration (typically parenteral) the subject and the efficiency of infection, etc. Dose-finding studies are well known in the art and those already performed with other (adenoviral) gene delivery vehicles can typically be used as guides to find suitable doses of the gene delivery vehicles according to the invention. Typically, this is also where one can find suitable excipients, suitable means of administration, suitable means of preventing infection with the vehicle where it is not desired, etc. Thus, the invention also provides a pharmaceutical formulation comprising a gene delivery vehicle according to the invention and a suitable excipient, as well as a pharmaceutical formulation comprising an adenovirus, a chimera thereof, or a functional homologue thereof according to the invention and a suitable excipient.

### DESCRIPTION OF THE FIGURES

FIG. 1: Bar graph showing the percentage of serum samples positive for neutralization for each human wt adenovirus tested (see, Example 1 for description of the neutralization assay).

FIG. 2: Graph showing absence of correlation between the VP/CCID50 ratio and the percentage of neutralization.

FIG. 3: Schematic representation of a partial restriction map of Ad35 (taken from Kang et al., 1989) and the clones generated to make recombinant Ad35-based viruses.

FIG. 4: Bar graph presenting the percentage sera samples that show neutralizing activity to a selection of adenovirus serotypes. Sera were derived from healthy volunteers from Belgium and the UK.

FIG. 5: Bar graph presenting the percentage sera samples that show neutralizing activity to adenovirus serotypes 5, 11, 26, 34, 35, 48 and 49. Sera were derived from five different locations in Europe and the United States.

FIG. 6: Map of pAdApt.

FIG. 7: Map of pIPspAdapt.

FIG. 8: Map of pIPspAdapt1.

FIG. 9: Map of pIPspAdapt3.

FIG. 10: Map of pAdApt35IP3.

FIG. 11: Map of pAdApt35IP1.

FIG. 12: Schematic representation of the steps undertaken to construct pWE.Ad35.pIX-rITR.

FIG. 13: Map of pWE.Ad35.pIX-rITR.

FIG. 14: Map of pRSV.Ad35-E1.

FIG. 15: Map of PGKneopA.

FIG. 16: Map of pRSVpNeo.

FIG. 17: Map of pRSVhbvNeo.

FIG. 18: Flow cytometric analyses on GFP expression in human TF-1 cells. Non-transduced TF-1 cells were used to set a background level of 1%. GFP expression in cells transduced with Ad5, Ad5.Fib16, Ad5.Fib17, Ad5.Fib4-L, Ad5.Fib35, and Ad5.Fib51 is shown.

FIG. 19: Transduction of primary human fibroblast-like stroma. Cells were analyzed 48 hours after a two-hour exposure to the different chimeric fiber viruses. Shown is percentage of cells found positive for the transgene: GFP using a flow cytometer. Non-transduced stroma cells were used to set a background at 1%. Results of different experiments (n=3) are shown ±standard deviation.

FIG. 20: Transduction of primary human fibroblast-like stroma, CD34⁺ cells and CD34⁺Lin⁻ cells. Cells were analyzed five days after a two-hour exposure to the different chimeric fiber viruses. Shown is percentage of cells found positive for the transgene: GFP using a flow cytometer. Non-transduced cells were used to set a background at 1%. Also shown is the number of GFP-positive events divided by the total number of events analyzed (between brackets).

FIG. 21: (A) Flow cytometric analysis of GFP-positive cells after transduction of CD34⁺ cells with Ad5.Fib51. All cells gated in R2-R7 are positive for CD34 but differ in their expression of early differentiation markers CD33, CD38, and CD71 (Lin). Cells in R2 are negative for CD333, CD38, and CD71, whereas cells in R7 are positive for these markers. To demonstrate specificity of Ad5.Fib51, the percentage of GFP-positive cells was determined in R2-R7 that proved to decline from 91% (R2) to 15% (R7). (B) Identical experiment as shown -under (A) (X-axes is R2-R7) but for the other Ad fiber chimeric viruses showing that Ad5.Fib35 and Ad5.Fib16 behave similar as Ad5.Fib51.

FIG. 22: Alignment of the chimeric fiber proteins of Ad5fib16, Ad5fib35 and Ad5fib51 with the Ad5 fiber sequence.

FIG. 23: Toxicity of Adenovirus exposure to primitive human bone marrow cells and stem cells. Cell cultures were counted just before and five days after adenovirus transduction. Shown is the percentage of primitive human bone marrow cells (CD34⁺) and HSCs (CD34⁺Lin⁻) recovered as compared to day 0.

FIG. 24: Transduction of immature DCs at a virus dose of 100 or 1000 virus particles per cell. Virus tested is Ad5 and Ad5-based vectors carrying the fiber of serotype 12 (Ad5.Fib12), 16 (Ad5.Fib16), 28 (Ad5.Fib28), 32 (Ad5.Fib32), the long fiber of 40 (Ad5.Fib40-L), 49 (Ad5.Fib49), 51 (Ad5.Fib51). Luciferase transgene expression is expressed as relative light units per microgram of protein.

FIG. 25: Flow cytometric analyses of LacZ expression on immature and mature DCs transduced with 10,000 virus particles per cell of Ad5 or the fiber chimeric vectors Ad5.Fib16, Ad5.Fib40-L, or Ad5.Fib51. Percentages of cells scored positive are shown in upper left corner of each histogram.

FIG. 26: Luciferase transgene expression in human immature DCs measured 48 hours after transduction with 1,000 or 5,000 virus particles per cell. Viruses tested were fiber chimeric viruses carrying the fiber of subgroup B members (serotypes 11, 16, 35, and 51).

FIG. 27: GFP expression in immature human DCs 48 hours after transduction with 1,000 virus particles per cell of Ad5, Ad5.Fib16, and Ad5.Fib35. Non-transduced cells were used to set a background level of approximately 1% (−).

FIG. 28: Transduction of mouse and chimpanzee DCs. Luciferase transgene expression measured in mouse DCs 48 hours after transduction is expressed as relative light units per microgram of protein. Chimpanzee DCs were measured 48 hours after transduction using a flow cytometer. GFP expression demonstrates the poor transduction of Ad (35) in contrast to Ad5.Fib35 (66%).

FIG. 29: Temperature-dependent growth of PER.C6. PER.C6 cells were cultured in DMEM supplemented with 10% FBS (Gibco BRL) and 10 mM MgCl₂ in a 10% CO₂ atmosphere at 32° C., 37° C. or 39° C. At day 0, a total of 1×10⁶ PER.C6 cells were seeded per 25 cm² tissue culture flask (Nunc) and the cells were cultured at 32° C., 37° C. or 39° C. At day 1 through 8, cells were counted. The growth rate and the final cell density of the PER.C6 culture at 39° C. are comparable to that at 37° C. The growth rate and final density of the PER.C6 culture at 32° C. were slightly reduced as compared to that at 37° C. or 39° C. PER.C6 cells were seeded at a density of 1×10⁶ cells per 25 cm² tissue culture flask and cultured at 32° C., 37° C. or 39° C. At the indicated time points, cells were counted in a Burker cell counter. PER.C6 grows well at 32° C., 37° C. and 39° C.

FIG. 30: DBP levels in PER.C6 cells transfected with pcDNA3, pcDNA3wtE2A or pcDNA3ts125E2A. Equal amounts of whole-cell extract were fractionated by SDS-PAGE on 10% gels. Proteins were transferred onto Immobilon-P membranes and DBP protein was visualized using the αDBP monoclonal B6 in an ECL-detection system. All of the cell lines derived from the pcDNA3ts125E2A transfection express the 72-kDa E2A-encoded DBP protein (left panel: lanes 4 to 14; middle panel: lanes 1 to 13; right panel: lanes 1 to 12). In contrast, the only cell line derived from the pcDNAwtE2A transfection did not express the DBP protein (left panel, lane 2). No DBP protein was detected in extract from a cell line derived from the pcDNA3 transfection (left panel, lane 1), which serves as a negative control. Extract from PER.C6 cells transiently transfected with pcDNA3ts125 (left panel, lane 3) served as a positive control for the Western blot procedure. These data confirm that constitutive expression of wtE2A is toxic for cells and that using the ts125 mutant of E2A can circumvent this toxicity.

FIG. 31: Suspension growth of PER.C6ts125E2A C5-9. The tsE2A-expressing cell line PER.C6tsE2A.c5-9 was cultured in suspension in serum-free Ex-cell™. At the indicated time points, cells were counted in a Burker cell counter. The results of 8 independent cultures are indicated. PER.C6tsE2A grows well in suspension in serum-free Ex-cell™ medium.

FIG. 32: Growth curve PER.C6 and PER.C6tsE2A. PER.C6 cells or PER.C6ts125E2A (c8-4) cells were cultured at 37° C. or 39° C., respectively. At day 0, a total of 1×10⁶ cells were seeded per 25 cm² tissue culture flask. At the indicated time points, cells were counted. The growth of PER.C6 cells at 37° C. is comparable to the growth of PER.C6ts125E2A c8-4 at 39° C. This shows that constitutive over-expression of ts125E2A has no adverse effect on the growth of cells at the non-permissive temperature of 39° C.

FIG. 33: Stability of PER.C6ts125E2A. For several passages, the PER.C6ts125E2A cell line clone 8-4 was cultured at 39° C. in medium without G418. Equal amounts of whole-cell extract from different passage numbers were fractionated by SDS-PAGE on 10% gels. Proteins were transferred onto Immobilon-P membranes and DBP protein was visualized using the αDBP monoclonal B6 in an ECL-detection system. The expression of ts125E2A-encoded DBP is stable for at least 16 passages, which is equivalent to approximately 40 cell doublings. No decrease in DBP levels was observed during this culture period, indicating that the expression of ts125E2A is stable, even in the absence of G418 selection pressure.

FIG. 34: tTA activity in hygromycin resistant PER.C6/tTA (A) and PER/E2A/tTA (B) cells. Sixteen independent hygromycin-resistant PER.C6/tTA cell colonies and 23 independent hygromycin-resistant PER/E2A/tTA cell colonies were grown in 10 cm² wells to sub-confluency and transfected with 2 μg of pUHC 13-3 (a plasmid that contains the reporter gene luciferase under the control of the 7×tetO promoter). One half of the cultures were maintained in medium containing doxycycline to inhibit the activity of tTA. Cells were harvested at 48 hours after transfection and luciferase activity was measured. The luciferase activity is indicated in relative light units (RLU) per μg protein.

FIG. 35: Schematic representation of the Ad49 vector system in a three (A: double homologous recombination) and in a two (B: single homologous recombination) plasmid-based system. Numbers refer to the nucleotide numbering of the wt Ad49 genome sequence. Early regions (E) and late regions (L) are indicated. ITR=Inverted Terminal Repeat, ψ=packaging signal.

FIG. 36: Plasmid pAdApt49.

FIG. 37: Plasmid pBrAd49SfiI.

FIG. 38: Plasmid pBrAd49Srfl-rITR.

FIG. 39: Plasmid pBrAd49.Srf-rITRdE3.5orf6.

FIG. 40: SIV-gag-induced responses in the presence of low anti-Ad49 immunity. (A) Neutralizing antibody titers present in mice that were injected first with Ad49 wt virus and four weeks later with Ad5- or Ad35.SIV-gag vectors. (B) Percentage of T-cells reacting against a dominant SIV-Gag peptide in mice analyzed over time using tetramers. SIV-gag responses were measured in mice with and without pre-immunization with Ad49 virus.

FIG. 41: SIV-gag-induced responses in the presence of high anti-Ad49 immunity. (A) Titers of specific neutralizing antibodies against Ad49 viruses present in mice that were injected the first two times with Ad49 wt virus and four weeks later with Ad5- or Ad35.SIV-Gag vectors. (B) Percentage of T-cells reacting against a dominant SIV-Gag peptide in mice analyzed over time using tetramers. SIV-Gag responses were measured in mice with and without pre-immunization with Ad49 virus.

### DETAILED DESCRIPTION OF THE INVENTION

As previously stated, the most extensively studied serotypes of adenovirus are not ideally suited for delivering additional genetic material to host cells. This fact is partially due to the pre-existing immunity among the population against these serotypes. This presence of pre-existing antibodies in humans, in combination with a strong secondary humoral and cellular immune response against the virus, will affect adenoviral gene therapy.

The present invention provides the use of at least elements of a serotype and functional homologues thereof of adenovirus that are very suitable as gene therapy vectors. The present invention also discloses an automated high-throughput screening of all known adenovirus serotypes against sera from many individuals. Surprisingly, no neutralizing ability was found in any of the sera that were evaluated against one particular serotype, adenovirus 35 (“Ad35”). This makes the serotype of the present invention extremely useful as a vector system for gene therapy in man. Such a vector system is capable of efficiently transferring genetic material to a human cell without the inherent problem of pre-existing immunity.

Typically, a virus is produced using an adenoviral vector (typically a plasmid, cosmid, or baculovirus vector). Such vectors are, of course, also part of the present invention.

The invention also provides adenovirus-derived vectors that have been rendered replication defective by deletion or inactivation of the E1 region. Of course, a gene of interest can also be inserted at, for instance, the site of E1 of the original adenovirus from which the vector is derived.

In all aspects of the invention, the adenoviruses may contain deletions in the E1 region and insertions of heterologous genes, either linked or not linked to a promoter. Furthermore, the adenoviruses may contain deletions in the E2, E3 or E4 regions and insertions of heterologous genes linked to a promoter. In these cases, E2- and/or E4-complementing cell lines are used to generate recombinant adenoviruses.

One may choose to use the Ad35 serotype itself for the preparation of recombinant adenoviruses to be used in gene therapy. Alternatively, one may choose to use elements derived from the serotype of the present invention in such recombinant adenoviruses. One may, for instance, develop a chimeric adenovirus that combines desirable properties from different serotypes. Some serotypes have a somewhat limited host range but have the benefit of being less immunogenic, while others are the other way around. Some have a problem of being of a limited virulence but have a broad host range and/or a reduced immunogenicity. Such chimeric adenoviruses are known in the art and they are intended to be within the scope of the present invention. Thus, in one embodiment, the invention provides a chimeric adenovirus comprising at least a part of the adenovirus genome of the present serotype, providing it with absence of pre-existing immunity and at least a part of the adenovirus genome from another adenovirus serotype, resulting in a chimeric adenovirus. In this manner, the chimeric adenovirus produced is such that it combines the absence of pre-existing immunity of the serotype of the present invention to other characteristics of another serotype. Such characteristics may be temperature stability, assembly, anchoring, redirected infection, production yield, redirected or improved infection, stability of the DNA in the target cell, etc.

A packaging cell will generally be needed in order to produce a sufficient amount of adenoviruses. For the production of recombinant adenoviruses for gene therapy purposes, several cell lines are available. These include, but are not limited to, the known cell lines PER.C6, 911, 293, and E1 A549.

An important feature of the present invention is the means to produce the adenovirus. Typically, one does not want an adenovirus batch for clinical applications to contain replication-competent adenovirus. In general, therefore, it is desired to omit a number of genes (but at least one) from the adenoviral genome on the adenoviral vector and to supply these genes in the genome of the cell in which the vector is brought to produce chimeric adenovirus. Such a cell is usually called a “packaging cell.” The invention thus also provides a packaging cell for producing an adenovirus (a gene delivery vehicle) according to the invention, comprising in trans all elements necessary for adenovirus production not present on the adenoviral vector according to the invention. Typically, vector and packaging cells have to be adapted to one another so that they have all the necessary elements, but that they do not have overlapping elements, which lead to replication-competent virus by recombination.

Thus, the invention also provides a kit of parts comprising a packaging cell according to the invention and a recombinant vector according to the invention, wherein essentially no sequence overlap leading to recombination, resulting in the production of replication-competent adenovirus, exists between the cell and the vector.

Thus, the invention provides methods for producing adenovirus, which, upon application, will escape pre-existing humoral immunity. Such a method includes providing a vector with elements derived from an adenovirus serotype against which virtually no natural immunity exists and transfecting the vector in a packaging cell according to the invention and allowing for production of viral particles.

In one aspect, the invention includes the use of the adenovirus serotype of the present invention to overcome naturally existing or induced neutralizing host activity towards adenoviruses administered in vivo for therapeutic applications. The need for a new serotype is stressed by observations that 1) repeated systemic delivery of recombinant Ad5 is unsuccessful due to the formation of high titers of neutralizing antibodies against recombinant Ad5 (Schulick et al., 1997), and 2) pre-existing or humoral immunity is already widespread in the population.

In another aspect, the invention provides the use of gene delivery vehicles of the invention or the use of Ad35 for vaccination purposes. Such use prevents, at least in part, undesired immune responses of the host. Non-limiting examples of undesired immune responses include evoking an immune response against the gene delivery vehicle or Ad35 and/or boosting an immune response against the gene delivery vehicle or Ad35.

In another aspect of the invention, alternating use is made of Ad vectors belonging to different subgroups. This aspect of the invention, therefore, circumvents the inability to repeat the administration of an adenovirus for gene therapy purposes.

The invention further relates to the production of recombinant replication-defective adenoviruses based on adenovirus serotype Ad49 on packaging cells that have been immortalized by the E1 region of adenovirus, preferably Ad5. More preferably, PER.C6® cells or derivatives thereof are used for the production of the recombinant replication-defective viral vectors according to the invention. In WO 03/104467 and WO 2004/001032 (both incorporated by reference herein), it is outlined that the E4-Orf6 gene product of an adenovirus should be compatible with the E1B-55K gene product produced in the packaging cell to obtain a proper replication and growth of virus particles in the packaging cell. The PER.C6® cells have been immortalized with the E1 region (including E1B-55K) of Ad5, which is a subgroup C adenovirus. Ad49 is a subgroup D adenovirus, whereas Ad35 is a subgroup B adenovirus. Therefore, in concert of what has been described above for Ad35 and in the cited references, the production of Ad49 on Ad5-E1-immortalized cells requires a compatible E1B-55K/E4-Orf6 combination. In a preferred embodiment, the invention provides recombinant replication-defective adenoviral vectors based on adenovirus serotype Ad49, wherein the genomic nucleic acid of the adenoviral vector comprises an E4-Orf6 region from a subgroup C adenovirus, preferably Ad5. In a more preferred embodiment, the Ad49 E4-Orf6 region is removed and replaced by the E4-Orf6 region from Ad5. In this preferred aspect, the Ad49 vector is a chimeric adenovirus of Ad49 and Ad5. In the examples given below, it is explained that (part of) the E4-Orf6/7 region is also replaced. This however, is due to cloning purposes and not critical for the invention.

In a further embodiment, the invention also relates to new packaging cell lines that are able to sustain growth of subgroup D adenoviruses. Such cell lines may either be immortalized by the E1 region of a subgroup D adenovirus, preferably Ad49, be immortalized by a chimeric nucleic acid comprising E1A and E1B-21K from Ad5 and E1B-55K from a subgroup D adenovirus, preferably Ad49, or be immortalized by the E1 region of Ad5 and supplemented with the E1B-55K region of a subgroup D adenovirus, preferably Ad49.

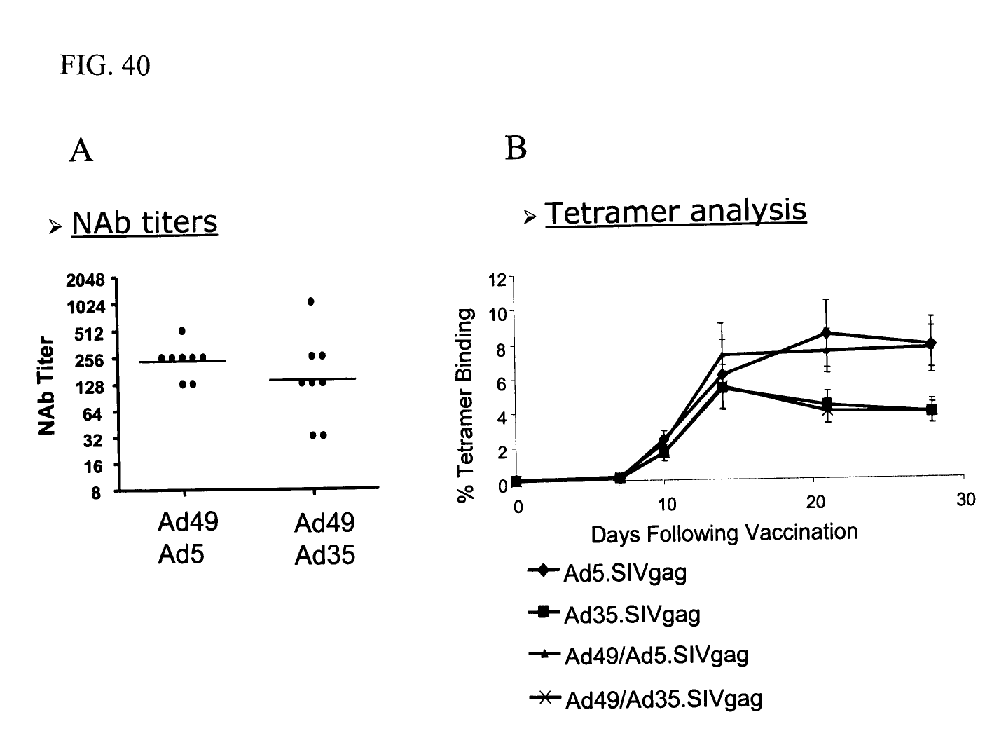

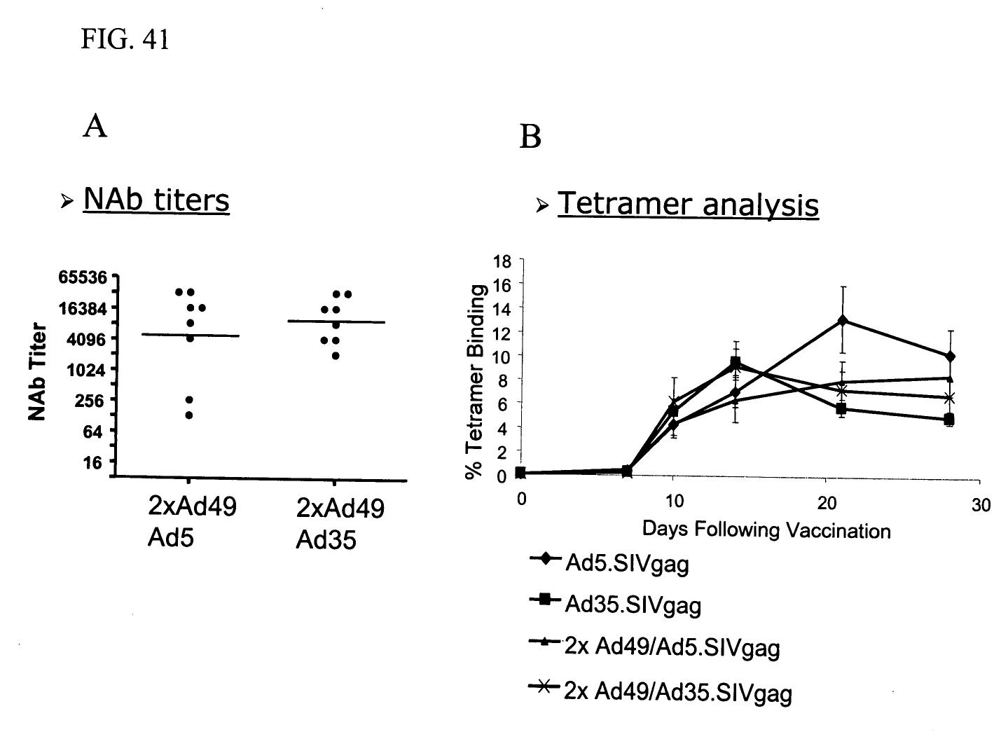

As explained in the examples, it is useful to use different adenoviral serotypes in a prime boost regimen when it is required to strengthen the immune response towards a given antigen. With this, it is preferred to use serotypes taken from different subgroups as the serotypes within one subgroup contain capsid proteins (fiber, penton, hexon) that have a homology that is too high that cross-neutralization may occur. It is shown in FIGS. 40 and 41 and Table V herein that Ad49 is a suitable vector that can be used in prime boost setups with other low-neutralized vectors from other subgroups, preferably subgroup B, more preferably, Ad11 and Ad35. Thus, the invention also relates to methods of inducing an immune response in a mammal in need of vaccination, wherein a priming composition is followed by a boosting composition, both compositions comprising a recombinant replication-defective adenovirus, and wherein the adenoviruses in the priming composition and boosting composition are from different subgroups. Preferably, the different subgroups are subgroup B and D. More preferably, the choice of serotype from subgroup B is Ad11 or Ad35, whereas the preferred choice from subgroup D is Ad49. The preferred serotypes are those that encounter low-neutralizing effect when introduced in the mammal.

The invention is further explained by the use of the following illustrative Examples.

### EXAMPLES

### Example 1

A High-Throughput Assay for the Detection of Neutralizing Activity in Human Serum

To enable screening of a large amount of human sera for the presence of neutralizing antibodies against all adenovirus serotypes, an automated 96-well assay was developed.

Human sera: A panel of 100 individuals was selected. Volunteers (50% male, 50% female) were healthy individuals between ages 20 and 60 years old with no restriction for race. All volunteers signed an informed consent form. People professionally involved in adenovirus research were excluded.

Approximately 60 ml blood was drawn in dry tubes. Within two hours after sampling, the blood was centrifuged at 2500 rpm for ten minutes. Approximately 30 ml serum was transferred to polypropylene tubes and stored frozen at −20° C. until further use.

Serum was thawed and heat-inactivated at 56° C. for ten minutes and then aliquoted to prevent repeated cycles of freeze/thawing. Part was used to make five steps of two-fold dilutions in medium (DMEM, Gibco BRL) in a quantity enough to fill out approximately 70 96-well plates. Aliquots of undiluted and diluted sera were pipetted in deep-well plates (96-well format) and using a programmed platemate, dispensed in 100 μl aliquots into 96-well plates. This way, the plates were loaded with eight different sera in duplo (100 μl/well) according to the scheme below:

| | | | | | | | | | | | |
|---|---|---|---|---|---|---|---|---|---|---|---|
| S1/2 | S1/4 | S1/8 | S1/16 | S1/32 | S5/2 | S5/4 | S5/8 | S5/16 | S5/32 | — | — |
| S1/2 | S1/4 | S1/8 | S1/16 | S1/32 | S5/2 | S5/4 | S5/8 | S5/16 | S5/32 | — | — |
| S2/2 | S2/4 | S2/8 | S2/16 | S2/32 | S6/2 | S6/4 | S6/8 | S6/16 | S6/32 | — | — |
| S2/2 | S2/4 | S2/8 | S2/16 | S2/32 | S6/2 | S6/4 | S6/8 | S6/16 | S6/32 | — | — |
| S3/2 | S3/4 | S3/8 | S3/16 | S3/32 | S7/2 | S7/4 | S7/8 | S7/16 | S7/32 | — | — |
| S3/2 | S3/4 | S3/8 | S3/16 | S3/32 | S7/2 | S7/4 | S7/8 | S7/16 | S7/32 | — | — |
| S4/2 | S4/4 | S3/8 | S3/16 | S3/32 | S8/2 | S8/4 | S8/8 | S8/16 | S8/32 | — | — |
| S4/2 | S4/4 | S3/8 | S3/16 | S3/32 | S8/2 | S8/4 | S8/8 | S8/16 | S8/32 | — | — |

Where S1/2 to S8/2 in columns 1 and 6 represent 1 × diluted sera and Sx/4, Sx/16 and Sx/32, the two-fold serial dilutions. The last plates also contained four wells filled with 100 μl fetal calf serum as a negative control.

Plates were kept at −20° C. until further use.

Preparation of Human Adenovirus Stocks

Prototypes of all known human adenoviruses were inoculated on T25 flasks seeded with PER.C6 cells (Fallaux et al., 1998) and harvested upon full CPE. After freeze/thawing, 1 to 2 ml of the crude lysates was used to inoculate a T80 flask with PER.C6 and the virus was harvested at full CPE. The timeframe between inoculation and occurrence of CPE, as well as the amount of virus needed to re-infect a new culture, differed between serotypes. Adenovirus stocks were prepared by freeze/thawing and used to inoculate 3 to 4 T175 cm² three-layer flasks with PER.C6 cells. Upon occurrence of CPE, cells were harvested by tapping the flask, pelleted and the virus was isolated and purified by a two-step CsCl gradient as follows. Cell pellets were dissolved in 50 ml 10 mM NaPO₄ buffer (pH 7.2) and frozen at −20° C. After thawing at 37° C., 5.6 ml sodium deoxycholate (5% w/v) was added. The solution was mixed gently and incubated for 5 to 15 minutes at 37° C. to completely lyse the cells. After homogenizing the solution, 1875 μl 1 M MgCl₂ was added. After the addition of 375 μl DNAse (10 mg/ml), the solution was incubated for 30 minutes at 37° C. Cell debris was removed by centrifugation at 1880×g for 30 minutes at RT without brake. The supernatant was subsequently purified from proteins by extraction with FREON (3×). The cleared supernatant was loaded on a 1 M Tris/HCl buffered cesium chloride block gradient (range: 1.2/1.4 g/ml) and centrifuged at 21,000 rpm for 2.5 hours at 10° C. The virus band is isolated after which a second purification using a 1 M Tris/HCl buffered continues gradient of 1.33 g/ml of cesium chloride was performed. The virus was then centrifuged for 17 hours at 55,000 rpm at 10° C. The virus band is isolated and sucrose (50% w/v) is added to a final concentration of 1%. Excess cesium chloride is removed by dialysis (three times one hour at RT) in dialysis slides (Slide-a-lizer, cut off 10,000 kDa, Pierce, USA) against 1.5 liter PBS supplemented with CaCl₂ (0.9 mM), MgCl₂ (0.5 mM) and an increasing concentration of sucrose (1, 2, 5%). After dialysis, the virus is removed from the slide-a-lizer after which it is aliquoted in portions of 25 and 100 μl upon which the virus is stored at −85° C.

To determine the number of virus particles per milliliter, 50 μl of the virus batch is run on a high-pressure liquid chromatograph (HPLC) as described by Shabram et al., 1997. Viruses were eluted using a NaCl gradient ranging from 0 to 600 mM. As depicted in Table I, the NaCl concentration by which the viruses were eluted differed significantly among serotypes.

Most human adenoviruses replicated well on PER.C6 cells with a few exceptions. Adenovirus type 8 and 40 were grown on 911-E4 cells (He et al., 1998). Purified stocks contained between 5×10¹⁰ and 5×10¹² virus particles/ml (VP/ml; see Table I).

Titration of Purified Human Adenovirus Stocks

Adenoviruses were titrated on PER.C6 cells to determine the amount of virus necessary to obtain full CPE in five days, the length of the neutralization assay. Hereto, 100 μl medium was dispensed into each well of 96-well plates. 25 μl of adenovirus stocks pre-diluted 10⁴, 10⁵, 10⁶ or 10⁷ times were added to column 2 of a 96-well plate and mixed by pipetting up and down ten times. Then 25 μl was brought from column 2 to column 3 and again mixed. This was repeated until column 11, after which 25 μl from column 11 was discarded. This way, serial dilutions in steps of five were obtained starting off from a pre-diluted stock. Then 3×10⁴ PER.C6 cells (ECACC deposit number 96022940) were added in a 100 μl volume and the plates were incubated at 37° C., 5% CO₂ for five or six days. CPE was monitored microscopically. The method of Reed and Muensch was used to calculate the cell culture-inhibiting dose 50% (CCID50).

In parallel, identical plates were set up that were analyzed using the MTT assay (Promega). In this assay, living cells are quantified by colorimetric staining. Hereto, 20 μl MTT (7.5 mgr/ml in PBS) was added to the wells and incubated at 37° C., 5% CO₂ for two hours. The supernatant was removed and 100 μl of a 20:1 isopropanol/triton-X100 solution was added to the wells. The plates were put on a 96-well shaker for 3 to 5 minutes to solubilize the precipitated staining. Absorbance was measured at 540 nm and at 690 nm (background). By this assay, wells with proceeding CPE or full CPE can be distinguished.

Neutralization Assay

96-well plates with diluted human serum samples were thawed at 37° C., 5% CO₂. Adenovirus stocks diluted to 200 CCID50 per 50 μl were prepared and 50 μl aliquots were added to columns 1 to 11 of the plates with serum. Plates were incubated for one hour at 37° C., 5% CO₂. Then 50 μl PER.C6 cells at 6×10⁵/ml were dispensed in all wells and incubated for one day at 37° C., 5% CO₂. Supernatant was removed using fresh pipette tips for each row and 200 μl fresh medium was added to all wells to avoid toxic effects of the serum. Plates were incubated for another four days at 37° C., 5% CO₂. In addition, parallel control plates were set up in duplo with diluted positive control sera generated in rabbits and specific for each serotype to be tested in rows A and B and with negative control serum (FCS) in rows C and D. Also, in each of the rows E through H, a titration was performed as described above with steps of five times dilutions starting with 200 CCID50 of each virus to be tested. On day 5, one of the control plates was analyzed microscopically and with the MTT assay. The experimental titer was calculated from the control titration plate observed microscopically. If CPE was found to be complete, i.e., the first dilution in the control titration experiment analyzed by MTT shows clear cell death, all assay plates were processed. If not, the assay was allowed to proceed for one or more days until full CPE was apparent, after which all plates were processed. In most cases, the assay was terminated at day 5. For Ad1, 5, 33, 39, 42 and 43, the assay was left for six days and for Ad2 for eight days.

A serum sample is regarded as “non-neutralizing” when, at the highest serum concentration, a maximum protection of 40% is seen compared to controls without serum.

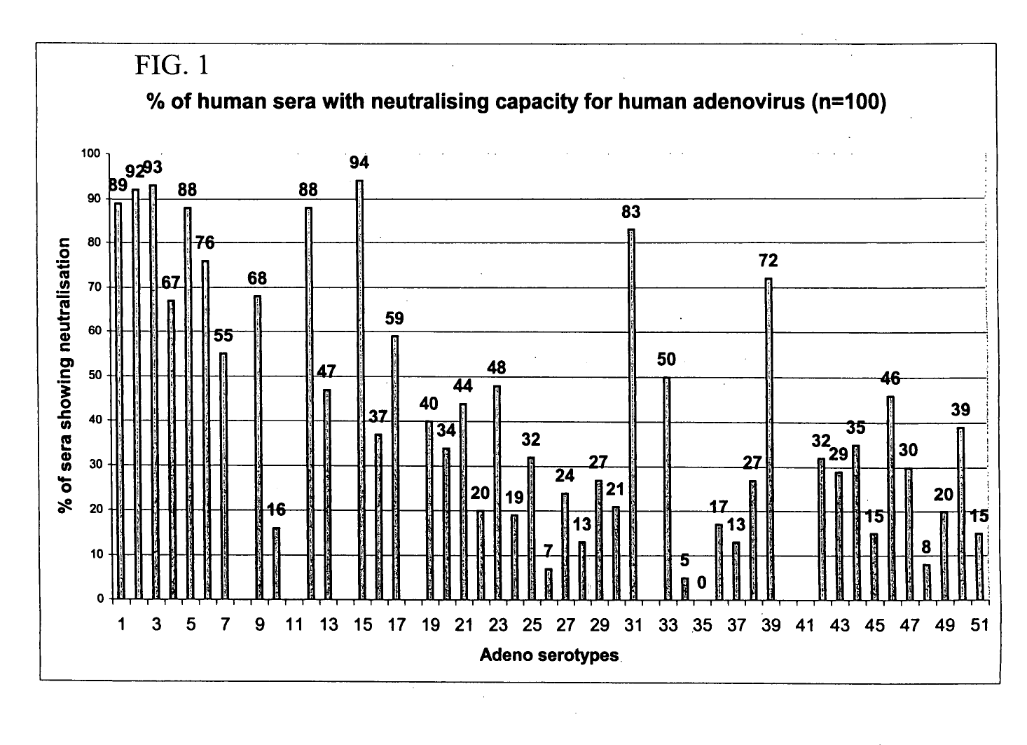

The results of the analysis of 44 prototype adenoviruses against serum from 100 healthy volunteers are shown in FIG. 1. As expected, the percentage of serum samples that contained neutralizing antibodies to Ad2 and Ad5 was very high. This was also true for most of the lower numbered adenoviruses. Surprisingly, none of the serum samples contained neutralizing antibodies to Ad35. Also, the number of individuals with neutralizing antibody titers to the serotypes 26, 34 and 48 was very low. Therefore, recombinant E1-deleted adenoviruses based on Ad35 or one of the other above-mentioned serotypes have an important advantage compared to recombinant vectors based on Ad5 with respect to clearance of the viruses by neutralizing antibodies.

Also, Ad5-based vectors that have (parts of) the capsid proteins involved in immunogenic response of the host replaced by the corresponding (parts of) capsid proteins of Ad35 or one of the other serotypes will be less, or even not, neutralized by the vast majority of human sera.

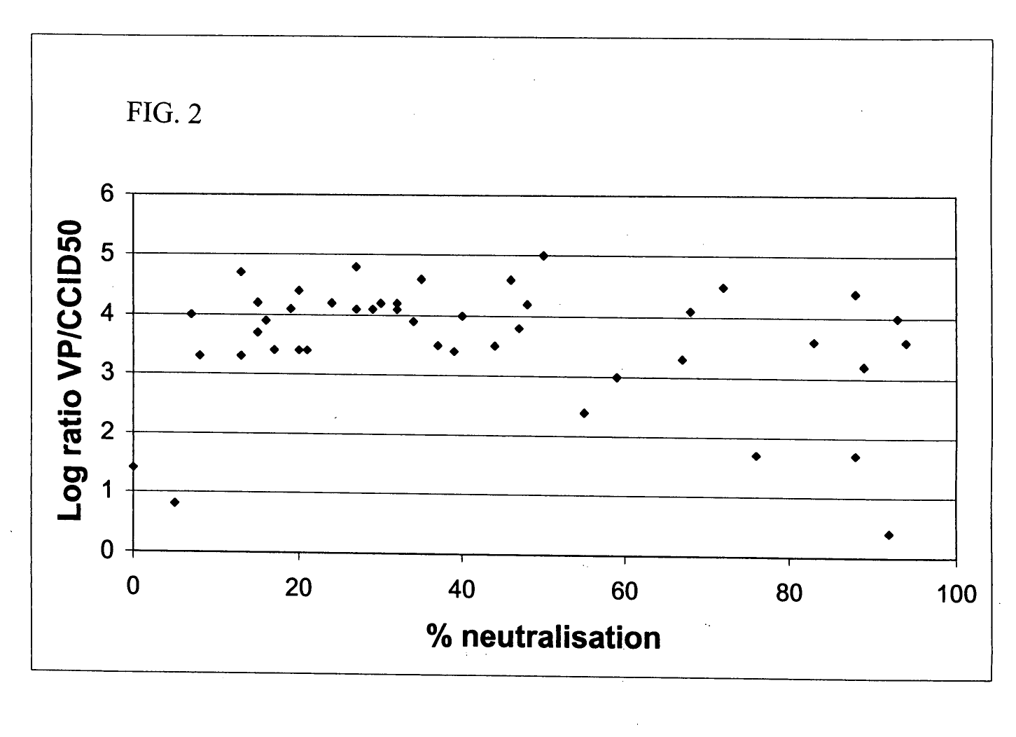

As can be seen in Table I, the VP/CCID50 ratio calculated from the virus particles per ml and the CCID50 obtained for each virus in the experiments was highly variable and ranged from 0.4 to 5 log. This is probably caused by different infection efficiencies of PER.C6 cells and by differences in replication efficiency of the viruses. Furthermore, differences in batch qualities may play a role. A high VP/CCID50 ratio means that more viruses were put in the wells to obtain CPE in five days. As a consequence, the outcome of the neutralization study might be biased since more (inactive) virus particles could shield the antibodies. To check whether this phenomenon had taken place, the VP/CCID50 ratio was plotted against the percentage of serum samples found positive in the assay (FIG. 2). The graph clearly shows that there is no negative correlation between the amount of viruses in the assay and neutralization in serum.

### Example 2

Generation of Ad5 Plasmid Vectors for the Production of Recombinant Viruses and Easy Manipulation of Adenoviral Genes

pBr/Ad.Bam-rITR (ECACC Deposit P97082122)

In order to facilitate blunt-end cloning of the ITR sequences, wild-type human adenovirus type 5 (Ad5) DNA was treated with Klenow enzyme in the presence of excess dNTPs. After inactivation of the Klenow enzyme and purification by phenol/chloroform extraction followed by ethanol precipitation, the DNA was digested with BamHI. This DNA preparation was used without further purification in a ligation reaction with pBr322-derived vector DNA prepared as follows: pBr322 DNA was digested with EcoRV and BamHI, dephosphorylated by treatment with TSAP enzyme (Life Technologies) and purified on LMP agarose gel (SeaPlaque GTG). After transformation into competent E. coli DH5α (Life Techn.) and analysis of ampicillin-resistant colonies, one clone was selected that showed a digestion pattern as expected for an insert extending from the BamHI site in Ad5 to the right ITR.

Sequence analysis of the cloning border at the right ITR revealed that the most 3′G residue of the ITR was missing, the remainder of the ITR was found to be correct. The missing G residue is complemented by the other ITR during replication.

pBr/Ad. Sal-rITR (ECACC Deposit P97082119)

pBr/Ad.Bam-rITR was digested with BamHI and SalI. The vector fragment including the adenovirus insert was isolated in LMP agarose (SeaPlaque GTG) and ligated to a 4.8 kb SalI-BamHI fragment obtained from wt Ad5 DNA and purified with the GENECLEAN II kit (Bio 101, Inc.). One clone was chosen and the integrity of the Ad5 sequences was determined by restriction enzyme analysis. Clone pBr/Ad.Sal-rITR contains Ad5 sequences from the SalI site at bp 16746 up to and including the rITR (missing the most 3′ G residue).

pBr/Ad.Cla-Bam (ECACC Deposit P97082117)

Wild-type (“wt”) Ad5 DNA was digested with ClaI and BamHI, and the 20.6 kb fragment was isolated from gel by electro-elution. pBr322 was digested with the same enzymes and purified from agarose gel by GENECLEAN. Both fragments were ligated and transformed into competent DH5α. The resulting clone pBr/Ad.Cla-Bam was analyzed by restriction enzyme digestion and shown to contain an insert with adenovirus sequences from bp 919 to 21566.

pBr/Ad.AflII-Bam (ECACC Deposit P97082114)

Clone pBr/Ad.Cla-Bam was linearized with EcoRI (in pBr322) and partially digested with AflII. After heat inactivation of AflII for 20 minutes at 65° C., the fragment ends were filled in with Klenow enzyme. The DNA was then ligated to a blunt double-stranded oligo linker containing a PacI site (5′-AATTGTCTTAATTAACCGCTTAA-3′ (SEQ ID NO:1)). This linker was made by annealing the following two oligonucleotides: 5′-AATTGTCTTAATTAACCGC-3′ (SEQ ID NO:2) and 5′-AATTGCGGTTAATTAAGAC-3′ (SEQ ID NO:3), followed by blunting with Klenow enzyme. After precipitation of the ligated DNA to change buffer, the ligations were digested with an excess PacI enzyme to remove concatameres of the oligo. The 22016 bp partial fragment containing Ad5 sequences from bp 3534 up to 21566 and the vector sequences, was isolated in LMP agarose (SeaPlaque GTG), re-ligated and transformed into competent DH5α. One clone that was found to contain the PacI site and that had retained the large adeno fragment was selected and sequenced at the 5′ end to verify correct insertion of the PacI linker in the (lost) AfII site.

pBr/Ad.Bam-rITRpac#2 (ECACC Deposit P97082120) and pBr/Ad.Bam-rITRpac#8 (ECACC Deposit P97082121)

To allow insertion of a PacI site near the ITR of Ad5 in clone pBr/Ad.Bam-rITR, about 190 nucleotides were removed between the ClaI site in the pBr322 backbone and the start of the ITR sequences. This was done as follows: pBr/Ad.Bam-rITR was digested with ClaI and treated with nuclease Bal31 for varying lengths of time (2, 5, 10 and 15 minutes). The extent of nucleotide removal was followed by separate reactions on pBr322 DNA (also digested at the ClaI site), using identical buffers and conditions. Bal31 enzyme was inactivated by incubation at 75° C. for 10 minutes, the DNA was precipitated and re-suspended in a smaller volume TE buffer. To ensure blunt ends, DNAs were further treated with T4 DNA polymerase in the presence of excess dNTPs. After digestion of the (control) pBr322 DNA with SalI, satisfactory degradation (˜150 bp) was observed in the samples treated for 10 or 15 minutes. The 10- or 15-minute treated pBr/Ad.Bam-rITR samples were then ligated to the above described blunted PacI linkers (See pBr/Ad.AflII-Bam). Ligations were purified by precipitation, digested with excess PacI and separated from the linkers on an LMP agarose gel. After re-ligation, DNAs were transformed into competent DH5α and colonies analyzed. Ten clones were selected that showed a deletion of approximately the desired length and these were further analyzed by T-track sequencing (T7 sequencing kit, Pharmacia Biotech). Two clones were found with the PacI linker inserted just downstream of the rITR. After digestion with PacI, clone #2 has 28 bp and clone #8 has 27 bp attached to the ITR.

pWE/Ad.AflII-rITR (ECACC Deposit P97082116)

Cosmid vector pWE15 (Clontech) was used to clone larger Ad5 inserts. First, a linker containing a unique PacI site was inserted in the EcoRI sites of pWE 15 creating pWE.pac. To this end, the double-stranded PacI oligo as described for pBr/Ad.AflII-BamHI was used but now with its EcoRI protruding ends. The following fragments were then isolated by electro-elution from agarose gel: pWE.pac digested with PacI, pBr/AflII-Bam digested with PacI and BamHI and pBr/Ad.Bam-rITR#2 digested with BamHI and PacI. These fragments were ligated together and packaged using λ phage packaging extracts (Stratagene) according to the manufacturer's protocol. After infection into host bacteria, colonies were grown on plates and analyzed for presence of the complete insert. pWE/Ad.AflII-rITR contains all adenovirus type 5 sequences from bp 3534 (AflII site) up to and including the right ITR (missing the most 3′ G residue).

pBr/Ad.lITR-Sal(9.4) (ECACC Deposit P97082115)

Adeno 5 wt DNA was treated with Klenow enzyme in the presence of excess dNTPs and subsequently digested with SalI. Two of the resulting fragments, designated left ITR-Sal(9.4) and Sal(16.7)-right ITR, respectively, were isolated in LMP agarose (Seaplaque GTG). pBr322 DNA was digested with EcoRV and SalI and treated with phosphatase (Life Technologies). The vector fragment was isolated using the GENECLEAN method (BIO 101, Inc.) and ligated to the Ad5 SalI fragments. Only the ligation with the 9.4 kb fragment gave colonies with an insert. After analysis and sequencing of the cloning border, a clone was chosen that contained the full ITR sequence and extended to the SalI site at bp 9462.

pBr/Ad.lITR-Sal(16.7) (ECACC Deposit P97082118)

pBr/Ad.lITR-Sal(9.4) is digested with SalI and dephosphorylated (TSAP, Life Technologies). To extend this clone up to the third SalI site in Ad5, pBr/Ad.Cla-Bam was linearized with BamHI and partially digested with SalI. A 7.3 kb SalI fragment containing adenovirus sequences from 9462-16746 was isolated in LMP agarose gel and ligated to the SalI-digested pBr/Ad.lITR-Sal(9.4) vector fragment.

pWE/Ad.AflII-EcoRI

pWE.pac was digested with ClaI and 5′ protruding ends were filled using Klenow enzyme. The DNA was then digested with PacI and isolated from agarose gel. pWE/AflII-rITR was digested with EcoRI and after treatment with Klenow enzyme digested with PacI. The large 24 kb fragment containing the adenoviral sequences was isolated from agarose gel and ligated to the ClaI-digested and blunted pWE.pac vector using the Ligation Express™ kit from Clontech. After transformation of Ultracompetent XL10-Gold cells from Stratagene, clones were identified that contained the expected insert. pWE/AflII-EcoRI contains Ad5 sequences from bp 3534-27336.

Generation of pWE/Ad.AflII-rITRsp

The 3′ ITR in the vector pWE/Ad.AflII-rITR does not include the terminal G-nucleotide. Furthermore, the PacI site is located almost 30 bp from the right ITR. Both these characteristics may decrease the efficiency of virus generation due to inefficient initiation of replication at the 3′ ITR. Note that during virus generation, the left ITR in the adapter plasmid is intact and enables replication of the virus DNA after homologous recombination.

To improve the efficiency of initiation of replication at the 3′ ITR, the pWE/Ad.AflII-rITR was modified as follows: construct pBr/Ad.Bam-rITRpac#2 was first digested with PacI and then partially digested with AvrII and the 17.8 kb vector-containing fragment was isolated and dephosphorylated using SAP enzyme (Boehringer Mannheim). This fragment lacks the adenovirus sequences from nucleotide 35464 to the 3′ ITR. Using DNA from pWE/Ad.AflII-rITR as template and the primers ITR-EPH: 5′-CGG AAT TCT TAA TTA AGT TAA CAT CAT CAA TAA TAT ACC-3′ (SEQ ID NO:4) and Ad101: 5′-TGA TTC ACA TCG GTC AGT GC-3′ (SEQ ID NO:5)

A 630 bp PCR fragment was generated corresponding to the 3′ Ad5 sequences. This PCR fragment was subsequently cloned in the vector pCR2.1 (Invitrogen) and clones containing the PCR fragment were isolated and sequenced to check correct DNA amplification. The PCR clone was then digested with PacI and AvrII and the 0.5 kb adeno insert was ligated to the PacI/partial AvrII-digested pBr/Ad.Bam-rITRpac#2 fragment generating pBr/Ad.Bam-rITRsp. Next, this construct was used to generate a cosmid clone (as previously described herein) that has an insert corresponding to the adenovirus sequences 3534 to 35938. This clone was designated pWE/AflII-rITRsp.

Generation of pWE/Ad.AflII-rITRΔE2A

Deletion of the E2A-coding sequences from pWE/Ad.AflII-rITR (ECACC deposit P97082116) has been accomplished as follows. The adenoviral sequences flanking the E2A-coding region at the left and the right site were amplified from the plasmid pBr/Ad.Sal.rITR (ECACC deposit P97082119) in a PCR reaction with the Expand PCR system (Boehringer) according to the manufacturer's protocol. The following primers were used:

Right-flanking sequences (corresponding Ad5 nucleotides 24033 to 25180):

ΔE2A.SnaBI:

(SEQ ID NO:6)

5′-GGC GTA CGT AGC CCT GTC GAA AG-3′
ΔE2A.DBP-start:

(SEQ ID NO:7)

5′-CCA ATG CATTCG AAG TAC TTC
CTT CTC CTA TAG GC-3′.

The amplified DNA fragment was digested with SnaBI and NsiI (NsiI site is generated in the primer ΔE2A.DBP-start, underlined).

Left-flanking sequences (corresponding Ad5 nucleotides 21557 to 22442):

| | |
|---|---|
| ΔE2A.DBP-stop: | |
| 5′-CCA ATG CATACG GCG CAG ACG G-3′ | (SEQ ID NO:8) |
| ΔE2A.BamHI: | |
| 5′-GAG GTG GAT CCC ATG GAC GAG-3′. | (SEQ ID NO:9) |

The amplified DNA was digested with BamHI and NsiI (NsiI site is generated in the primer ΔE2A.DBP-stop, underlined). Subsequently, the digested DNA fragments were ligated into SnaBI/BamHI digested pBr/Ad.Sal-rITR. Sequencing confirmed the exact replacement of the DBP-coding region with a unique NsiI site in plasmid pBr/Ad.Sal-rITRΔE2A. The unique NsiI site can be used to introduce an expression cassette for a gene to be transduced by the recombinant vector.

The deletion of the E2A-coding sequences was performed such that the splice acceptor sites of the 100K encoding L4-gene at position 24048 in the top strand was left intact. In addition, the poly adenylation signals of the original E2A-RNA and L3-RNAs at the left-hand site of the E2A-coding sequences were left intact. This ensures proper expression of the L3-genes and the gene encoding the 100K L4-protein during the adenovirus life cycle.

Next, the plasmid pWE/Ad.AflII-rITRΔE2A was generated. The plasmid pBr/Ad.Sal-rITRΔE2A was digested with BamHI and SpeI. The 3.9-Kb fragment in which the unique NsiI site replaced the E2A-coding region was isolated. The pWE/Ad.AflII-rITR was digested with BamHI and SpeI. The 35 Kb DNA fragment, from which the BamHI/SpeI fragment containing the E2A-coding sequence was removed, was isolated. The fragments were ligated and packaged using λ phage-packaging extracts according to the manufacturer protocol (Stratagene), yielding the plasmid pWE/Ad.AflII-rITRΔE2A.

This cosmid clone can be used to generate adenoviral vectors that are deleted for E2A by co-transfection of PacI-digested DNA together with digested adapter plasmids onto packaging cells that express functional E2A gene product.

Construction of Adapter Plasmids

The absence of sequence overlap between the recombinant adenovirus and E1 sequences in the packaging cell line is essential for safe, RCA-free generation and propagation of new recombinant viruses. The adapter plasmid pMLPI.TK (described in U.S. Pat. No. 5,994,128 to Bout et al.) is an example of an adapter plasmid designed for use according to the invention in combination with the improved packaging cell lines of the invention. This plasmid was used as the starting material to make a new vector in which nucleic acid molecules comprising specific promoter and gene sequences can be easily exchanged.

First, a PCR fragment was generated from pZipΔMo+PyF101(N⁻) template DNA (described in PCT/NL96/00195) with the following primers: LTR-1: 5′-CTG TAC GTA CCA GTG CAC TGG CCT AGG CAT GGA AAA ATA CAT AAC TG-3′ (SEQ ID NO:10) and LTR-2: 5′-GCG GAT CCT TCG AAC CAT GGT AAG CTT GGT ACC GCT AGC GTT AAC CGG GCG ACT CAG TCA ATC G-3′ (SEQ ID NO: 11). Pwo DNA polymerase (Boehringer Mannheim) was used according to manufacturer's protocol with the following temperature cycles: once for 5 minutes at 95° C.; 3 minutes at 55° C.; and 1 minute at 72° C., and 30 cycles of 1 minute at 95° C., 1 minute at 60° C., 1 minute at 72° C., followed by once for 10 minutes at 72° C. The PCR product was then digested with BamHI and ligated into pMLP10 (Levrero et al., 1991) vector digested with PvuII and BamHI, thereby generating vector pLTR10. This vector contains adenoviral sequences from bp 1 up to bp 454 followed by a promoter consisting of a part of the Mo-MuLV LTR having its wild-type enhancer sequences replaced by the enhancer from a mutant polyoma virus (PyF101). The promoter fragment was designated L420. Next, the coding region of the murine HSA gene was inserted. pLTR10 was digested with BstBI followed by Klenow treatment and digestion with NcoI. The HSA gene was obtained by PCR amplification on pUC18-HSA (Kay et al., 1990) using the following primers: HSA1, 5′-GCG CCA CCA TGG GCA GAG CGA TGG TGG C-3′ (SEQ ID NO:12) and HSA2, 5′-GTT AGA TCT AAG CTT GTC GAC ATC GAT CTA CTA ACA GTA GAG ATG TAG AA-3′ (SEQ ID NO:13). The 269 bp amplified fragment was subcloned in a shuttle vector using the NcoI and BgllI sites. Sequencing confirmed incorporation of the correct coding sequence of the HSA gene, but with an extra TAG insertion directly following the TAG stop codon. The coding region of the HSA gene, including the TAG duplication was then excised as a NcoI (sticky)-SalI (blunt) fragment and cloned into the 3.5 kb NcoI (sticky)/BstBI (blunt) fragment from pLTR10, resulting in pLTR-HSA10.

Finally, pLTR-HSA10 was digested with EcoRI and BamHI after which the fragment containing the left ITR, packaging signal, L420 promoter and HSA gene was inserted into vector pMLPI.TK digested with the same enzymes and thereby replacing the promoter and gene sequences. This resulted in the new adapter plasmid pAd/L420-HSA that contains convenient recognition sites for various restriction enzymes around the promoter and gene sequences. SnaBI and AvrII can be combined with HpaI, NheI, KpnI, HindIII to exchange promoter sequences, while the latter sites can be combined with the ClaI or BamHI sites 3′ from HSA-coding region to replace genes in this construct.

Replacing the promoter, gene and poly A sequences in pAd/L420-HSA with the CMV promoter, a multiple cloning site, an intron and a poly-A signal made another adapter plasmid that was designed to allow easy exchange of nucleic acid molecules. For this purpose, pAd/L420-HSA was digested with AvrII and BglII followed by treatment with Klenow to obtain blunt ends. The 5.1 kb fragment with pBr322 vector and adenoviral sequences was isolated and ligated to a blunt 1570 bp fragment from pcDNA1/amp (Invitrogen) obtained by digestion with HhaI and AvrII followed by treatment with T4 DNA polymerase. This adapter plasmid was designated pAd5/CLIP.

To enable removal of vector sequences from the left ITR in pAd5/Clip, this plasmid was partially digested with EcoRI and the linear fragment was isolated. An oligo of the sequence 5′ TTAAGTCGAC-3′ (SEQ ID NO:14) was annealed to itself resulting in a linker with a SalI site and EcoRI overhang. The linker was ligated to the partially digested pAd5/Clip vector and clones were selected that had the linker inserted in the EcoRI site 23 bp upstream of the left adenovirus ITR in pAd5/Clip resulting in pAd5/Clipsal. Likewise, the EcoRI site in pAd5/Clip has been changed to a PacI site by insertion of a linker of the sequence 5′-AATTGTCTTAATTAACCGCAATT-3′ (SEQ ID NO:15). The pAd5/Clip vector was partially digested with EcoRI, dephosphorylated and ligated to the PacI linker with EcoRI overhang. The ligation mixture was digested with PacI to remove concatamers, isolated from agarose gel and religated. The resulting vector was designated pAd5/Clippac. These changes enable more flexibility to liberate the left ITR from the plasmid vector sequences.

The vector pAd5/L420-HSA was also modified to create a SalI or PacI site upstream of the left ITR. Hereto, pAd5/L420-HSA was digested with EcoRI and ligated to the previously herein described PacI linker. The ligation mixture was digested with PacI and religated after isolation of the linear DNA from agarose gel to remove concatamerized linkers. This resulted in adapter plasmid pAd5/L420-HSApac. This construct was used to generate pAd5/L420-HSAsal as follows: pAd5/L420-HSApac was digested with ScaI and BsrGI and the vector fragment was ligated to the 0.3 kb fragment isolated after digestion of pAd5/Clipsal with the same enzymes.

Generation of Adapter Plasmids pAdMire and pAdApt

To create an adapter plasmid that only contains a polylinker sequence and no promoter or polyA sequences, pAd5/L420-HSApac was digested with AvrII and BglII. The vector fragment was ligated to a linker oligonucleotide digested with the same restriction enzymes. Annealing oligos of the following sequence made the linker:

PLL-1:

(SEQ ID NO:16)

5′-GCC ATC CCT AGG AAG CTT GGT ACC GGT GAA TTC
GCT AGC GTT AAC GGA TCC TCT AGA CGA GAT CTG G-3′
and
PLL-2:

(SEQ ID NO:17)

5′-CCA GAT CTC GTC TAG AGG ATC CGT TAA CGC TAG
CGA ATT CAC CGG TAC CAA GCT TCC TAG GGA TGG C-3′.

The annealed linkers were digested with AvrII and BglII and separated from small ends by column purification (Qiaquick nucleotide removal kit) according to manufacturer's recommendations. The linker was then ligated to the AvrlI/BglI-digested pAd5/L420-HSApac fragment. A clone, designated AdMire, was selected that had the linker incorporated and was sequenced to check the integrity of the insert.

Adapter plasmid AdMire enables easy insertion of complete expression cassettes. An adapter plasmid containing the human CMV promoter that mediates high expression levels in human cells was constructed as follows: pAd5/L420-HSApac was digested with AvrII and 5′ protruding ends were filled in using Klenow enzyme. A second digestion with HindIII resulted in removal of the L420 promoter sequences. The vector fragment was isolated and ligated to a PCR fragment containing the CMV promoter sequence. This PCR fragment was obtained after amplification of CMV sequences from pCMVLacI (Stratagene) with the following primers:

CMV plus: 5′-GAT CGG TAC CAC TGC AGT GGT CAA TAT TGG CCA TTA GCC-3′ (SEQ ID NO:18) and CMVminA: 5′-GAT CAA GCT TCC AAT GCA CCG TTC CCG GC-3′ (SEQ ID NO:19). The PCR fragment was first digested with PstI (underlined in CMV plus), after which the 3′-protruding ends were removed by treatment with T4 DNA polymerase. Then the DNA was digested with HindIII (underlined in CMVminA) and ligated into the herein-described pAd5/L420-HSApac vector fragment digested with AvrII and HindIII. The resulting plasmid was designated pAd5/CMV-HSApac. This plasmid was then digested with HindIII and BamHI and the vector fragment was isolated and ligated to the polylinker sequence obtained after digestion of AdMire with HindIII and BglII. The resulting plasmid was designated pAdApt. Adapter plasmid pAdApt contains nucleotides −735 to +95 of the human CMV promoter (Boshart et al., 1985). A second version of this adapter plasmid containing a SalI site in place of the PacI site upstream of the left ITR was made by inserting the 0.7 kb ScaI-BsrGI fragment from pAd5/Clipsal into pAdApt digested with ScaI and partially digested with BsrGI. This clone was designated pAdApt.sal.

Generation of Recombinant Adenoviruses Based on Ad5

RCA-free recombinant adenoviruses can be generated very efficiently using the herein-described adapter plasmids and the pWe/Ad.AflII-rITR or pWE/Ad.AflII-rITrsp constructs. Generally, the adapter plasmid containing the desired transgene in the desired expression cassette is digested with suitable enzymes to liberate the insert from vector sequences at the 3′ and/or at the 5′ end. The adenoviral complementation plasmids pWE/Ad.AflII-rITR or pWE/Ad.AflII-rITRsp are digested with PacI to liberate the adeno sequences from the vector plasmids. As a non-limiting example, the generation of AdApt-LacZ is described. Adapter plasmid pAdApt-LacZ was generated as follows. The E. coli LacZ gene was amplified from the plasmid pMLP.nlsLacZ (EP 95-202 213) by PCR with the primers 5′-GGG GTG GCC AGG GTA CCT CTA GGC TTT TGC AA-3′ (SEQ ID NO:20) and 5′-GGG GGG ATC CAT AAA CAA GTT CAG AAT CC-3′ (SEQ ID NO:21). The PCR reaction was performed with Ex Taq (Takara) according to the suppliers protocol at the following amplification program: 5 minutes at 94° C., 1 cycle; 45 seconds at 94° C. and 30 seconds at 60° C. and 2 minutes at 72° C., 5 cycles; 45 seconds at 94° C. and 30 seconds at 65° C. and 2 minutes at 72° C., 25 cycles; 10 minutes at 72° C.; 45 seconds at 94° C. and 30 seconds at 60° C. and 2 minutes at 72° C., 5 cycles, I cycle. The PCR product was subsequently digested with KpnI and BamHI and the digested DNA fragment was ligated into KpnI/BamHI digested pcDNA3 (Invitrogen), giving rise to pcDNA3.nlsLacZ. Construct pcDNA3.nlsLacZ was then digested with KpnI and BamHI and the 3 kb LacZ fragment was isolated from gel using the GENECLEAN spin kit (Bio 101, Inc.). pAdApt was also digested with KpnI and BamHI and the linear vector fragment was isolated from gel as above. Both isolated fragments were ligated and one clone containing the LacZ insert was selected. Construct pAdApt-LacZ was digested with SalI, purified by the GENECLEAN spin kit and subsequently digested with PacI. pWE/Ad.AflII-rITRsp was digested with PacI. Both digestion mixtures were treated for 30 minutes at 65° C. to inactivate the enzymes. Samples were put on gel to estimate the concentration. 2.5×10⁶ PER.C6 cells were seeded in T25 flasks in DMEM with 10% FCS and 10 mM MgCl. The next day, four micrograms of each plasmid was transfected into PER.C6 cells using lipofectamine transfection reagents (Life Technologies Inc.) according to instructions of the manufacturer. The next day, the medium was replaced by fresh culture medium and cells were further cultured at 37° C., 10% CO₂. Again, one day later, cells were trypsinised, seeded into T80 flasks and cultured at 37° C., 10% CO₂. Full CPE was obtained six days after seeding in the T80 flask. Cells were harvested in the medium and subjected to one freeze/thaw cycle. The crude lysate obtained this way was used to plaque purify the mixture of viruses. Ten plaques were picked, expanded in a 24-well plate and tested for LacZ expression following infection of A549 cells. Viruses from all ten plaques expressed LacZ.

### Example 3

Generation of Chimeric-Recombinant Adenoviruses

Generation of Hexon Chimeric Ad5-Based Adenoviruses

Neutralizing antibodies in human serum are mainly directed to the hexon protein and, to a lesser extent, to the penton protein. Hexon proteins from different serotypes show highly variable regions present in loops that are predicted to be exposed at the outside of the virus (Athappilly et al., 1994, J. Mol. Biol. 242, 430-455). Most type-specific epitopes have been mapped to these highly variable regions (Toogood et al., 1989, J. Gen Virol. 70, 3203-3214). Thus, replacement of (part of) the hexon sequences with corresponding sequences from a different serotype is an effective strategy to circumvent (pre-existing) neutralizing antibodies to Ad5. Hexon-coding sequences of Ad5 are located between nucleotides 18841 and 21697.

To facilitate easy exchange of hexon-coding sequences from alternative adenovirus serotypes into the Ad5 backbone, a shuttle vector was generated first. This sub-clone, coded pBr/Ad.Eco-PmeI, was generated by first digesting plasmid pBr322 with EcoRI and EcoRV and inserting the 14 kb PmeI-EcoRI fragment from pWE/Ad.AflII-Eco. In this shuttle vector, a deletion was made of a 1430 bp SanDI fragment by digestion with SanDI and re-ligation to give pBr/Ad.Eco-PmeIΔSanDI. The removed fragment contains unique SpeI and MunI sites. From pBr/Ad.Eco-PmeIΔSanDI, the Ad5 DNA-encoding hexon was deleted. Hereto, the hexon flanking sequences were PCR amplified and linked together, thereby generating unique restriction sites replacing the hexon-coding region. For these PCR reactions, four different oligonucleotides were required: Δhex1-Δhex4.

Δhex1:

(SEQ ID NO:22)

5′-CCT GGT GCT GCC AAC AGC-3′
Δhex2:

(SEQ ID NO:23)

5′-CCG GAT CCA CTA GTG GAA AGC GGG CGC GCG-3′
Δhex3:

(SEQ ID NO:24)

5′-CCG GAT CCA ATT GAG AAG CAA GCA ACA TCA
ACA AC-3′
Δhex4:

(SEQ ID NO:25)

5′-GAG AAG GGC ATG GAG GCT G-3′

The amplified DNA product of ±1100 bp obtained with oligonucleotides Δhex1 and Δhex2 was digested with BamHI and FseI. The amplified DNA product of ±1600 bp obtained with oligonucleotides Δhex3 and Δhex4 was digested with BamHI and SbfI. These digested PCR fragments were subsequently purified from agarose gel and in a tri-part ligation reaction using T4 ligase enzyme linked to pBr/Ad.Eco-PmeI ΔSanDI digested with FseI and SbfI. The resulting construct was coded pBr/Ad.Eco-PmeΔHexon. This construct was sequenced in part to confirm the correct nucleotide sequence and the presence of unique restriction sites MunI and SpeI.

pBr/Ad.Eco-PmeΔHexon serves as a shuttle vector to introduce heterologous hexon sequences amplified from virus DNA from different serotypes using primers that introduce the unique restriction sites MunI and SpeI at the 5′ and 3′ ends of the hexon sequences respectively. To generate Ad5-based vectors that contain hexon sequences from the serotypes to which healthy individuals have no, or very low, titers of NAB, the hexon sequences of Ad35, Ad34, Ad26 and Ad48 were amplified using the following primers:
Hex-up2: 5′-GAC TAG TCA AGA TGG CYA CCC CHT CGA TGA TG-3′ (SEQ ID NO:26) (where Y can be a C or T and H can be an A, T or C as both are degenerate oligo nucleotides); and
Hex-do2: 5′-GCT GGC CAA TTG TTA TGT KGT KGC GTT RCC GGC-3′ (SEQ ID NO:27) (where K can be a T or G and R can be an A or G as both are degenerate oligo nucleotides).

These primers were designed using the sequences of published hexon-coding regions (for example, hexon sequences of Ad2, Ad3, Ad4, Ad5, Ad7, Ad16, Ad40 and Ad41 can be obtained at Genbank). Degenerated nucleotides were incorporated at positions that show variation between serotypes.

PCR products were digested with SpeI and MunI and cloned into the pBr/Ad.Eco-PmeΔHexon construct digested with the same enzymes.

The hexon-modified sequences were subsequently introduced in the construct pWE/Ad.AflII-rITR by exchange of the AscI fragment generating pWE/Ad.AflII-rITRHexXX where XX stands for the serotype used to amplify hexon sequences.

The pWE/Ad.AflII-rITRHexXX constructs are then used to make viruses in the same manner as previously described herein for Ad5-recombinant viruses.

Generation of Penton Chimeric Ad5-Based Recombinant Viruses

The adenovirus type 5 penton gene is located between sequences 14156 and 15869. Penton base is the adenovirus capsid protein that mediates internalization of the virus into the target cell. At least some serotypes (type C and B) have been shown to achieve this by interaction of an RGD sequence in penton with integrins on the cell surface. However, type F adenoviruses do not have an RGD sequence and for most viruses of the A and D group, the penton sequence is not known. Therefore, the penton may be involved in target cell specificity. Furthermore, as a capsid protein, the penton protein is involved in the immunogenicity of the adenovirus (Gahery-Segard et al., 1998). Therefore, replacement of Ad5 penton sequences with penton sequences from serotypes to which no or low titers of NAB exist, in addition to replacement of the hexon sequences, will prevent clearance of the adenoviral vector more efficiently than replacement of hexon alone. Replacement of penton sequences may also affect infection specificity.

To be able to introduce heterologous penton sequences in Ad5, we made use of the plasmid-based system described above. First, a shuttle vector for penton sequences was made by insertion of the 7.2 kb NheI-EcoRV fragment from construct pWE/Ad.AflII-EcoRI into pBr322 digested with the same enzymes. The resulting vector was designated pBr/XN. From this plasmid, Ad5 penton sequences were deleted and replaced by unique restriction sites that are then used to introduce new penton sequences from other serotypes. Hereto, the left-flanking sequences of penton in pBr/XN were PCR amplified using the following primers: DP5-F: 5′-CTG TTG CTG CTG CTA ATA GC-3′ (SEQ ID NO:28) and DP5-R: 5′-CGC GGA TCC TGT ACA ACT AAG GGG AAT ACA AG-3′ (SEQ ID NO:29).

DP5-R has a BamHI site (underlined) for ligation to the right-flanking sequence and also introduces a unique BsrGI site (bold face) at the 5′-end of the former Ad5 penton region. The right-flanking sequence was amplified using: DP3-F: 5′-CGC GGA TCC CTT AAG GCA AGC ATG TCC ATC CTT-3′ (SEQ ID NO:30) and DP3-3R: 5′-AAA ACA CGT TTT ACG CGT CGA CCT TTC-3′ (SEQ ID NO:31). DP3-F has a BamHI site (underlined) for ligation to the left-flanking sequence and also introduces a unique AflI site (bold face) at the 3′-end of the former Ad5 penton region. The two resulting PCR fragments were digested with BamHI and ligated together. Then, this ligation mixture was digested with AvrII and BglII. pBr/XN was also digested with AvrII and BglII and the vector fragment was ligated to the digested ligated PCR fragments. The resulting clone was designated pBr/Ad.Δpenton. Penton-coding sequences from Ad35, Ad34, Ad26 and Ad48 were PCR amplified, such that the 5′ and 3′ ends contained the BsrGI and AflII sites, respectively. Hereto, the following primers were used:

For Ad34 and Ad35:
P3-for:

(SEQ ID NO:32)

5′-GCT CGA TGT ACA ATG AGG AGA CGA GCC GTG CTA-3′
and
P3-rev:

(SEQ ID NO:33)

5′-GCT CGA CTT AAG TTA GAA AGT GCG GCT TGA AAG-3′.
t001621 For Ad26 and Ad4S:
P17F:

(SEQ ID NO:34)

5′-GCT CGA TGT ACA ATG AGG CGT GCG GTG GTG TCT TC-
3′
and
P17R:

(SEQ ID NO:35)

5′-GCT CGA CTT AAG TTA GAA GGT GCG ACT GGA AAG C-
3′.

Amplified PCR products were digested with BfrI and BsrGI and cloned into pBr/Ad.Δpenton digested with the same enzymes. Introduction of these heterologous penton sequences into the pBr/Ad.Δpenton generated constructs designated pBr/Ad.pentonXX wherein XX represents the number of the serotype corresponding to the serotype used to amplify the inserted penton sequences. Subsequently, the new penton sequences were introduced in the pWE/Ad.AflIII-rITR vector having a modified hexon. For example, penton sequences from Ad35 were introduced in the construct pWE/Ad.AfIII-rITRHex35 by exchange of the common FseI fragment. Other combinations of penton and hexon sequences were also made. Viruses with modified hexon and penton sequences were made as described above using co-transfection with an adapter plasmid on PER.C6 cells. In addition, penton sequences were introduced in the pWE/Ad.AfIII-rITR construct. The latter constructs contain only a modified penton and viruses generated from these constructs will be used to study the contribution of penton sequences to the neutralization of adenoviruses and also for analysis of possible changes in infection efficiency and specificity.

Generation of Fiber Chimeric Ad5-Based Viruses

Adenovirus infection is mediated by two capsid proteins, fiber and penton. Binding of the virus to the cells is achieved by interaction of the protruding fiber protein with a receptor on the cell surface. Internalization then takes place after interaction of the penton protein with integrins on the cell surface. At least some adenovirus from subgroups C and B have been shown to use a different receptor for cell binding and, therefore, have different infection efficiencies on different cell types. Thus, it is possible to change the infection spectrum of adenoviruses by changing the fiber in the capsid. The fiber-coding sequence of Ad5 is located between nucleotides 31042 and 32787. To remove the Ad5 DNA-encoding fiber, we started with construct pBr/Ad.Bam-rITR. First, an NdeI site was removed from this construct. For this purpose, pBr322 plasmid DNA was digested with NdeI. After which, protruding ends were filled using Klenow enzyme. This pBr322 plasmid was then re-ligated, digested with NdeI, and transformed into E. coli DH5α. The obtained pBr/ΔNdeI plasmid was digested with ScaI and SalI and the resulting 3198 bp vector fragment was ligated to the 15349 bp ScaI-SalI fragment derived from pBr/Ad.BamrITR, resulting in plasmid pBr/Ad.Bam-rITRΔNdeI, which hence contained a unique NdeI site. Next, a PCR was performed with oligonucleotides NY-up: 5′-CGA CAT ATG TAG ATG CAT TAG TTT GTG TTA TGT TTC AAC GTG-3′ (SEQ ID NO:36) and NY-down: 5′-GGA GAC CAC TGC CAT GTT-3′ (SEQ ID NO:37).

During amplification, both an NdeI (bold face) and an NsiI restriction site (underlined) were introduced to facilitate cloning of the amplified fiber DNAs. Amplification consisted of 25 cycles of each 45 seconds at 94° C., 1 minute at 60° C., and 45 seconds at 72° C. The PCR reaction contained 25 pmol of oligonucleotides NY-up or NY-down, 2 mM dNTP, PCR buffer with 1.5 mM MgCl₂, and 1 unit of Elongase heat stable polymerase (Gibco, The Netherlands). One-tenth of the PCR product was run on an agarose gel that demonstrated that the expected DNA fragment of ±2200 bp was amplified. This PCR fragment was subsequently purified using GENECLEAN kit system (Bio101 Inc.). Then, both the construct pBr/Ad.Bam-rITRΔNdeI, as well as the PCR product, were digested with restriction enzymes NdeI and SbfI. The PCR fragment was subsequently cloned using T4 ligase enzyme into the NdeI and SbfI digested pBr/Ad.Bam-rITRΔNdeI, generating pBr/Ad.BamRΔFib.

This plasmid allows insertion of any PCR-amplified fiber sequence through the unique NdeI and NsiI sites that are inserted in place of the removed fiber sequence. Viruses can be generated by a double-homologous recombination in packaging cells described in U.S. Pat. No. 5,994,128 to Bout et al. using an adapter plasmid, construct pBr/Ad.AflII-EcoRI digested with PacI and EcoRI and a pBr/Ad.BamRΔFib construct in which heterologous fiber sequences have been inserted. To increase the efficiency of virus generation, the construct pBr/Ad.BamRΔFib was modified to generate a PacI site flanking the right ITR. Hereto, pBr/Ad.BamRΔFib was digested with AvrII and the 5 kb adenovirus fragment was isolated and introduced into the vector pBr/Ad.Bam-rITR.pac#8 described above replacing the corresponding AvrII fragment. The resulting construct was designated pBr/Ad.BamRΔFib.pac.

Once a heterologous fiber sequence is introduced in pBr/Ad.BamRΔFib.pac, the fiber modified right-hand adenovirus clone is introduced into a large cosmid clone as previously described herein for pWE/Ad.AflII-rITR. Such a large cosmid clone allows generation of adenovirus by only one homologous recombination. Ad5-based viruses with modified fibers have been made and described (see, European Patent Appln. Nos. 98204482.8 and 99200624.7). In addition, hexon and penton sequences from serotypes from this invention are combined with the desired fiber sequences to generate viruses that infect the target cell of choice very efficiently. For example, smooth muscle cells, endothelial cells or synoviocytes (all from human origin) are very well infected with Ad5-based viruses with a fiber from subgroup B viruses especially Ad16.

The foregoing examples in which specific sequences can be deleted from the Ad5 backbone in the plasmids and replaced by corresponding sequences from other serotypes demonstrate the flexibility of the system. It is evident that by the methods described herein, any combination of capsid genes from different serotypes can be made. Thus, chimeric recombinant Ad5-based adenoviruses are designed with desired hexon and penton sequences making the virus less sensitive for neutralization and with desired fiber sequences allowing efficient infection in specific target tissues.

### Example 4

Construction of a Plasmid-Based System to Generate Ad35-Recombinant Viruses

Partial restriction maps of Ad35 have been published previously (Valderrama-Leon et al., 1985; Kang et al., 1989; Li et al., 1991). An example of a functional plasmid-based system to generate recombinant adenoviruses based on Ad35 consists of the following elements:

1. An adapter plasmid comprising a left ITR and packaging sequences derived from Ad35 and at least one restriction site for insertion of a heterologous expression cassette and lacking E1 sequences. Furthermore, the adapter plasmid contains Ad35 sequences 3′ from the E1B-coding region including the pIX promoter and coding sequences sufficient to mediate homologous recombination of the adapter plasmid with a second nucleotide.
2. A second nucleotide comprising sequences homologous to the adapter plasmid and Ad35 sequences necessary for the replication and packaging of the recombinant virus, that is, early, intermediate and late genes that are not present in the packaging cell.
3. A packaging cell providing at least functional E1 proteins and capable of complementing the E1 function of Ad35.

Ad35 DNA was isolated from a purified virus batch as follows. To 100 μl of virus stock (Ad35: 3.26×10¹² VP/ml), 10 μl 10× DNAse buffer (130 mM Tris-HCl pH 7.5; 1.2 M CaCl₂; 50 mM MgCl₂) was added. After addition of 10 μl, 10 mgr/ml DNAse I (Roche Diagnostics), the mixture was incubated for 1 hour at 37° C. Following addition of 2.5 μl 0.5 M EDTA, 3.2 μl 20% SDS and 1.5 μl ProteinaseK (Roche Diagnostics; 20 mgr/ml), samples were incubated at 50° C. for 1 hour. Next, the viral DNA was isolated using the GENECLEAN spin kit (Bio101 Inc.) according to the manufacturer's instructions. DNA was eluted from the spin column with 25 μl sterile MilliQ water.

In the following, sizes of DNA fragments and fragment numbering will be used according to Kang et al., 1989. Ad35 DNA was digested with EcoRI and the three fragments (approximately 22.3 (A), 7.3 (B) and 6 kb (C)) were isolated from gel using the GENECLEAN kit (Bio101, Inc.). pBr322 was digested with EcoRI or with EcoRI and EcoRV and digested fragments were isolated from gel and dephosphorylated with Tsap enzyme (Gibco BRL). Next, the 6 kb Ad35 C fragment was ligated to the pBr322xEcoRI fragment and the ITR-containing Ad35 fragment (EcoRI-B) was ligated to the pBr322xEcoRI/EcoRV fragment. Ligations were incubated at 16° C. overnight and transformed into DH5α-competent bacteria (Life Techn.). Minipreps of obtained colonies were analyzed for correct insertion of the Ad35 fragments by restriction analysis. Both the 6 kb and the 7.3 kb Ad35 fragments were found to be correctly inserted in pBr322. The 6 kb fragment was isolated in both orientations pBr/Ad35-Eco6.0⁺ and pBr/Ad35-Eco6.0⁻ whereby the + stands for 5′ to 3′ orientation relative to pBr322. The clone with the 7.3 kb Ad35 B insert, designated pBr/Ad35-Eco7.3 was partially sequenced to check correct ligation of the 3′ ITR. It was found that the ITR had at least the sequence 5′-CATCATCAAT . . . -3′ found in SEQ ID NO:40 in the lower strand. Then pBr/Ad35-Eco7.3 was extended to the 5′ end by insertion of the 6 kb Ad35 fragment. Hereto, pBr/Ad35-Eco7.3 was digested with EcoRI and dephosphorylated. The fragment was isolated from gel and ligated to the 6 kb Ad35 EcoRI fragment. After transformation, clones were tested for correct orientation of the insert and one clone was selected, designated pBr/Ad35-Eco13.3.

This clone is then extended with the ˜5.4 kb SalI D fragment obtained after digestion of wt Ad35 with SalI. Hereto, the SalI site in the pBr322 backbone is removed by partial digestion of pBr/Ad35-Eco13.3 with SalI, filling in of the sticky ends by Klenow treatment and re-ligation. One clone is selected that contains a single SalI site in the adenoviral insert. This clone, designated pBrΔsal/Ad35-Eco13.3 is then linearized with AatII which is present in the pBr322 backbone and ligated to a SalI linker with AatII complementary ends. The DNA is then digested with excess SalI and the linear fragment is isolated and ligated to the 5.4 kb SalI-D fragment from Ad35. One clone is selected that contains the SalI fragment inserted in the correct orientation in pBr/Ad35-Eco13.3. The resulting clone, pBr/Ad35.Sal2-rITR, contains the 3′ ˜17 kb of Ad35 including the right ITR. To enable liberation of the right ITR from the vector sequences at the time of virus generation, a NotI site flanking the right ITR is introduced by PCR.

The Ad35 EcoRI-A fragment of 22.3 kb was also cloned in pBr322xEcoRI/EcoRV. One clone, designated pBr/Ad35-EcoA3′, was selected that apparently had a deletion of approximately 7 kb of the 5′ end. It did contain the SalI site at 9.4 kb in Ad35 wt DNA and approximately 1.5 kb of sequences upstream. Using this SalI site and the unique NdeI site in the pBr322 backbone, this clone is extended to the 5′ end by insertion of an approximately 5 kb Ad35 fragment 5′ from the first SalI in Ad35 in such a way that a NotI restriction site is created at the 5′ end of the Ad35 by insertion of a linker. This clone, designated pBr/Ad35.pIX-EcoA, does not contain the left-end sequences (ITR, packaging sequences and E1) and at the 3′ end, it has approximately 3.5 kb overlap with clone pBr/Ad35.Sal2-rITR.

To create an adapter plasmid, Ad35 was digested with SalI and the left-end B fragment of ˜9.4 kb was isolated. pBr322 was digested with EcoRV and SalI, isolated from gel and dephosphorylated with Tsap enzyme. Both fragments are ligated and clones with correct insertion and correct sequence of the left ITR are selected. To enable liberation of the left ITR from the vector sequences at the time of virus generation, a NotI site flanking the left ITR is introduced by PCR. From this clone, the E1 sequences are deleted and replaced by a polylinker sequence using PCR. The polylinker sequence is used to introduce an expression cassette for a gene of choice.

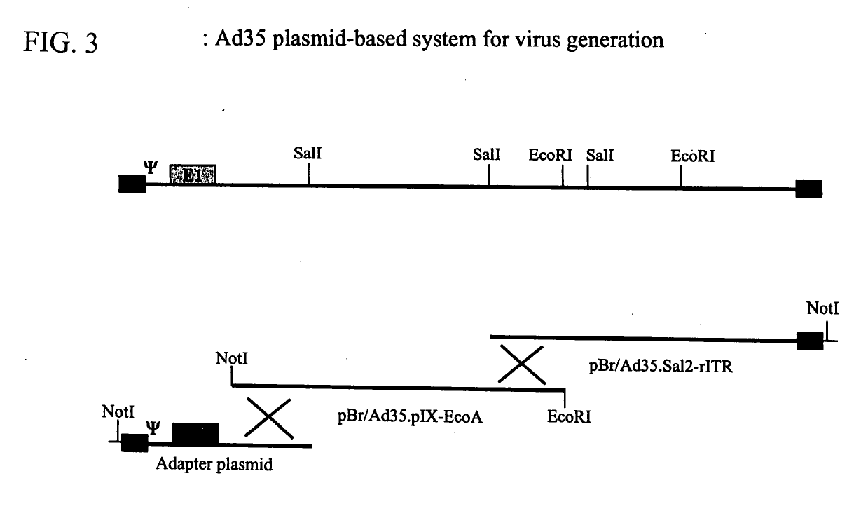

Recombinant Ad35 clones are generated by transfection of PER.C6 cells with the adapter plasmid, pBr/Ad35.pIX-EcoA and pBr/Ad35.Sal2-rITR as shown in FIG. 3. Homologous recombination gives rise to recombinant viruses.

### Example 5

The Prevalence of Neutralizing Activity (NA) to Ad35 is Low in Human Sera from Different Geographic Locations

In Example 1 the analysis of neutralizing activity (“NA”) in human sera from one location in Belgium was described. Strikingly, of a panel of 44 adenovirus serotypes tested, one serotype, Ad35, was not neutralized in any of the 100 sera assayed. In addition, a few serotypes, Ad26, Ad34 and Ad48, were found to be neutralized in 8%, or less, of the sera tested. This analysis was further extended to other serotypes of adenovirus not previously tested and, using a selection of serotypes from the first screen, was also extended to sera from different geographic locations.

Hereto, adenoviruses were propagated, purified and tested for neutralization in the CPE-inhibition assay as described in Example 1. Using the sera from the same batch as in Example 1, adenovirus serotypes 7B, 11, 14, 18 and 44/1876 were tested for neutralization. These viruses were found to be neutralized in, respectively, 59, 13, 30, 98 and 54% of the sera. Thus, of this series, Ad11 is neutralized with a relatively low frequency.

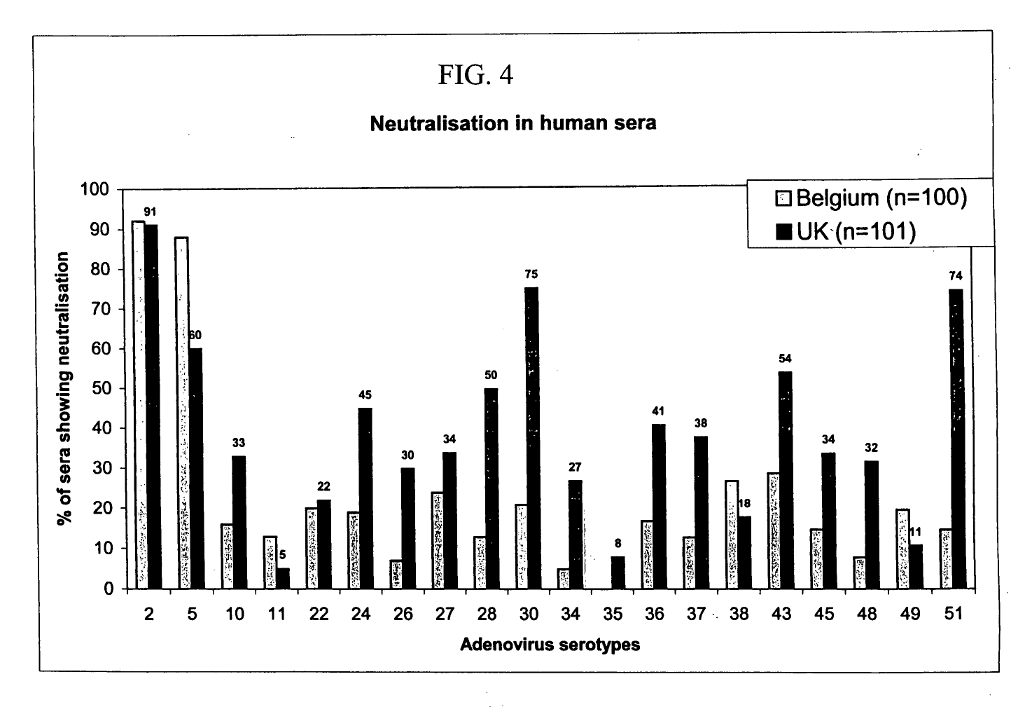

Since it is known that the frequency of isolation of adenovirus serotypes from human tissue, as well as the prevalence of NA to adenovirus serotypes, may differ on different geographic locations, we further tested a selection of the adenovirus serotypes against sera from different places. Human sera were obtained from two additional places in Europe (Bristol, UK and Leiden, NL) and from two places in the United States (Stanford, Calif. and Great Neck, N.Y.). Adenoviruses that were found to be neutralized in 20% or less of the sera in the first screen, as well as Ad2, Ad5, Ad27, Ad30, Ad38, Ad43, were tested for neutralization in sera from the UK. The results of these experiments are presented in FIG. 4. Adenovirus serotypes 2 and 5 were again neutralized in a high percentage of human sera. Furthermore, some of the serotypes that were neutralized in a low percentage of sera in the first screen are neutralized in a higher percentage of sera from the UK, e.g., Ad26 (7% vs. 30%), Ad28 (13% vs. 50%), Ad34 (5% vs. 27%) and Ad48 (8% vs. 32%). Neutralizing activity against Ad11 and Ad49 that were found in a relatively low percentage of sera in the first screen, are found in an even lower percentage of sera in this second screen (13% vs. 5% and 20% vs. 11%, respectively). Serotype Ad35 that was not neutralized in any of the sera in the first screen, was now found to be neutralized in a low percentage (8%) of sera from the UK. The prevalence of NA in human sera from the UK is the lowest to serotypes Ad11 and Ad35.

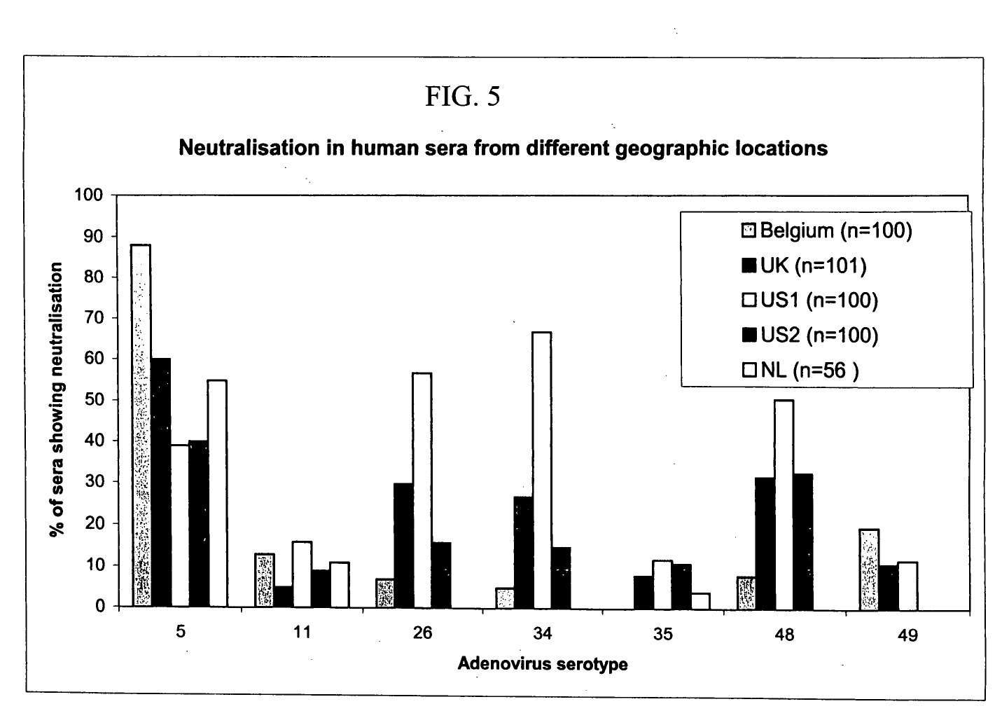

For further analysis, sera was obtained from two locations in the US (Stanford, Calif. and Great Neck, N.Y.) and from The Netherlands (Leiden). FIG. 5 presents an overview of data obtained with these sera and the previous data. Not all viruses were tested in all sera, except for Ad5, Ad11 and Ad35. The overall conclusion from this comprehensive screen of human sera is that the prevalence of neutralizing activity to Ad35 is the lowest of all serotypes throughout the western countries: on average, 7% of the human sera contain neutralizing activity (five different locations). Another B-group adenovirus, Ad11, is also neutralized in a low percentage of human sera (average 11% in sera from five different locations). Adenovirus type 5 is neutralized in 56% of the human sera obtained from five different locations. Although not tested in all sera, D-group serotype 49 is also neutralized with relatively low frequency in samples from Europe and from one location in the U.S. (average 14%).

In the herein-described neutralization experiments, a serum is judged non-neutralizing when, in the well with the highest serum concentration, the maximum protection of CPE is 40% compared to the controls without serum. The protection is calculated as follows:

$$\% protection = \frac{OD corresponding well - OD virus control}{OD non \text{-} infected control - OD virus control} \times 100 \%$$

As described in Example 1, the serum is plated in five different dilutions ranging from 4× to 64× diluted. Therefore, it is possible to distinguish between low titers (i.e., neutralization only in the highest serum concentrations) and high titers of NA (i.e., also neutralization in wells with the lowest serum concentration). Of the human sera used in our screen that were found to contain neutralizing activity to Ad5, 70% turned out to have high titers, whereas of the sera that contained NA to Ad35, only 15% had high titers. Of the sera that were positive for NA to Ad11, only 8% had high titers. For Ad49, this was 5%. Therefore, not only is the frequency of NA to Ad35, Ad11 and Ad49 much lower as compared to Ad5, but of the sera that do contain NA to these viruses, the vast majority has low titers. Adenoviral vectors based on Ad11, Ad35 or Ad49 have, therefore, a clear advantage over Ad5-based vectors when used as gene therapy vehicles or vaccination vectors in vivo or in any application where infection efficiency is hampered by neutralizing activity.

In the following examples, the construction of a vector system for the generation of safe, RCA-free Ad35-based vectors is described.

### Example 6

Sequence of the Human Adenovirus Type 35

Ad35 viruses were propagated on PER.C6 cells and DNA was isolated as described in Example 4. The total sequence was generated by Qiagen Sequence Services (Qiagen GmbH, Germany). Total viral DNA was sheared by sonification and the ends of the DNA were made blunt by T4 DNA polymerase. Sheared blunt fragments were size fractionated on agarose gels and gel slices corresponding to DNA fragments of 1.8 to 2.2 kb were obtained. DNA was purified from the gel slices by the QIAquick gel extraction protocol and subcloned into a shotgun library of pUC19 plasmid cloning vectors. An array of clones in 96-well plates covering the target DNA eight (±two) times was used to generate the total sequence. Sequencing was performed on Perkin-Elmer 9700 thermocyclers using Big Dye Terminator chemistry and AmpliTaq FS DNA polymerase followed by purification of sequencing reactions using QIAGEN DyeEx 96 technology. Sequencing reaction products were then subjected to automated separation and detection of fragments on ABI 377 XL 96 lane sequencers. Initial sequence results were used to generate a contig sequence and gaps were filled in by primer walking reads on the target DNA or by direct sequencing of PCR products. The ends of the virus turned out to be absent in the shotgun library, most probably due to cloning difficulties resulting from the amino acids of pTP that remain bound to the ITR sequences after proteinase K digestion of the viral DNA. Additional sequence runs on viral DNA solved most of the sequence in those regions, however it was difficult to obtain a clear sequence of the most terminal nucleotides. At the 5′ end, the sequence portion obtained was 5′-CCA ATA ATA TAC CT-3′ (SEQ ID NO:38) while at the 3′ end, the obtained sequence portion was 5′-AGG TAT ATT ATT GAT GAT GGG-3′ (SEQ ID NO:39). Most human adenoviruses have a terminal sequence 5′-CAT CAT CAA TAA TAT ACC-3′ (SEQ ID NO:40). In addition, a clone representing the 3′ end of the Ad35 DNA obtained after cloning the terminal 7 kb Ad35 EcoRI fragment into pBr322 (see, Example 4) also turned out to have the typical CATCATCAATAAT . . . sequence as seen in SEQ ID NO:40. Therefore, Ad35 may have the typical end sequence and the differences obtained in sequencing directly on the viral DNA are due to artifacts correlated with run-off sequence runs and the presence of residual amino acids of pTP.

The total sequence of Ad35 with corrected terminal sequences is given in SEQ ID NO:82. Based on sequence homology with Ad5 (Genbank #M72360) and Ad7 (partial sequence Genbank #X03000) and on the location of open reading frames, the organization of the virus is identical to the general organization of most human adenoviruses, especially the subgroup B viruses. The total length of the genome is 34,794 basepairs.

### Example 7

Construction of a Plasmid-Based Vector System to Generate Recombinant Ad35-Based Viruses

A functional plasmid-based vector system to generate recombinant adenoviral vectors comprises the following components:
1. An adapter plasmid comprising a left ITR and packaging sequences derived from Ad35 and at least one restriction site for insertion of a heterologous expression cassette and lacking E1 sequences. Furthermore, the adapter plasmid contains Ad35 sequences 3′ from the E1B-coding region including the pIX promoter and coding sequences enough to mediate homologous recombination of the adapter plasmid with a second nucleic acid molecule.
2. A second nucleic acid molecule, comprising sequences homologous to the adapter plasmid, and Ad35 sequences necessary for the replication and packaging of the recombinant virus, that is early, intermediate and late genes that are not present in the packaging cell.
3. A packaging cell providing at least functional E1 proteins capable of complementing the E1 function of Ad35.

Other methods for the generation of recombinant adenoviruses on complementing packaging cells are known in the art and may be applied to Ad35 viruses without departing from the invention. As an example, the construction of a plasmid-based system, as outlined above, is described in detail below.

Construction of Ad35 Adapter Plasmids

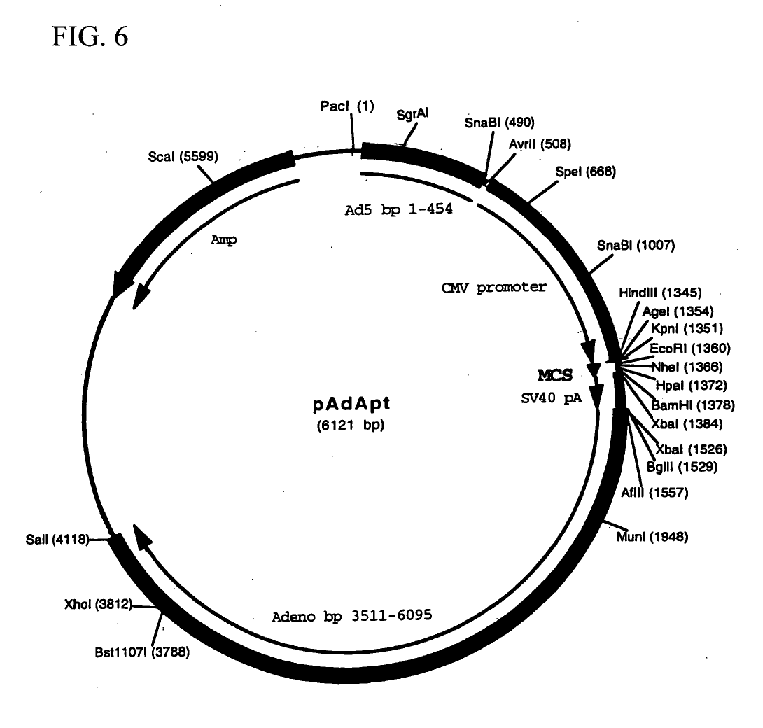

Hereto, the adapter plasmid pAdApt (FIG. 6; described in Example 2) was first modified to obtain adapter plasmids that contain extended polylinkers and that have convenient unique restriction sites flanking the left ITR and the adenovirus sequence at the 3′ end to enable liberation of the adenovirus insert from plasmid vector sequences. Construction of these plasmids is described below in detail:

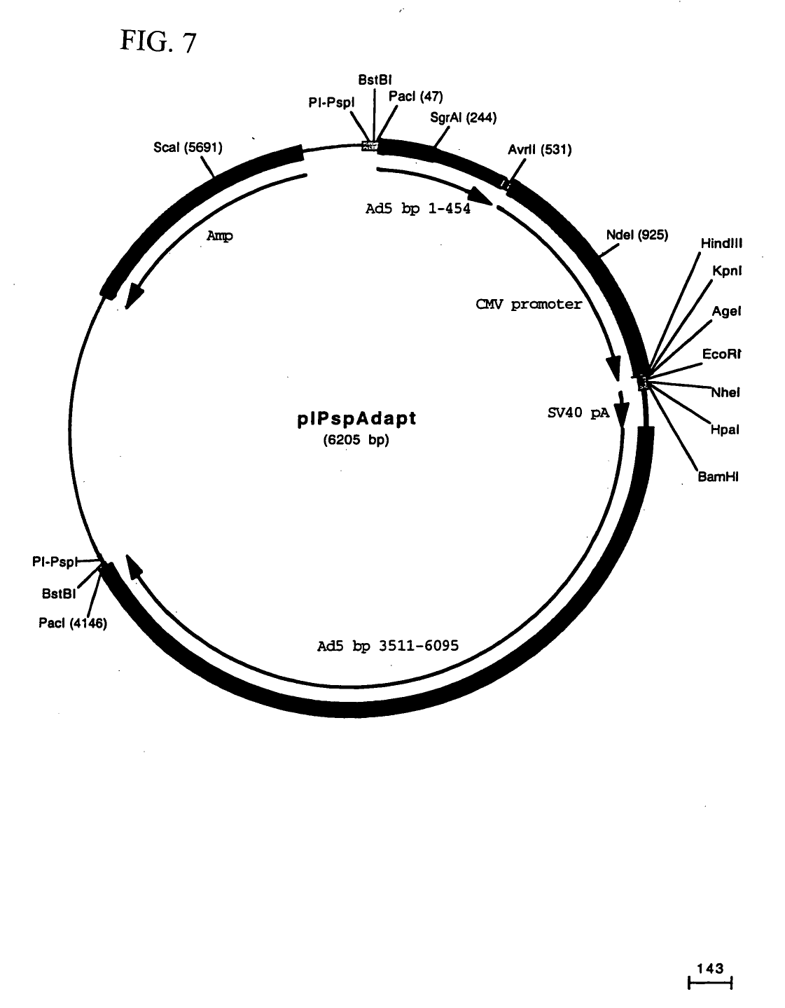

Adapter plasmid pAdApt (Example 2) was digested with SalI and treated with Shrimp Alkaline Phosphatase to reduce religation. A linker, composed of the following two phosphorylated and annealed oligos: ExSalPacF 5′-TCG ATG GCA AAC AGC TAT TAT GGG TAT TAT GGG TTC GAA TTA ATT AA-3′ (SEQ ID NO:41); and ExSalPacR 5′-TCG ATT AAT TAA TTC GAA CCC ATA ATA CCC ATA ATA GCT GTT TGC CA-3′ (SEQ ID NO:42); was directly ligated into the digested construct, thereby replacing the SalI restriction site by Pi-PspI, SwaI and PacI. This construct was designated pADAPT+ExSalPac linker. Furthermore, part of the left ITR of pAdApt was amplified by PCR using the following primers: PCLIPMSF: 5′-CCC CAA TTG GTC GAC CAT CAT CAA TAA TAT ACC TTA TTT TGG-3′ (SEQ ID NO:43) and pCLIPBSRGI: 5′-GCG AAA ATT GTC ACT TCC TGT G-3′ (SEQ ID NO:44). The amplified fragment was digested with MunI and BsrGI and cloned into pAd5/Clip (see, Example 2), which was partially digested with EcoRI and, after purification, digested with BsrGI, thereby re-inserting the left ITR and packaging signal. After restriction enzyme analysis, the construct was digested with ScaI and SgrAI and an 800 bp fragment was isolated from gel and ligated into ScaI/SgrAI-digested pADAPT+ExSalPac linker. The resulting construct, designated pIPspSalAdapt, was digested with SalI, dephosphorylated, and ligated to the phosphorylated ExSalPacF/ExSalPacR double-stranded linker previously mentioned. A clone in which the PacI site was closest to the ITR was identified by restriction analysis and sequences were confirmed by sequence analysis. This novel pAdApt construct, termed pIPspAdapt (FIG. 7) thus harbors two ExSalPac linkers containing recognition sequences for PacI, PI-PspI and BstBI, which surround the adenoviral part of the adenoviral adapter construct, and which can be used to linearize the plasmid DNA prior to co-transfection with adenoviral helper fragments.

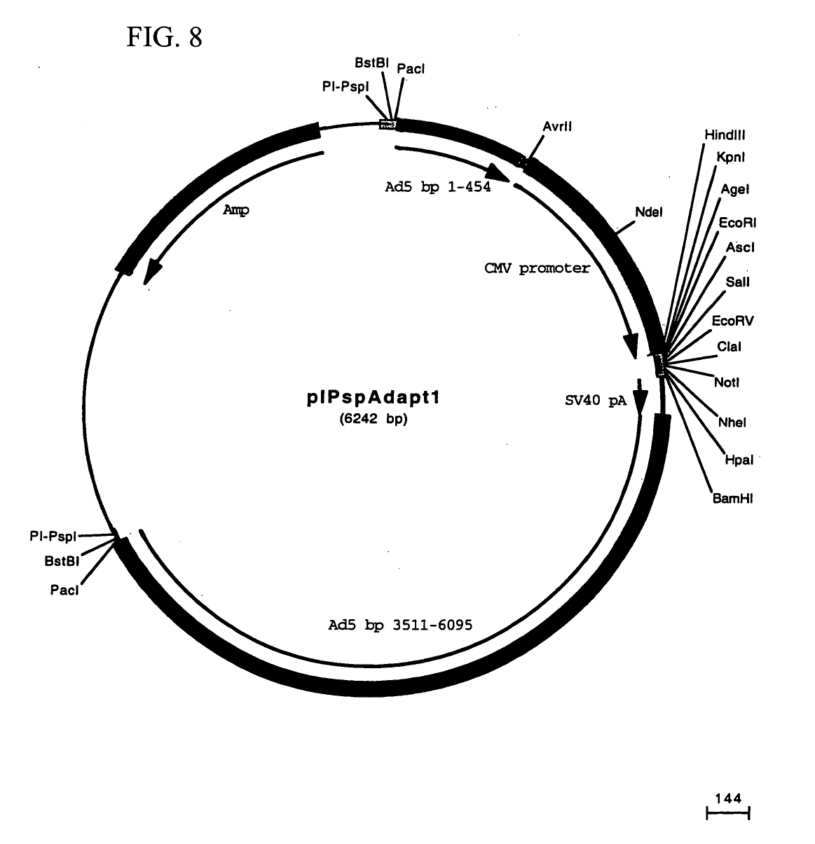

In order to further increase transgene cloning permutations, a number of polylinker variants were constructed based on pIPspAdapt. For this purpose, pIPspAdapt was first digested with EcoRI and dephosphorylated. A linker composed of the following two phosphorylated and annealed oligos: Ecolinker+: 5′-AAT TCG GCG CGC CGT CGA CGA TAT CGA TAG CGG CCG C-3′ (SEQ ID NO:45) and Ecolinker−: 5′-AAT TGC GGC CGC TAT CGA TAT CGT CGA CGG CGC GCC G-3′ (SEQ ID NO:46) was ligated into this construct, thereby creating restriction sites for AscI, SalI, EcoRV, ClaI and NotI. Both orientations of this linker were obtained, and sequences were confirmed by restriction analysis and sequence analysis. The plasmid containing the polylinker in the order 5′ HindIII, KpnI, AgeI, EcoRI, AscI, SalI, EcoRV, ClaI, NotI, NheI, HpaI, BamHI and XbaI was termed pIPspAdapt1 (FIG. 8) while the plasmid containing the polylinker in the order HindIII, KpnI, AgeI, NotI, ClaI, EcoRV, SalI, AscI, EcoRI, NheI, HpaI, BamHI and XbaI was termed pIPspAdapt2.

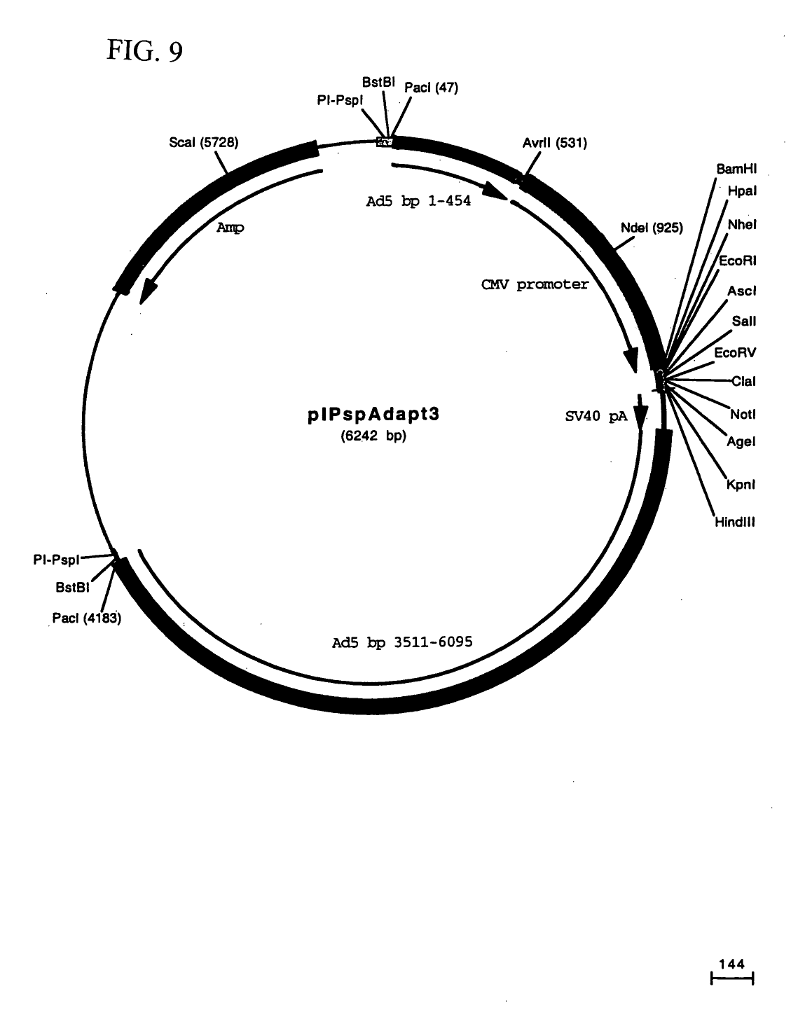

To facilitate the cloning of other sense or antisense constructs, a linker composed of the following two oligonucleotides was designed to reverse the polylinker of pIPspAdapt: HindXba+5′-AGC TCT AGA GGA TCC GTT AAC GCT AGC GAA TTC ACC GGT ACC AAG CTT A-3′ (SEQ ID NO:47); HindXba−5′-CTA GTA AGC TTG GTA CCG GTG AAT TCG CTA GCG TTA ACG GAT CCT CTA G-3′ (SEQ ID NO:48). This linker was ligated into HindIII/XbaI-digested pIPspAdapt and the correct construct was isolated. Confirmation was done by restriction enzyme analysis and sequencing. This new construct, pIPspAdaptA, was digested with EcoRI and the previously mentioned Ecolinker was ligated into this construct. Both orientations of this linker were obtained, resulting in pIPspAdapt3 (FIG. 9), which contains the polylinker in the order XbaI, BamHI, HpaI, NheI, EcoRI, AscI, SalI, EcoRV, ClaI, NotI, AgeI, KpnI and HindIII. All sequences were confirmed by restriction enzyme analysis and sequencing.

Adapter plasmids based on Ad35 were then constructed as follows:

The left ITR and packaging sequence corresponding to Ad35 wt sequences nucleotides 1 to 464 (SEQ ID NO:82) were amplified by PCR on wtAd35 DNA using the following primers: Primer 35F1: 5′-CGG AAT TCT TAA TTA ATC GAC ATC ATC AAT AAT ATA CCT TAT AG-3′ (SEQ ID NO:49) and Primer 35R2: 5′-GGT GGT CCT AGG CTG ACA CCT ACG TAA AAA CAG-3′ (SEQ ID NO:50).

Amplification introduces a PacI site at the 5′ end and an AvrII site at the 3′ end of the sequence. For the amplification, Platinum Pfx DNA polymerase enzyme (LTI) was used according to manufacturer's instructions, but with primers at 0.6 μM and with DMSO added to a final concentration of 3%. Amplification program was as follows: 2 minutes at 94° C. (30 seconds at 94° C., 30 seconds at 56° C., 1 minute at 68° C.) for 30 cycles, followed by 10 minutes at 68° C.

The PCR product was purified using a PVR purification kit (LTI) according to the manufacturer's instructions, and digested with PacI and AvrII. The digested fragment was then purified from gel using the GENECLEAN kit (Bio 101, Inc.). The Ad5-based adapter plasmid pIPspAdApt-3 (FIG. 9) was digested with AvrII and then partially with PacI and the 5762 bp fragment was isolated in an LMP agarose gel slice and ligated with the above-mentioned PCR fragment digested with the same enzymes and transformed into electrocompetent DH10B cells (LTI). The resulting clone is designated pIPspAdApt3-Ad351ITR.

In parallel, a second piece of Ad35 DNA was amplified using the following primers: 35F3: 5′-TGG TGG AGA TCT GGT GAG TAT TGG GAA AAC-3′ (SEQ ID NO:51) and 35R4: 5′-CGG AAT TCT TAA TTA AGG GAA ATG CAA ATC TGT GAG G-3′ (SEQ ID NO:52).

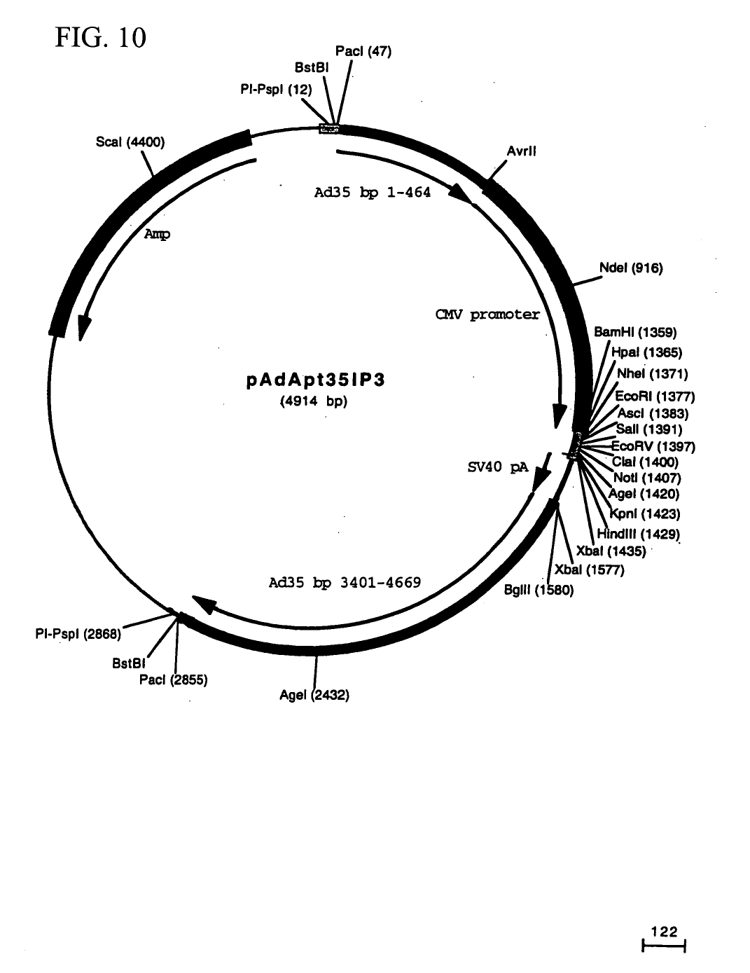

The sequence of this fragment corresponds to nucleotides 3401 to 4669 of wtAd35 (SEQ ID NO:82) and contains 1.3kb of sequences starting directly 3′ from the E1B 55 k-coding sequence. Amplification and purification were done as previously described herein for the fragment containing the left ITR and packaging sequence. The PCR fragment was then digested with PacI and subcloned into pNEB193 vector (New England Biolabs) digested with SmaI and PacI. The integrity of the sequence of the resulting clone was checked by sequence analysis. pNEB/Ad35pF3R4 was then digested with BglII and PacI and the Ad35 insert was isolated from gel using the QIAExII kit (Qiagen). pIPspAdApt3-Ad351ITR was digested with BglII and then partially with PacI. The 3624 bp fragment (containing vector sequences, the Ad35 ITR and packaging sequences as well as the CMV promoter, multiple cloning region and polyA signal) was also isolated using the QIAExII kit (Qiagen). Both fragments were ligated and transformed into competent DH10B cells (LTI). The resulting clone, pAdApt35IP3 (FIG. 10), has the expression cassette from pIPspAdApt3 but contains the Ad35 left ITR and packaging sequences and a second fragment corresponding to nucleotides 3401 to 4669 from Ad35. A second version of the Ad35 adapter plasmid having the multiple cloning site in the opposite orientation was made as follows:

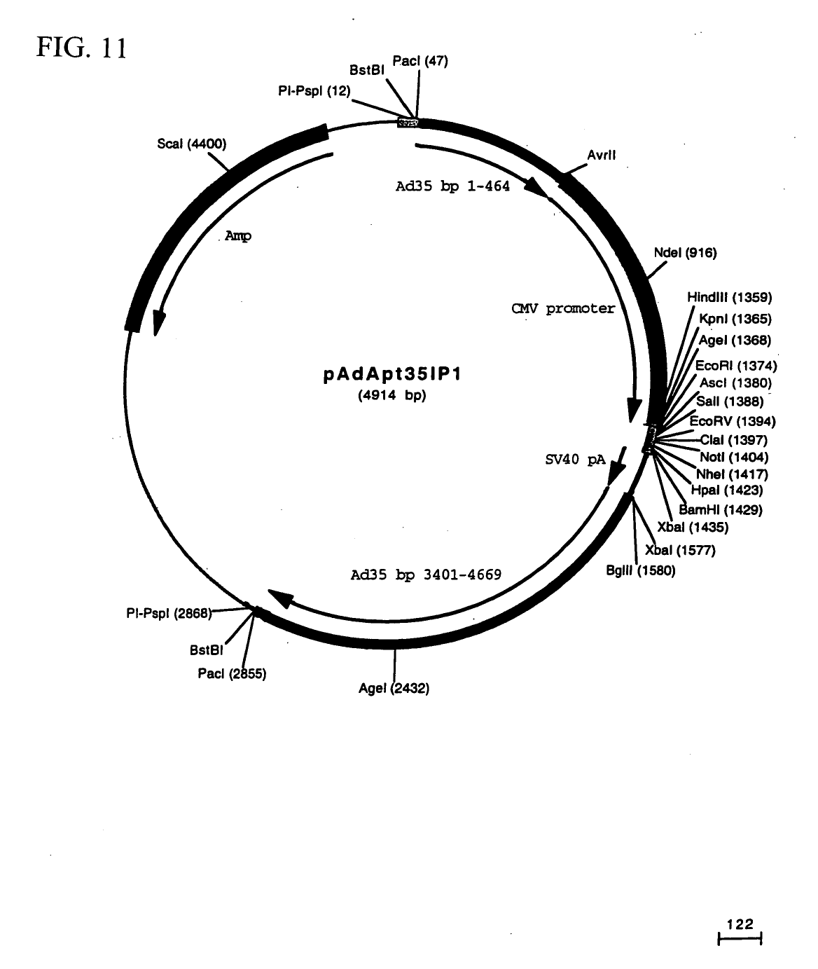

pIPspAdapt1 (FIG. 8) was digested with NdeI and BglII and the 0.7 kbp band containing part of the CMV promoter, the MCS and SV40 polyA was isolated and inserted in the corresponding sites of pAdApt35IP3 generating pAdApt35IP1 (FIG. 11).

pAdApt35.LacZ and pAdApt35.Luc adapter plasmids were then generated by inserting the transgenes from pcDNA.LacZ (digested with KpnI and BamHI) and pAdApt.Luc (digested with HindIII and BamHI) into the corresponding sites in pAdApt35IP1. The generation of pcDNA.LacZ and pAdApt.Luc is described in International Patent Application WO99/55132.

Construction of Cosmid pWE.Ad35.pXI-rITR

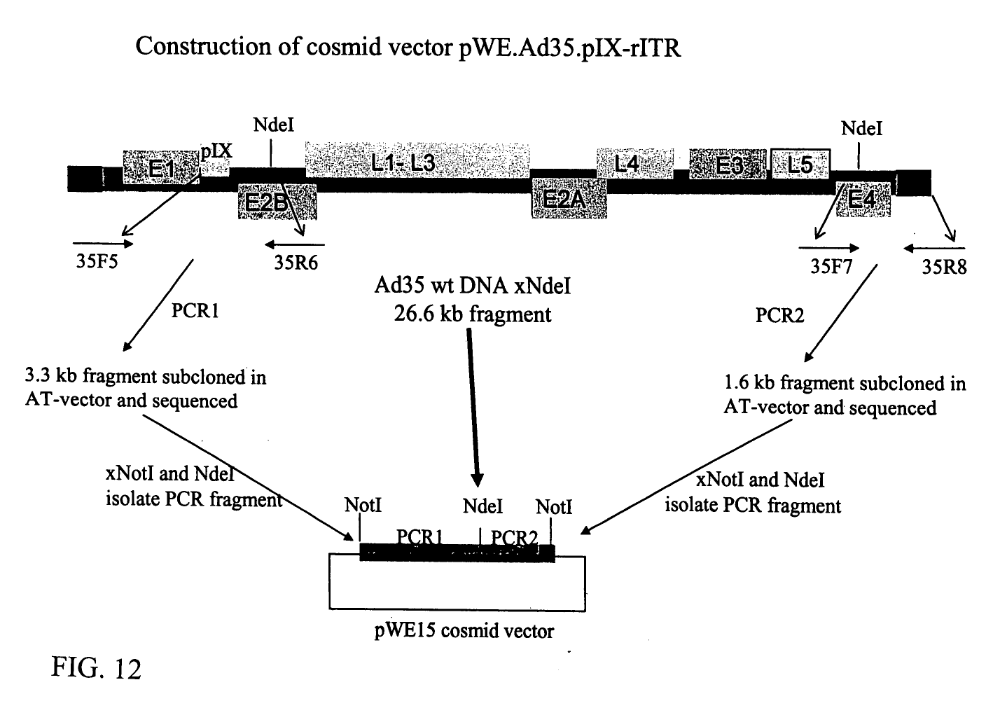

FIG. 12 presents the various steps undertaken to construct the cosmid clone containing Ad35 sequences from bp 3401 to 34794 (end of the right ITR) that are described in detail below.

A first PCR fragment (pIX-NdeI) was generated using the following primer set:

35F5:

(SEQ ID NO:53)

5′-CGG AAT TCG CGG CCG CGG TGA GTA TTG GGA AAA C-
3′
and
35R6:

(SEQ ID NO:54)

5′-CGC CAG ATC GTC TAC AGA ACA G-3′.

DNA polymerase Pwo (Roche) was used according to the manufacturer's instructions, however, with an end concentration of 0.6 μM of both primers and using 50 ngr wt Ad35 DNA as template. Amplification was done as follows: 2 minutes at 94° C., 30 cycles of 30 seconds at 94° C., 30 seconds at 65° C. and 1 minute 45 seconds at 72° C., followed by 8 minutes at 68° C. To enable cloning in the TA cloning vector PCR2.1, a last incubation with 1 unit superTaq polymerase (HT Biotechnology LTD) for 10 minutes at 72° C. was performed.

The 3370 bp-amplified fragment contains Ad35 sequences from bp 3401 to 6772 with a NotI site added to the 5′ end. Fragments were purified using the PCR purification kit (LTI).

A second PCR fragment (NdeI-rITR) was generated using the following primers:

35F7:

(SEQ ID NO:55)

5′-GAA TGC TGG CTT CAG TTG TAA TC-3′
and
35R8:

(SEQ ID NO:56)

5′-CGG AAT TCG CGG CCG CAT TTA AAT CAT CAT CAA TAA
TAT ACC-3′.

Amplification was done with pfx DNA polymerase (LTI) according to manufacturer's instructins but with 0.6 μM of both primers and 3% DMSO using 10 ngr. of wtAd35 DNA as template. The program was as follows: 3 minutes at 94° C. and 5 cycle of 30 seconds at 94° C., 45 seconds at 40° C., 2 minutes 45 seconds at 68° C. followed by 25 cycles of 30 seconds at 94° C., 30 seconds at 60° C., 2 minutes 45 seconds at 68° C. To enable cloning in the TA-cloning vector PCR2.1, a last incubation with 1 unit superTaq polymerase for 10 minutes at 72° C. was performed. The 1.6 kb amplified fragment ranging from nucleotides 33178 to the end of the right ITR of Ad35, was purified using the PCR purification kit (LTI).

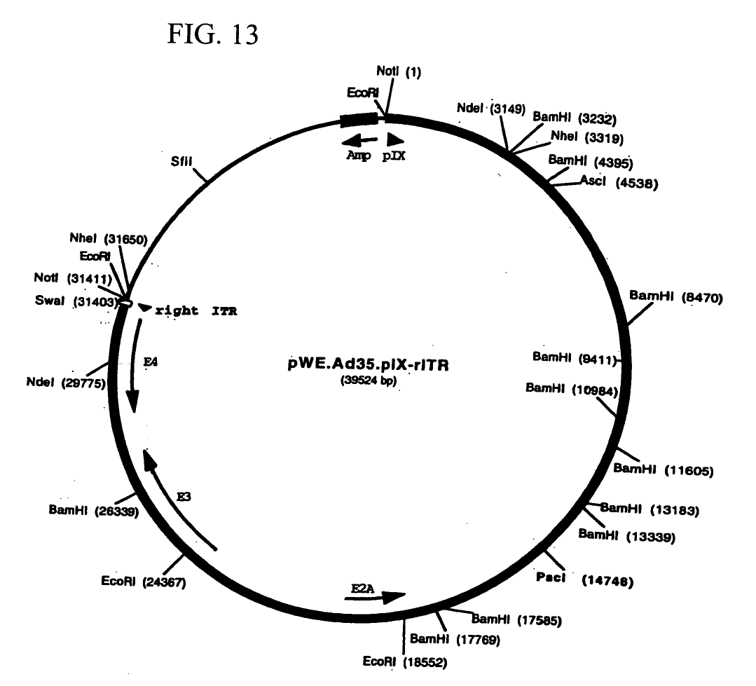

Both purified PCR fragments were ligated into the PCR2.1 vector of the TA-cloning kit (Invitrogen) and transformed into STBL-2-competent cells (LTI). Clones containing the expected insert were sequenced to confirm correct amplification. Next, both fragments were excised from the vector by digestion with NotI and NdeI and purified from gel using the GENECLEAN kit (BIO 101, Inc.). Cosmid vector pWE15 (Clontech) was digested with NotI, dephosphorylated and also purified from gel. These three fragments were ligated and transformed into STBL2-competent cells (LTI). One of the correct clones that contained both PCR fragments was then digested with NdeI, and the linear fragment was purified from gel using the GENECLEAN kit. Ad35 wt DNA was digested with NdeI and the 26.6 kb fragment was purified from LMP gel using agarase enzyme (Roche) according to the manufacturer's instructions. These fragments were ligated together and packaged using λ phage packaging extracts (Stratagene) according to the manufacturer's protocol. After infection into STBL-2 cells, colonies were grown on plates and analyzed for presence of the complete insert. One clone with the large fragment inserted in the correct orientation and having the correct restriction patterns after independent digestions with three enzymes (NcoI, PvuII and ScaI) was selected. This clone is designated pWE.Ad35.pIX-rITR. It contains the Ad35 sequences from bp 3401 to the end and is flanked by NotI sites (FIG. 13).

Generation of Ad35-Based Recombinant Viruses on PER. C6

Wild-type Ad35 virus can be grown on PER.C6 packaging cells to very high titers. However, whether the Ad5-E1 region that is present in PER.C6 is able to complement E1-deleted Ad35-recombinant viruses is unknown. To test this, PER.C6 cells were co-transfected with the above-described adapter plasmid pAdApt35.LacZ and the large backbone fragment pWE.Ad35.pIX-rITR. First, pAdApt35.LacZ was digested with PacI and pWE.Ad35.pIX-rITR was digested with NotI. Without further purification, 4 μgr of each construct was mixed with DMEM (LTI) and transfected into PER.C6 cells, seeded at a density of 5×10⁶ cells in a T25 flask the day before, using Lipofectamin (LTI) according to the manufacturer's instructions. As a positive control, 6 μgr of PacI-digested pWE.Ad35.pIX-rITR DNA was co-transfected with a 6.7 kb NheI fragment isolated from Ad35 wt DNA containing the left end of the viral genome including the E1 region. The next day, medium (DMEM with 10% FBS and 10 mM MgCl₂) was refreshed and cells were further incubated. At day 2 following the transfection, cells were trypsinized and transferred to T80 flasks. The positive control flask showed CPE at five days following transfection, showing that the pWE.Ad35.pIX-rITR construct is functional at least in the presence of Ad35-E1 proteins. The transfection with the Ad35 LacZ adapter plasmid and pWE.Ad35.pIX-rITR did not give rise to CPE. These cells were harvested in the medium at day 10 and freeze/thawed once to release virus from the cells. 4 ml of the harvested material was added to a T80 flask with PER.C6 cells (at 80% confluency) and incubated for another five days. This harvest/re-infection was repeated for two times but there was no evidence for virus-associated CPE.

From this experiment, it seems that the Ad5-E1 proteins are not, or not well enough, capable of complementing Ad35-recombinant viruses; however, it may be that the sequence overlap of the adapter plasmid and the pWE.Ad35.pIX-rITR backbone plasmid is not large enough to efficiently recombine and give rise to a recombinant virus genome. The positive control transfection was done with a 6.7 kb left-end fragment and, therefore, the sequence overlap was about 3.5 kb. The adapter plasmid and the pWE.Ad35.pIX-rITR fragment have a sequence overlap of 1.3 kb. To check whether the sequence overlap of 1.3 kb is too small for efficient homologous recombination, a co-transfection was done with PacI-digested pWE.Ad35.pIX-rITR and a PCR fragment of Ad35 wt DNA generated with the above-mentioned 35F1 and 35R4 using the same procedures as previously described herein. The PCR fragment thus contains left-end sequences up to bp 4669 and, therefore, has the same overlap sequences with pWE.Ad35.pIX-rITR as the adapter plasmid pAdApt35.LacZ, but has Ad35 E1 sequences. Following PCR column purification, the DNA was digested with SalI to remove possible intact template sequences. A transfection with the digested PCR product alone served as a negative control. Four days after the transfection, CPE occurred in the cells transfected with the PCR product and the Ad35 pIX-rITR fragment and not in the negative control. This result shows that a 1.3 kb overlapping sequence is sufficient to generate viruses in the presence of Ad35 E1 proteins. From these experiments, we conclude that the presence of at least one of the Ad35.E1 proteins is necessary to generate recombinant Ad35-based vectors from plasmid DNA on Ad5-complementing cell lines.

### Example 8

Construction of Ad35.E1 Expression Plasmids

Since Ad5-E1 proteins in PER.C6 are incapable of complementing Ad35-recombinant viruses efficiently, Ad35 E1 proteins have to be expressed in Ad5-complementing cells (e.g., PER.C6). Otherwise, a new packaging cell line expressing Ad35 E1 proteins has to be made, starting from either diploid primary human cells or established cell lines not expressing adenovirus E1 proteins. To address the first possibility, the Ad35 E1 region was cloned in expression plasmids as described below.

First, the Ad35 E1 region from bp 468 to bp 3400 was amplified from wtAd35 DNA using the following primer set: 35F11: 5′-GGG GTA CCG AAT TCT CGC TAG GGT ATT TAT ACC-3′ (SEQ ID NO:57) and 35F10: 5′-GCT CTA GAC CTG CAG GTT AGT CAG TTT CTT CTC CAC TG-3′ (SEQ ID NO:58). This PCR introduces a KpnI and EcoRI site at the 5′ end and a SbfI and XbaI site at the 3′ end.

Amplification on 5 ng template DNA was done with Pwo DNA polymerase (Roche) using the manufacturer's instructions, however, with both primers at a final concentration of 0.6 μM. The program was as follows: 2 minutes at 94° C., 5 cycles of 30 seconds at 94° C., 30 seconds at 56° C. and 2 minutes at 72° C., followed by 25 cycles of 30 seconds at 94° C., 30 seconds at 60°C. and 2 minutes at 72° C., followed by 10 minutes at 72° C. PCR product was purified by a PCR purification kit (LTI) and digested with KpnI and XbaI. The digested PCR fragment was then ligated to the expression vector pRSVhbvNeo (see below) also digested with KpnI and XbaI. Ligations were transformed into competent STBL-2 cells (LTI) according to the manufacturer's instructions and colonies were analyzed for the correct insertion of Ad35E1 sequences into the polylinker in between the RSV promoter and HBV polyA.

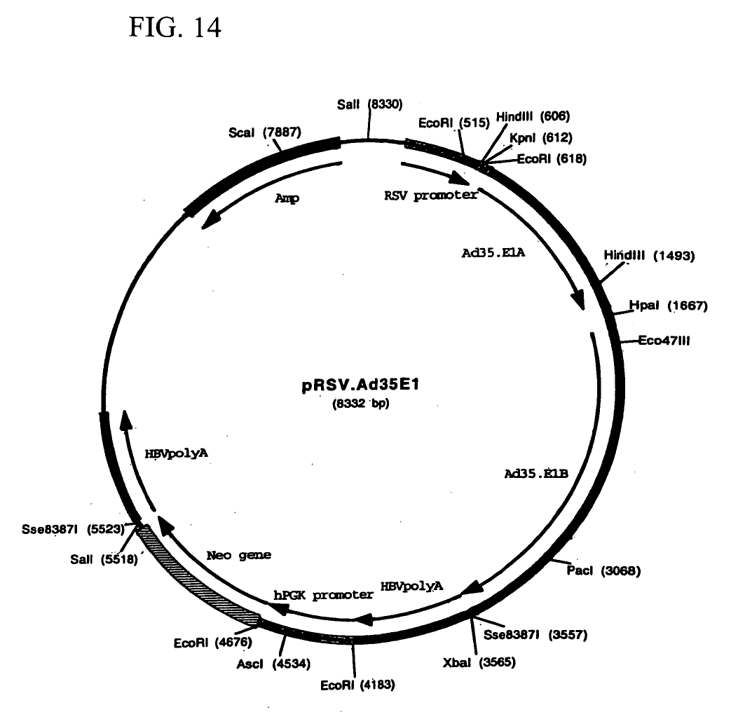

The resulting clone was designated pRSV.Ad35-E1 (FIG. 14). The Ad35 sequences in pRSV.Ad35-E1 were checked by sequence analysis.

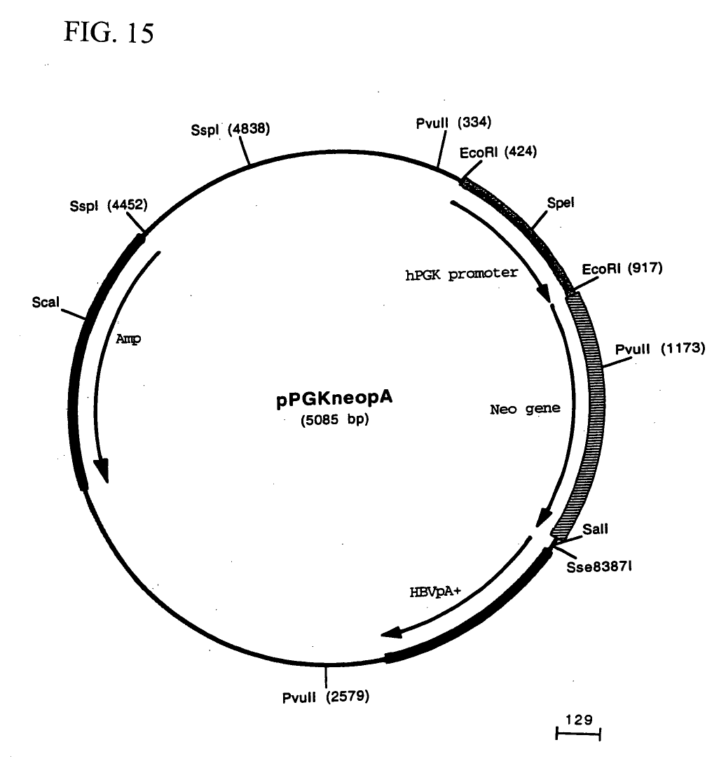

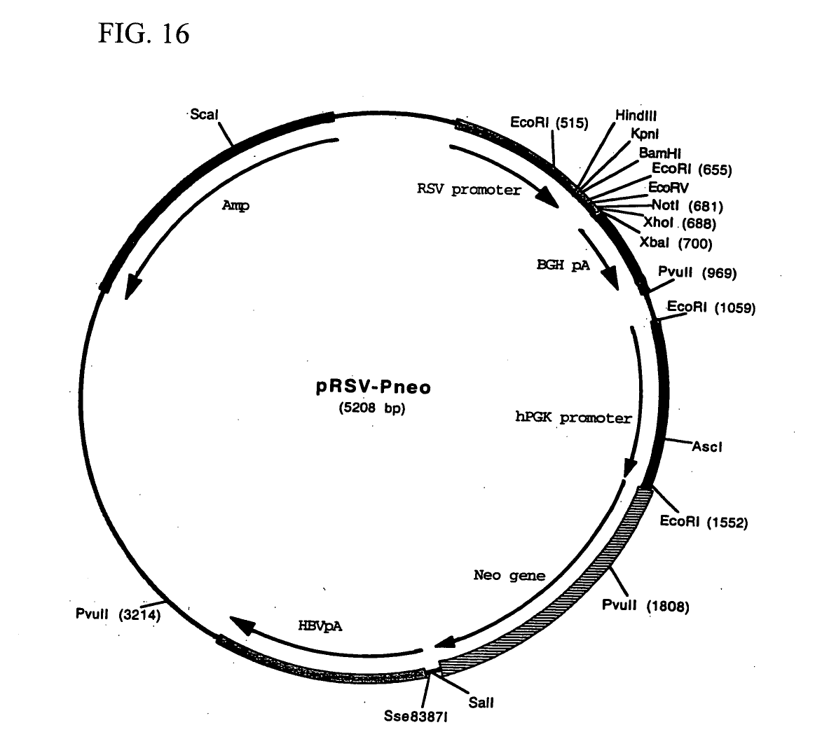

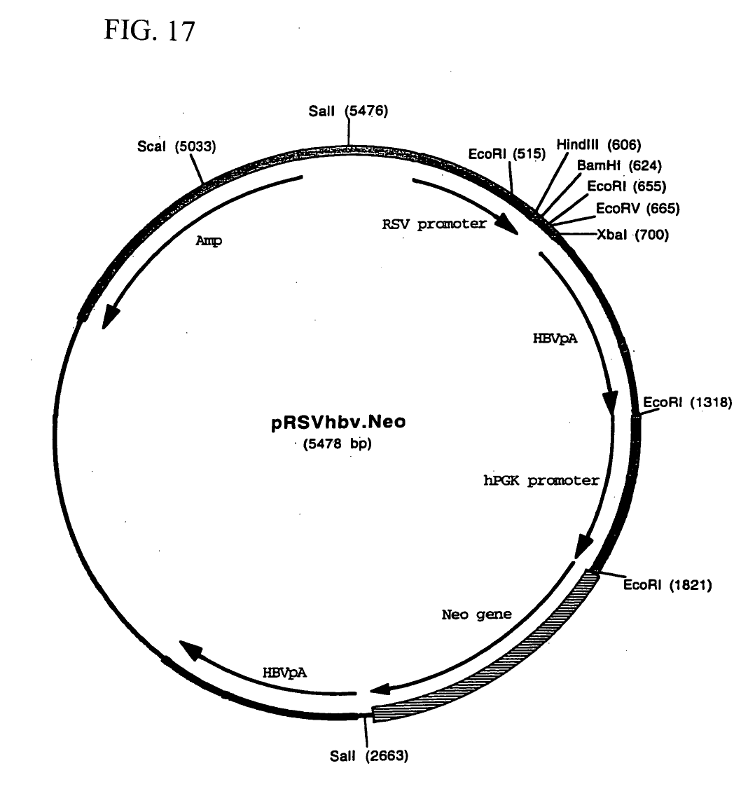

pRSVhbvNeo was generated as follows: pRc-RSV (Invitrogen) was digested with PvuII, dephosphorylated with TSAP enzyme (LTI), and the 3 kb vector fragment was isolated in low-melting point agarose (LMP). Plasmid pPGKneopA (FIG. 15; described in International Patent Application WO96/35798) was digested with SspI completely to linearize the plasmid and facilitate partial digestion with PvuII. Following the partial digestion with PvuII, the resulting fragments were separated on a LMP agarose gel and the 2245 bp PvuII fragment containing the PGK promoter, neomycin-resistance gene and HBVpolyA, was isolated. Both isolated fragments were ligated to give the expression vector pRSV-pNeo that now has the original SV40prom-neo-SV40polyA expression cassette replaced by a PGKprom-neo-HBVpolyA cassette (FIG. 16). This plasmid was further modified to replace the BGHpA with the HBVpA as follows: pRSVpNeo was linearized with ScaI and further digested with XbaI. The 1145 bp fragment, containing part of the Amp gene and the RSV promoter sequences and polylinker sequence, was isolated from gel using the GeneClean kit (Bio Inc. 101). Next, pRSVpNeo was linearized with Scal and further digested with EcoRI partially and the 3704 bp fragment containing the PGKneo cassette and the vector sequences were isolated from gel as above. A third fragment, containing the HBV polyA sequence flanked by XbaI and EcoRI at the 5′ and 3′ ends, respectively, was then generated by PCR amplification on pRSVpNeo using the following primer set: HBV-F: 5′-GGC TCT AGA GAT CCT TCG CGG GAC GTC-3′ (SEQ ID NO:59) and HBV-R: 5′-GGC GAA TTC ACT GCC TTC CAC CAA GC-3′ (SEQ ID NO:60). Amplification was done with Elongase enzyme (LTI) according to the manufacturer's instructions with the following conditions: 30 seconds at 94° C., then 5 cycles of 45 seconds at 94° C., 1 minute at 42° C. and 1 minute 68° C., followed by 30 cycles of 45 seconds at 84° C., 1 minute at 65° C. and 1 minute at 68° C., followed by 10 minutes at 68° C. The 625 bp PCR fragment was then purified using the Qiaquick PCR purification kit, digested with EcoRI and XbaI and purified from gel using the GENECLEAN kit. The three isolated fragments were ligated and transformed into DH5α-competent cells (LTI) to give the construct pRSVhbvNeo (FIG. 17). In this construct, the transcription-regulatory regions of the RSV expression cassette and the neomycin selection marker are modified to reduce overlap with adenoviral vectors that often contain CMV and SV40 transcription-regulatory sequences.

Generation of Ad35-Recombinant Viruses on PER. C6 Cells Co-Transfected with an Ad35-E1 Expression Construct.

PER.C6 cells were seeded at a density of 5×10⁶ cells in a T25 flask and, the next day, transfected with a DNA mixture containing:
1 μg pAdApt35.LacZ digested with PacI
5 μg pRSV.Ad35E1 undigested
2 μg pWE.Ad35.pIX-rITR digested with NotI

Transfection was done using Lipofectamine according to the manufacturer's instructions. Five hours after addition of the transfection mixture to the cells, medium was removed and replaced by fresh medium. After two days, cells were transferred to T80 flasks and further cultured. One week post-transfection, 1 ml of the medium was added to A549 cells and, the following day, cells were stained for LacZ expression. Blue cells were clearly visible after two hours of staining indicating that recombinant LacZ-expressing viruses were produced. The cells were further cultured, but no clear appearance of CPE was noted. However, after 12 days, clumps of cells appeared in the monolayer and 18 days following transfection, cells were detached. Cells and medium were then harvested, freeze-thawed once, and 1 ml of the crude lysate was used to infect PER.C6 cells in a 6-well plate. Two days after infection, cells were stained for LacZ activity. After two hours, 15% of the cells were stained blue. To test for the presence of wt and/or replicating-competent viruses, A549 cells were infected with these viruses and further cultured. No signs of CPE were found indicating the absence of replication-competent viruses. These experiments show that recombinant AdApt35.LacZ viruses were made on PER.C6 cells co-transfected with an Ad35-E1 expression construct.

Ad35-Recombinant Viruses Escape Neutralization in Human Serum Containing Neutralizing Activity to Ad5 Viruses.

The AdApt35.LacZ viruses were then used to investigate infection in the presence of serum that contains neutralizing activity to Ad5 viruses. Purified Ad5-based LacZ virus served as a positive control for NA. Hereto, PER.C6 cells were seeded in a 24-well plate at a density of 2×10⁵ cells/well. The next day, a human serum sample with high neutralizing activity to Ad5 was diluted in culture medium in five steps of five times dilutions. 0.5 ml of diluted serum was then mixed with 4×10⁶ virus particles AdApt5.LacZ virus in 0.5 ml medium and after 30 minutes of incubation at 37° C., 0.5 ml of the mixture was added to PER.C6 cells in duplicate. For the AdApt35.LacZ viruses, 0.5 ml of the diluted serum samples were mixed with 0.5 ml crude lysate containing AdApt35.LacZ virus and after incubation, 0.5 ml of this mixture was added to PER.C6 cells in duplo. Virus samples incubated in medium without serum were used as positive controls for infection. After two hours of infection at 37° C., medium was added to reach a final volume of 1 ml and cells were further incubated. Two days after infection, cells were stained for LacZ activity. The results are shown in Table II. From these results, it is clear that whereas AdApt5.LacZ viruses are efficiently neutralized, AdApt35.LacZ viruses remain infectious irrespective of the presence of human serum. This proves that recombinant Ad35-based viruses escape neutralization in human sera that contain NA to Ad5-based viruses.

### Example 9

An Ad5/Fiber35 Chimeric Vector with Cell Type Specificity for Hemopoietic CD34⁺Lin⁻ Stem Cells

In Example 3, we described the generation of a library of Ad5-based adenoviruses harboring fiber proteins of other serotypes. As a non-limiting example for the use of this library, herein described is the identification of fiber-modified adenoviruses that show improved infection of hemopoietic stem cells.

Cells isolated from human bone marrow, umbilical cord blood, or mobilized peripheral blood carrying the flow cytometric phenotype of being positive for the CD34 antigen and negative for the early differentiation markers CD33, CD38, and CD71 (lin⁻) are commonly referred to as hemopoietic stem cells (HSC). Genetic modification of these cells is of major interest since all hemopoietic lineages are derived from these cells and, therefore, the HSC is a target cell for the treatment of many acquired or congenital human hemopoietic disorders. Examples of diseases that are possibly amenable for genetic modification of HSC include, but are not limited to, Hurlers disease, Hunter's disease, Sanfilippos disease, Morquios disease, Gaucher disease, Farbers disease, Niemann-Pick disease, Krabbe disease, Metachromatic Leucodistrophy, I-cell disease, severe immunodeficiency syndrome, Jak-3 deficiency, Fucosidose deficiency, thallasemia, and erythropoietic porphyria. Besides these hemopoietic disorders, also strategies to prevent or treat acquired immunodeficiency syndrome (“AIDS”) and hemopoietic cancers are based on the genetic modification of HSCs (or cells derived from HSCs such as CD4 positive T-lymphocytes in case of AIDS). The examples listed herein thus aim at introducing DNA into the HSC in order to complement on a genetic level for a gene and protein deficiency. In case of strategies for AIDS or cancer, the DNA to be introduced into the HSC can be anti-viral genes or suicide genes.

Besides the examples listed herein, several other areas exist in which efficient transduction of HSCs using adenoviral vectors can play an important role, for instance, in the field of tissue engineering. In this area, it is important to drive differentiation of HSCs to specific lineages. Some, non-limiting, examples are ex vivo bone formation, cartilage formation, skin formation, as well as the generation of T-cell precursors or endothelial cell precursors. The generation of bone, cartilage or skin in bioreactors can be used for transplantation after bone fractures or spinal cord lesions or severe burn injuries. Naturally, transduced cells can also directly be re-infused into a patient. The formation of large numbers of endothelial cell precursor from HSCs is of interest since these endothelial precursor cells can home, after re-infusion, to sites of cardiovascular injury such as ischemia. Likewise, the formation of large numbers of T-cells from HSCs is of interest since these T-cell precursors can be primed, ex vivo, to eradicate certain targets in the human body after re-infusion of the primed T-cells. Preferred targets in the human body can be tumors or virus-infected cells.

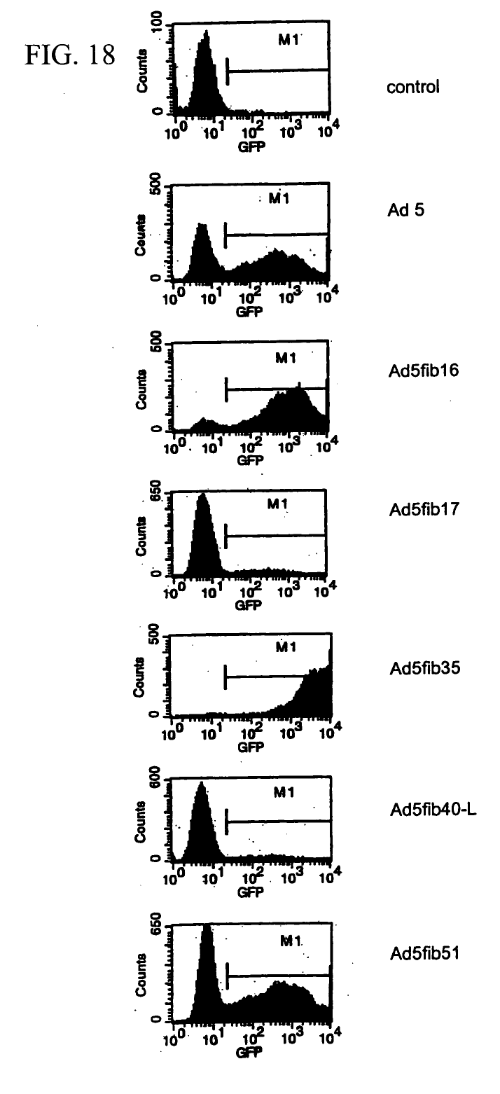

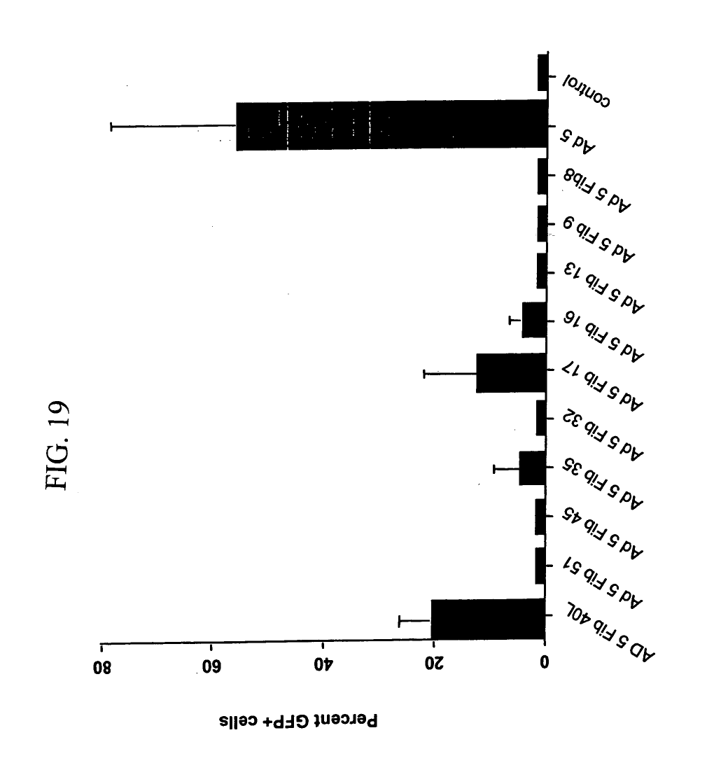

From the herein-described examples, it can be concluded that efficient gene delivery to HSCs is a major interest for the field of gene therapy. Therefore, alteration of the Ad5 host cell range to be able to target HSCs in vitro as well as in vivo is a major interest of the invention. To identify a chimeric adenovirus with preferred infection characteristics for human HSCs, we generated a library of Ad5-based viruses carrying the fiber molecule from alternative adenoviral serotypes (serotypes 8, 9, 13, 16, 17, 32, 35, 45, 40-L, 51). The generation of this fiber-modified library is described in Example 3 hereof. Ad5 was included as a reference. A small panel of this library was tested on human TF-1 (erythroid leukemia, ATCC CRL-2003) whereas all chimeric viruses generated were tested on human primary stroma cells and human HSCs. Human TF-1 cells were routinely maintained in DMEM supplemented with 10% FCS and 50 ng/ ml IL-3 (Sandoz, Basel, Switzerland). Human primary fibroblast-like stroma, isolated from a bone marrow aspirate, is routinely maintained in DMEM/10% FCS. Stroma was seeded at a concentration of 1×10⁵ cells per well of 24-well plates. 24 hours after seeding, cells were exposed for two hours to 1000 virus particles per cell of Ad5, Ad5.Fib16, Ad5.Fib17, Ad5.Fib35, Ad5.Fib40-L, or Ad5.Fib51, all carrying GFP as a marker. After two hours, cells were washed with PBS and reseeded in medium without addition of virus. TF-1 cells were seeded at a concentration of 2×10⁵ cells per well of 24-well plates and were also exposed for two hours to 1000 virus particles of the different chimeric adenoviruses. Virus was removed by washing the cells after the two-hour exposure. Both cell types were harvested 48 hours after virus exposure and analyzed for GFP expression using a flow cytometer. The results on TF-1 cells, shown in FIG. 18, demonstrates that chimeric adenoviruses carrying a fiber from serotypes 16, 35, or 51 (all derived from adenovirus subgroup B) have preferred infection characteristics as compared to Ad5 (subgroup C), Ad5.Fib17 (subgroup D), or Ad5.Fib40-L (subgroup F). Primary human stroma was tested since these cells are commonly used as a “feeder” cell to allow proliferation and maintenance of HSCs under ex vivo culture conditions. In contrast to the transduction of TF-1 cells, none of the fiber chimeric adenoviruses were able to efficiently transduce human primary stroma (FIG. 19). Reasonable infection of human fibroblast-like primary stroma was observed only with Ad5 despite the observation that none of the known receptor molecules are expressed on these cells (see, Table III). The absence of infection of human stroma using the chimeric viruses is advantageous since, in a co-culture setting, the chimeric adenovirus will not be absorbed primarily by the stroma “feeder” cells.

To test the transduction capacity of the fiber chimeric viruses, a pool of umbilical cord blood (three individuals) was used for the isolation of stem cells. CD34⁺ cells were isolated from mononuclear cell preparation using a MACS laboratory separation system (Miltenyi Biotec) using the protocol supplied by the manufacturer. Of the CD34⁺ cells, 2×10⁵ were seeded in a volume of 150 μl DMEM (no serum; Gibco, Gaithersburg, Md.) and 10 μl of chimeric adenovirus (to give a final virus particles/cell ratio of 1000) was added. The chimeric adenoviruses tested were Ad5, Ad5.Fib16, Ad5.Fib35, Ad5Fib17, Ad5.Fib51, all containing GFP as a marker. Cells were incubated for two hours in a humidified atmosphere of 10% CO₂ at 37° C. Thereafter, cells were washed once with 500 μl DMEM and re-suspended in 500 μl of StemPro-34 SF medium (Life Technologies, Grand Island, N.Y.).

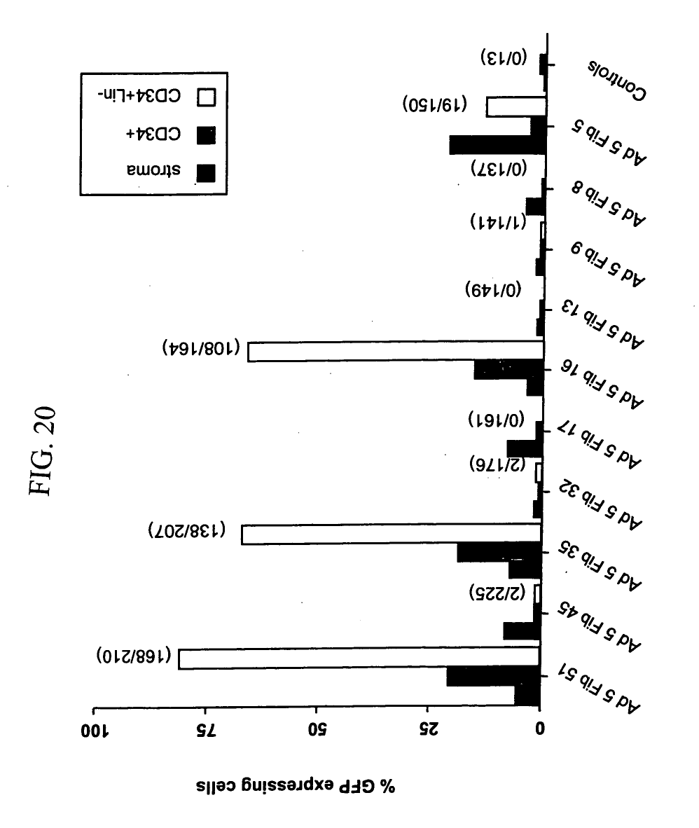

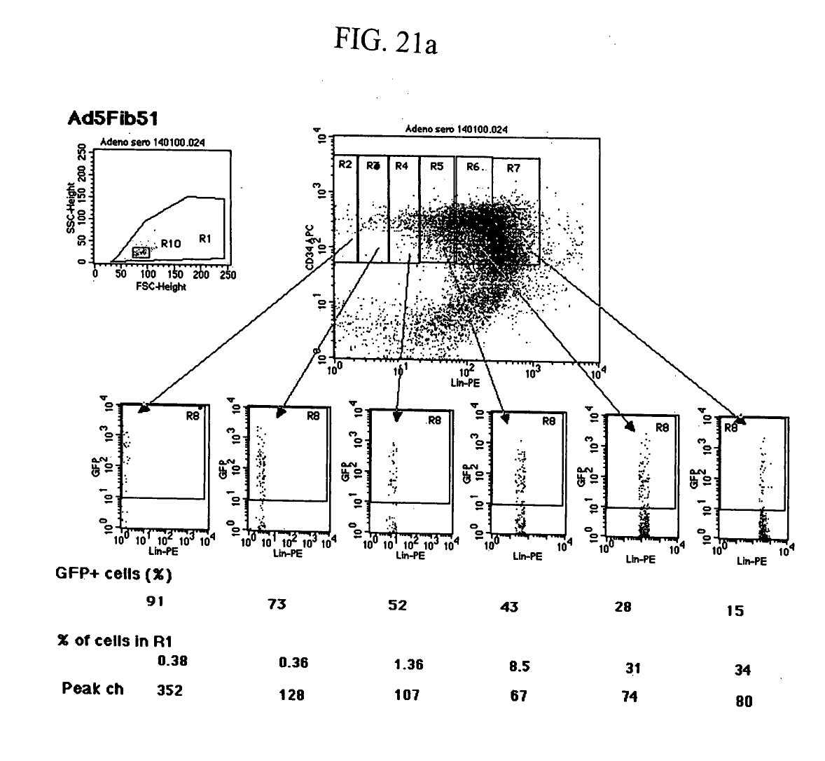

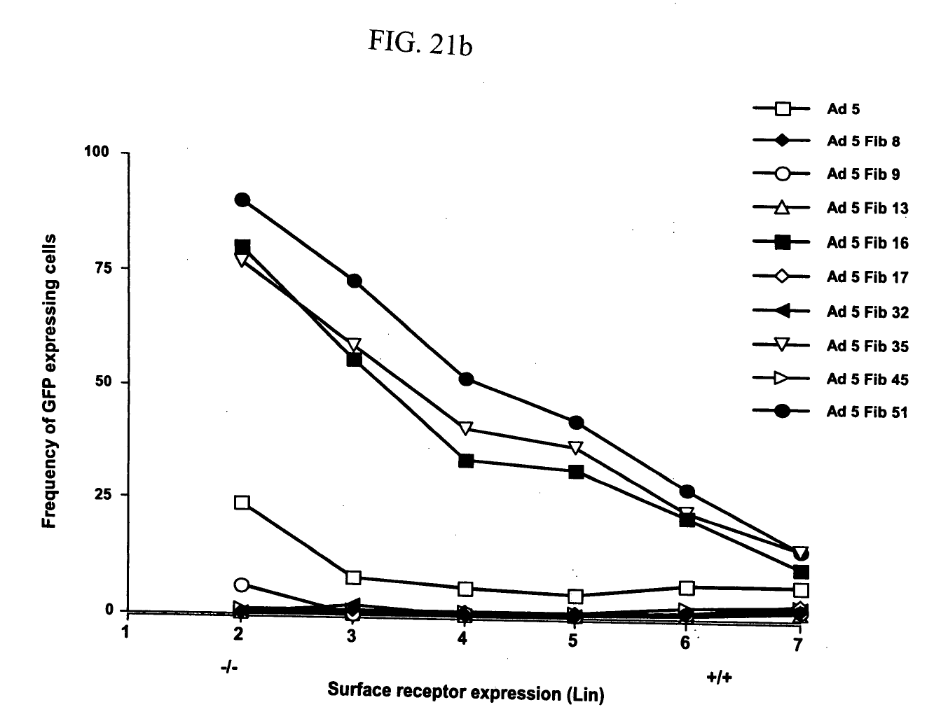

Cells were then cultured for five days in 24-well plates (Greiner, Frickenhausen, Germany) on irradiated (20 Gy) pre-established human bone marrow stroma (ref 1), in a humidified atmosphere of 10% CO₂ at 37° C. After five days, the entire cell population was collected by trypsinization with 100 μl 0.25% Trypsin-EDTA (Gibco). The number of cells before and after five days of culture was determined using a hematocytometer. The number of CD34⁺ and CD34⁺⁺ CD33, 38, 71⁻ cells in each sample was calculated from the total number of cells recovered and the frequency of the CD34⁺⁺ CD33, 38, 71⁻ cells in the whole population as determined by FACS analysis. The transduction efficiency was determined by FACS analysis while monitoring in distinct subpopulations the frequency of GFP-expressing cells, as well as the intensity of GFP per individual cell. The results of this experiment, shown in FIG. 20, demonstrates that Ad5 or the chimeric adenovirus Ad5.Fib17 does not infect CD34⁺Lin⁻ cells as witnessed by the absence of GFP expression. In contrast, with the chimeric viruses carrying the fiber molecule of serotypes 16, 51, or 35 high percentages of GFP-positive cells are scored in this cell population. Specificity for CD34⁺Lin⁻ is demonstrated since little GFP expression is observed in CD34⁺ cells that are also expressing CD33, CD38, and CD71. Sub-fractioning of the CD34⁺Lin⁻ cells (FIG. 21) showed that the percentage of cells positive for GFP declines using Ad5.Fib16, Ad5.Fib35, or Ad5.Fib51when the cells become more and more positive for the early differentiation markers CD33 (myeloid), CD71 (erythroid), and CD38 (common early differentiation marker). These results thus demonstrate the specificity of the chimeric adenoviruses Ad5.Fib 16, Ad5.Fib35, and Ad5.Fib51 for HSCs.

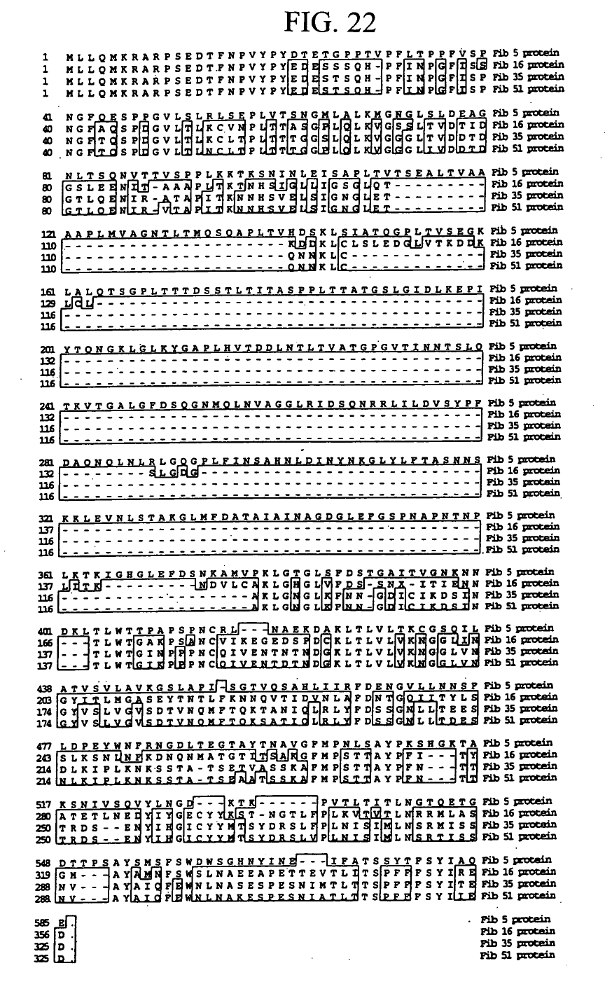

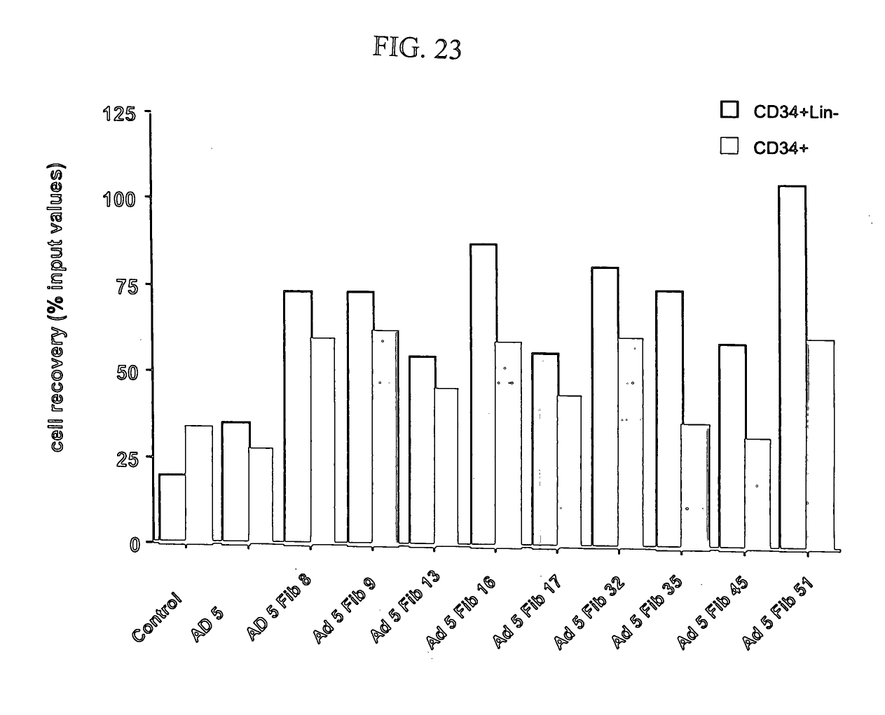

FIG. 22 shows an alignment of the Ad5 fiber with the chimeric B-group fiber proteins derived from Ad16, 35 and 51. By determining the number of cells recovered after the transduction procedure, the toxicity of adenovirus can be determined. The recovery of the amount of CD34+ cells, as well as the amount of CD34⁺Lin⁻ (FIG. 23), demonstrates that a two-hour exposure to 1000 adenovirus particles did not have an effect on the number of cells recovered.

### Example 10

An Ad5/Fiber35 Chimeric Vector with Cell Type Specificity for Dendritic Cells

Dendritic cells are antigen-presenting cells (“APC”) specialized to initiate a primary immune response and able to boost a memory type of immune response. Dependent on their stage of development, DC display different functions: immature DC are very efficient in the uptake and processing of antigens for presentation by Major Histocompatibility Complex (“MHC”) class I and class II molecules, whereas mature DC, being less effective in antigen capture and processing, perform much better at stimulating naive and memory CD4⁺ and CD8⁺ T-cells, due to the high expression of MHC molecules and co-stimulatory molecules at their cell surface. The immature DCs mature in vivo after uptake of antigen, travel to the T-cell areas in the lymphoid organs, and prime T-cell activation.

Since DCs are the cells responsible for triggering an immune response, there has been a long-standing interest in loading DCs with immunostimulatory proteins, peptides, or the genes encoding these proteins, to trigger the immune system. The applications for this strategy are in the field of cancer treatment as well as in the field of vaccination. So far, anti-cancer strategies have focused primarily on ex vivo loading of DCs with antigen (protein or peptide). These studies have revealed that this procedure resulted in induction of cytotoxic T-cell activity. The antigens used to load the cells are generally identified as being tumor specific. Some non-limiting examples of such antigens are GP 100, mage, or Mart-1 for melanoma.

Besides treatment of cancer, many other potential human diseases are currently being prevented through vaccination. In the vaccination strategy, a “crippled” pathogen is presented to the immune system via the action of the antigen-presenting cells, i.e., the immature DCs. Well-known examples of disease prevention via vaccination strategies include Hepatitis A, B, and C, influenza, rabies, yellow fever, and measles. Besides these well-known vaccination programs, research programs for treatment of malaria, ebola, river blindness, HIV and many other diseases are being developed. Many of the identified pathogens are considered too dangerous for the generation of “crippled” pathogen vaccines. This latter thus calls for the isolation and characterization of proteins of each pathogen to which a “full-blown” immune response is mounted, thus resulting in complete protection upon challenge with wild-type pathogen.

For the strategy of loading DCs with immunostimulatory proteins or peptides to become therapeutically feasible, at least two distinct criteria have to be met. First, the isolation of large numbers of DCs that can be isolated, manipulated, and re-infused into a patient, making the procedure autologous. To date, it is possible to obtain such large quantities of immature DCs from cultured peripheral blood monocytes from any given donor. Second, a vector that can transduce DCs efficiently such that the DNA encoding for an immunostimulatory protein can be delivered. The latter is extremely important since it has become clear that the time required for DCs to travel to the lymphoid organs is such that most proteins or peptides are already released from the DCs, resulting in incomplete immune priming. Because DCs are terminally differentiated and thus non-dividing cells, recombinant adenoviral vectors are being considered for delivering the DNA encoding for antigens to DCs. Ideally, this adenovirus should have a high affinity for dendritic cells, but should also not be recognized by neutralizing antibodies of the host such that in vivo transduction of DCs can be accomplished. The latter would obviate the need for ex vivo manipulations of DCs but would result in a medical procedure identical to the vaccination programs that are currently in place, i.e., predominantly intramuscular or subcutaneous injection. Thus, DC transduced by adenoviral vectors encoding an immunogenic protein may be ideally suited to serve as natural adjuvants for immunotherapy and vaccination.

From the described examples, it can be concluded that efficient gene delivery to DCs is a major interest in the field of gene therapy. Therefore, alteration of the Ad5 host cell range to be able to target DCs in vitro as well as in vivo is a major interest of the invention. To identify a chimeric adenovirus with preferred infection characteristics for human DCs, a library of Ad5-based viruses carrying the fiber molecule from alternative serotypes (serotypes 8, 9, 13, 16, 17, 32, 35, 45, 40-L, 51) was generated. Ad5 was included as a reference.

The susceptibility of human monocyte-derived immature and mature DC to recombinant chimeric adenoviruses expressing different fibers was evaluated.

Human PBMC from healthy donors were isolated through Ficoll-Hypaque density centrifugation. Monocytes were isolated from PBMC by enrichment for CD14⁺ cells using staining with FITC-labelled anti-human CD 14 monoclonal antibody (Becton Dickinson), anti-FITC microbeads and MACS separation columns (Miltenyi Biotec).

This procedure usually results in a population of cells that are <90% CD14⁺ as analyzed by FACS. Cells were placed in culture using RPMI-1640 medium (Gibco) containing 10% Fetal Bovine Serum (“FBS”) (Gibco), 200 ng/ml rhu GM-CSF (R&D/ITK diagnostics), 100 ng/ml rhu IL-4 (R&D/ITK diagnostics) and cultured for seven days with feeding of the cultures with fresh medium containing cytokines on alternate days. After seven days, the immature DC resulting from this procedure express a phenotype CD83⁻, CD14^{low} or CD14⁻, HLA-DR⁺, as was demonstrated by FACS analysis. Immature DCs are matured by culturing the cells in a medium containing 100 ng/ml TNF-α for three days, after which, they expressed CD83 on their cell surface.

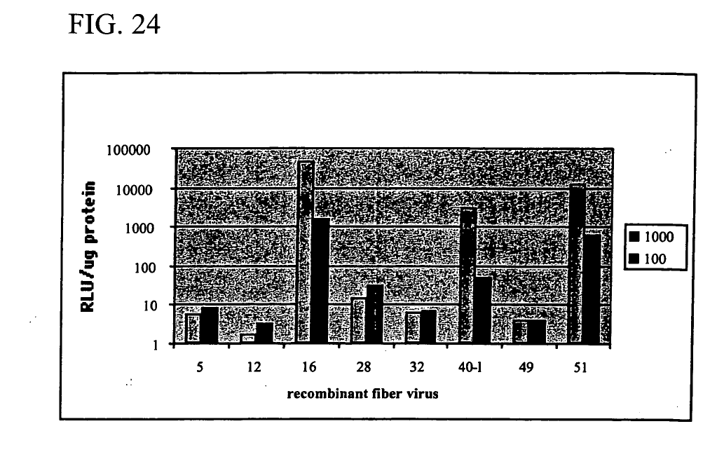

In a pilot experiment, 5×10⁵ immature DCs were seeded in wells of 24-well plates and exposed for 24 hours to 100 and 1000 virus particles per cell of each fiber-recombinant virus. Virus tested was Ad5, and the fiber chimeric viruses based on Ad5: Ad5.Fib12, Ad5.Fib16, Ad5.Fib28, Ad5.Fib32, Ad5.Fib40-L (long fiber of serotype 40), Ad5.Fib49, and Ad5.Fib51 (where Fibxx stands for the serotype from which the fiber molecule is derived). These viruses are derived from subgroup C, A, B, D, D, F, D, and B, respectively. After 24 hours, cells were lysed (1% Triton X-100/PBS) and luciferase activity was determined using a protocol supplied by the manufacturer (Promega, Madison, Wis., U.S.A.). The results of this experiment, shown in FIG. 24, demonstrate that Ad5 poorly infects immature DCs as witnessed by the low level of transgene expression. In contrast, Ad5.Fib16 and Ad5.Fib51 (both a B-group fiber chimeric virus) and also Ad5.Fib40-L (Subgroup F) show efficient infection of immature DCs based on luciferase transgene expression.

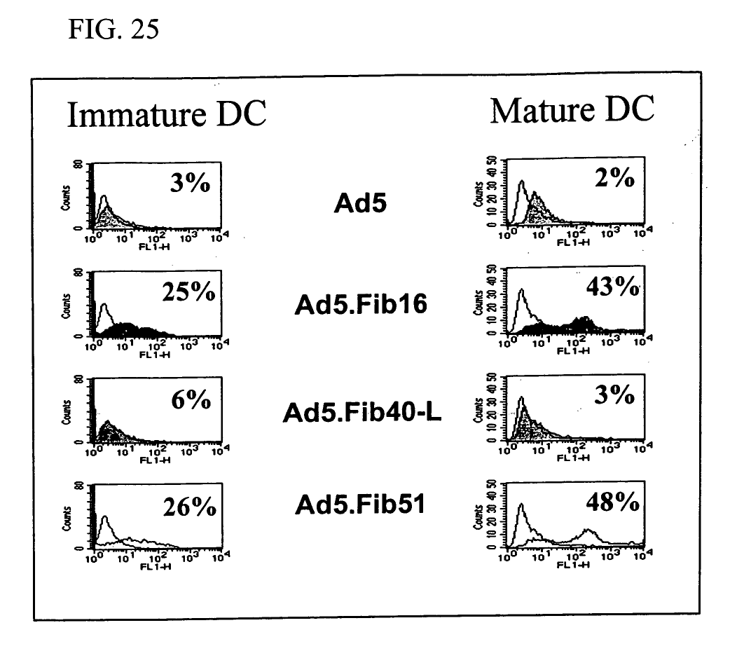

In a second experiment, 5×10⁵ immature and mature DC were infected with 10,000 virus particles per cell of Ad5, Ad5.Fib16, Ad5.Fib40-L, and Ad5.Fib51, all carrying the LacZ gene as a marker. LacZ expression was monitored by flow cytometric analysis using a CM-FDG kit system and the instructions supplied by the manufacturer (Molecular Probes, Leiden, NL). The results of this experiment, shown in FIG. 25, correlate with the previous experiment in that Ad5.Fib16 and Ad5.Fib51 are superior to Ad5 in transducing mature and immature human DCs. Also, this experiment shows that Ad5.Fib40-L is not as good as Ad5.Fib16 and Ad5.Fib51, but is better than Ad5.

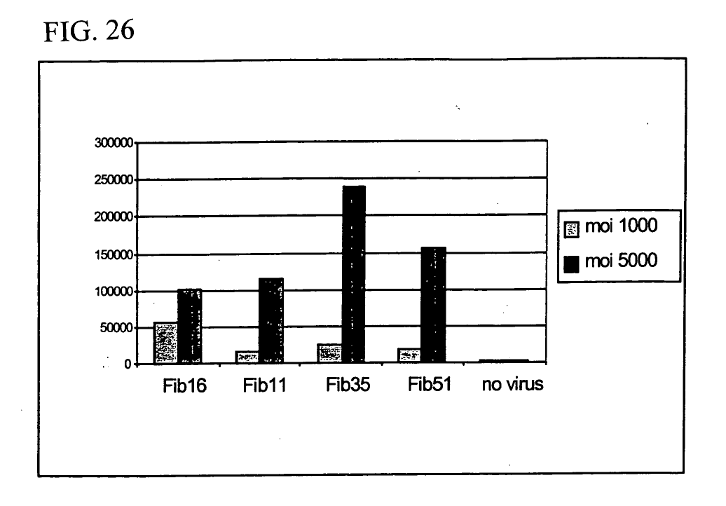

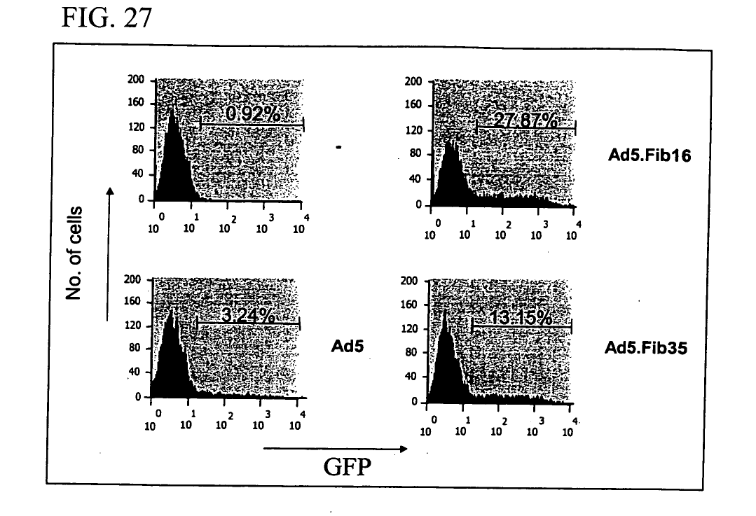

Based on these results, we tested other chimeric adenoviruses containing fibers of B group viruses, for example, Ad5.Fib11 and Ad5.Fib35, for their capacity to infect DCs. We focused on immature DCs, since these are the cells that process an expressed transgene product into MHC class I and II presentable peptides. Immature DCs were seeded at a cell density of 5×10⁵ cells/well in 24-well plates (Costar) and infected with 1,000 and 5,000 virus particles per cell after which the cells were cultured for 48 hours under conditions for immature DCs prior to cell lysis and Luciferase activity measurements. The results of this experiment, shown in FIG. 26, demonstrate that Ad5-based chimeric adenoviruses containing fibers of group-B viruses efficiently infect immature DCs. In a fourth experiment, we again infected immature DCs identically as described in the former experiments but this time Ad5, Ad5.Fib16, and Ad5.Fib35 were used carrying GFP as a marker gene. The results on GFP expression measured with a flow cytometer 48 hours after virus exposure is shown in FIG. 27 and correlates with the data obtained so far. Thus, the results so far are consistent in that Ad5-based vectors carrying a fiber from an alternative adenovirus derived from subgroup B predominantly fiber of 35, 51, 16, and 11 are superior to Ad5 for transducing human DCs.

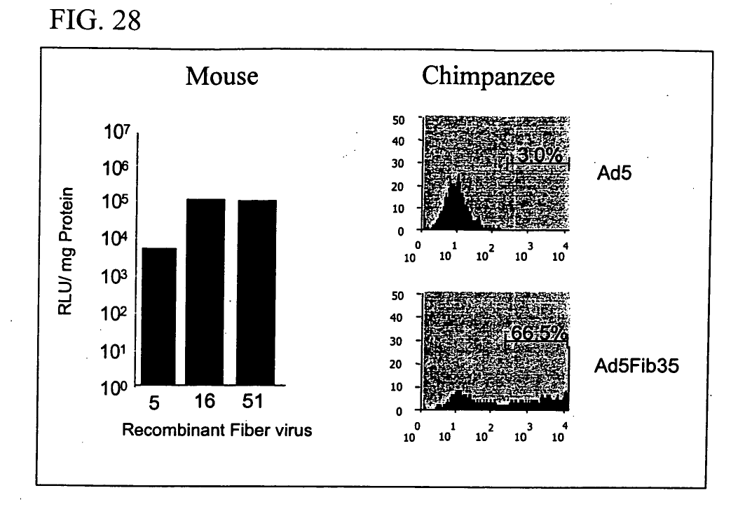

The adenoviruses disclosed herein are also very suitable for vaccinating animals. To illustrate this, we tested DCs derived from mice and chimpanzees to identify whether these viruses could be used in these animal models; the latter, in particular, since the receptor for human adenovirus derived from subgroup B is unknown to date and, therefore, it is unknown whether this protein is conserved among species. For both species, immature DCs were seeded at a density of 10⁵ cells per well of 24-well plates. Cells were subsequently exposed for 48 hours to 1000 virus particles per cell of Ad5, Ad5Fib16, and Ad5.Fib51in case of mouse DC and Ad5, and Ad.Fib35 in case of chimpanzee DCs (see, FIG. 28). The mouse experiment was performed with viruses carrying luciferase as a marker, and demonstrated approximately 10- to 50-fold increased luciferase activity as compared to Ad5.

The chimpanzee DCs were infected with the GFP viruses, and were analyzed using a flow cytometer. These results (also shown in FIG. 28) demonstrate that Ad5 (3%) transduces chimpanzee DCs very poorly as compared to Ad5.Fib35 (66.5%).

### Example 11

Construction of a Plasmid-Based Vector System to Generate Ad11-Based Recombinant Viruses

The results of the neutralization experiments described in Example 5 show that Ad11, like Ad35, was also not neutralized in the vast majority of human serum samples. Therefore, recombinant adenoviruses based on Ad11 are preferred above the commonly used Ad2 and Ad5-based vectors as vectors for gene therapy treatment and vaccination. Both Ad35 and Ad11 are B-group viruses and are classified as viruses belonging to DNA homology cluster 2 (Wadell, 1984). Therefore, the genomes of Ad35 and Ad11 are very similar.

To generate a plasmid-based system for the production of Ad11-based recombinant viruses, the adapter plasmid pAdApt35IP1 generated in Example 7 is modified as follows. Construct pAdApt35IP1 is digested with AvrII and then partially with PacI. The digestion mixture is separated on gel, and the 4.4 kb fragment containing the expression cassette and the vector backbone is isolated using the GENECLEAN kit (BIO 101, Inc.). Then, a PCR amplification is performed on wtAd11 DNA using the primers 35F1 and 35R2 (see, Example 7) using Pwo DNA polymerase according to the manufacturer's instructions. The obtained PCR fragment of 0.5 kb is purified using the PCR purification kit (LTI) and ligated to the previously prepared fragment of pAdApt35IP1. This gives construct pAdAptll-351P1, in which the 5′ adenovirus fragment is exchanged for the corresponding sequence of Ad11. Next, pAdApt11-351P1 is digested with BglII and partially with PacI. The obtained fragments are separated on gel, and the 3.6 kb fragment containing the vector sequences, the 5′ adenovirus fragment, and the expression cassette is purified from gel as previously described. Next, a PCR fragment is generated using primers 35F3 and 35R4 (see, Example 7) on wtAd11 DNA. Amplification is done as above and the obtained 1.3 kb fragment is purified and digested with BglII and PacI. The isolated fragments are then ligated to give construct pAdApt11IP1. This adapter plasmid now contains Ad11 sequences instead of Ad35 sequences. Correct amplification of PCR-amplified Ad11 sequences is verified by comparison of the sequence in this clone with the corresponding sequence of Ad11 DNA. The latter is obtained by direct sequencing on Ad11 DNA using the indicated PCR primers. The large cosmid clone containing the Ad11 backbone is generated as follows. First, a PCR fragment is amplified on Ad11 DNA using the primers 35F5 and 35R6 with Pwo DNA polymerase as described in Example 7 for Ad35 DNA. The PCR fragment is then purified using the PCR purification kit (LTI) and digested with NotI and NdeI. The resulting 3.1 kb fragment is isolated from gel using the GENECLEAN kit (Bio 101, Inc.). A second PCR fragment is then generated on Ad11 DNA using the primers 35F7 and 35R8 (see, Example 7) with Pwo DNA polymerase according to the manufacturer's instructions and purified using the PCR purification kit (LTI). This amplified fragment is also digested with NdeI and NotI and the resulting 1.6 kb fragment is purified from gel as previously described. The two digested PCR fragments are then ligated together with cosmid vector pWE15 previously digested with NotI and dephosphorylated using Tsap enzyme (LTI) according to the manufacturer's instructions. One clone is selected that has one copy of both fragments inserted. Correct clones are selected by analytical NotI digestion that gives a fragment of 4.7 kb. Confirmation is obtained by a PCR reaction using primers 35F5 and 35R8 that gives a fragment of the same size. The correct clone is then linearized with NdeI and isolated from gel. Next, wtAd11 DNA is digested with NdeI and the large 27 kb fragment is isolated from low-melting point agarose gel using agarase enzyme (Roche) according to the manufacturer's instructions. Both fragments are then ligated and packaged using λ phage packaging extracts (Stratagene) according to the manufacturer's protocol. After infection into STBL-2 cells (LTI), colonies are grown on plates, and analyzed for the presence of the complete insert. The functionality of selected clones is then tested by co-transfection on PER.C6. Hereto, the DNA is digested with NotI and 6 μgr is co-transfected with 2 μgr of a PCR fragment generated on Ad11 DNA with primers 35F1 and 35R4 (see, Example 7). Correct clones give CPE within one week following transfection. The correct clone is designated pWE.Ad11.pIX-rITR.

Using the previously described procedure, a plasmid-based system consisting of an adapter plasmid suitable for insertion of foreign genes and a large helper fragment containing the viral backbone is generated. Recombinant Ad11-based viruses are made using the methods described herein for Ad35-based recombinant viruses.

### Example 12

Neutralization of Adenoviruses in Samples Derived from Patients

In the neutralization experiments described in Examples 1 and 5, all samples were derived from healthy volunteers. Since one of the applications of non-neutralized vectors is in the field of gene therapy, it is interesting to investigate whether Ad35 is also neutralized with a low frequency and with low titers in groups of patients that are candidates for treatment with gene therapy.

Cardio-Vascular Disease Patients

Twenty-six paired serum and pericardial fluid (PF) samples were obtained from patients with heart failure. These were tested against Ad5 and Ad35 using the neutralization assay described in Example 1. The results confirmed the previous data with samples from healthy volunteers. 70% of the serum samples contained NA to Ad5 and 4% to Ad35. In the pericardial fluid samples, the titers were lower resulting in a total of 40% with NA to Ad5 and none to Ad35. There was a good correlation between NA in PF and serum, i.e., there were no positive PF samples without NA in the paired serum sample. These results show that non-neutralized vectors based on Ad35 are preferred over Ad5 vectors for treatment of cardio-vascular diseases. As is true for all forms of non-neutralized vectors in this application, the vector may be based on the genome of the non-neutralized serotype or may be based on Ad5 (or another serotype) through displaying at least the major capsid proteins (hexon, penton and optionally fiber) of the non-neutralized serotype.

Rheumatoid Arthritis Patients

The molecular determinant underlying arthritis is presently not known, but both T-cell dysfunction and imbalanced growth factor production in joints is known to cause inflammation and hyperplasia of synovial tissue. The synoviocytes start to proliferate and invade the cartilage and bone that leads to destruction of these tissues. Current treatment starts (when in an early stage) with administration of anti-inflammatory drugs (anti-TNF, IL1-RA, IL-10) and/or conventional drugs (e.g., MTX, sulfasalazine). In late stage RA, synovectomy is performed which is based on surgery, radiation, or chemical intervention. An alternative or additional option is treatment via gene therapy where an adenoviral vector is delivered directly into the joints of patients and expresses an anti-inflammatory drug or a suicide gene. Previous studies performed in rhesus monkeys suffering from collagen-induced arthritis have shown that Ad5-based vectors carrying a marker gene can transduce synoviocytes. Whether, in the human situation, adenoviral delivery is hampered by the presence of NA is not known. To investigate the presence of NA in the synovial fluid (“SF”) of RA patients, SF samples were obtained from a panel of 53 randomly selected patients suffering from RA. These were tested against several wt adenoviruses using the neutralization assay described in Example 1. Results of this screen are presented in Table III. Adenovirus type 5 was found to be neutralized in 72% of the SF samples. Most of these samples contain high titers of NA since the highest dilution of the SF sample that was tested (64×), neutralized Ad5 viruses. This means that adenoviral vector delivery to the synoviocytes in the joints of RA patients will be very inefficient. Moreover, since the titers in the SF are so high, it is doubtful whether lavage of the joints prior to vector injection will remove enough of the NA. Of the other serotypes that were tested, Ad35 was shown to be neutralized in only 4% of the samples. Therefore, these data confirm the results obtained in serum samples from healthy patients and show that, for treatment of RA, Ad35-based vectors or chimeric vectors displaying at least some of the capsid proteins from Ad35 are preferred vectors.

### Example 13

Modifications in the Backbone of Ad35-Based Viruses

Generation of pBr/Ad35.Pac-rITR and pBr/Ad35.PRn

Example 4 describes the generation of the Ad35 subclone pBr/Ad35.Eco13.3. This clone contains Ad35 sequences from bp 21943 to the end of the right ITR cloned into the EcoRI and EcoRV sites of pBr322. To extend these sequences to the PacI site located at bp 18137 in Ad35, pBr/Ad35.Eco13.3 (see Example 4) was digested with AatII and SnaBI and the large vector—containing fragment was isolated from gel using the QIAEX II gel extraction kit (Qiagen). Ad35 wt DNA was digested with PacI and SnaBI and the 4.6 kb fragment was isolated as above. This fragment was then ligated to a double-stranded (“ds”) linker containing a PacI and an AatII overhang. This linker was obtained after annealing the following oligonucleotides: A-P1: 5′-CTG GTG GTT AAT-3′ (SEQ ID NO:61) and A-P2: 5′-TAA CCA CCA GAC GT-3′ (SEQ ID NO:62)

The ligation mix containing the double-stranded linker and the PacI-SnaBI Ad35 fragment was separated from unligated linker on a LMP gel. The 4.6 kb band was cut out of the gel, molten at 65° C. and then ligated to the purified pBr/Ad35.Eco13.3 vector fragment digested with AatII and SnaBI. Ligations were transformed into electrocompetent DH10B cells (Life Technologies Inc.). The resulting clone, pBr/Ad35.Pac-rITR, contained Ad35 sequences from the PacI site at bp 18137 up to the right ITR.

Next, a unique restriction site was introduced at the 3′ end of the right ITR to be able to free the ITR from vector sequences. Hereto, a PCR fragment was used that covers Ad35 sequences from the NdeI site at bp 33165 to the right ITR having the restriction sites Swal, NotI and EcoRI attached to the rITR. The PCR fragment was generated using primers 35F7 and 35R8 (described in Example 7). After purification, the PCR fragment was cloned into the AT cloning vector (Invitrogen) and sequenced to verify correct amplification. The correct amplified clone was then digested with EcoRI, blunted with Klenow enzyme and subsequently digested with NdeI and the PCR fragment was isolated. In parallel, the NdeI in the pBr vector in pBr/Ad35.Pac-rITR was removed as follows: A pBr322 vector from which the NdeI site was removed by digestion with NdeI, Klenow treatment and religation, was digested with AatII and NheI. The vector fragment was isolated in LMP gel and ligated to the 16.7 kb Ad35 AatII-NheI fragment from pBr/Ad35.Pac-rITR that was also isolated in an LMP gel. This generated pBr/Ad35.Pac-rITR.ΔNdeI. Next, pBr/Ad35.Pac-rITR.ΔNdeI was digested with NheI, the ends were filled in using Klenow enzyme, and the DNA was then digested with NdeI. The large fragment containing the vector and Ad35 sequences was isolated. Ligation of this vector fragment and the PCR fragment resulted in pBr/Ad35.PRn. In this clone, specific sequences coding for fiber, E2A, E3, E4 or hexon can be manipulated. In addition, promoter sequences that drive, for instance, the E4 proteins or the E2 can be mutated or deleted and exchanged for heterologous promoters.

Generation of Ad35-Based Viruses with Fiber Proteins from Different Serotypes.

Adenoviruses infect human cells with different efficiencies. Infection is accomplished by a two-step process involving both the fiber proteins that mediate binding of the virus to specific receptors on the cells, and the penton proteins that mediate internalization by interaction of, for example, the RGD sequence to integrins present on the cell surface. For subgroup B viruses, of which Ad35 is a member, the cellular receptor for the fiber protein is not known. Striking differences exist in infection efficiency of human cells of subgroup B viruses compared to subgroup C viruses like Ad5 (see, International Patent Application WO 00/03029 and European Patent Application EP 99200624.7). Even within one subgroup infection, efficiencies of certain human cells may differ between various serotypes. For example, the fiber of Ad16, when present on an Ad5-based recombinant virus infects primary endothelial cells, smooth muscle cells and synoviocytes of human and rhesus monkey origin better than Ad5-chimeric viruses carrying the fiber of Ad35 or Ad51. Thus, to obtain high infection efficiencies of Ad35-based viruses, it may be necessary to change the fiber protein for a fiber protein of a different serotype. The technology for such fiber chimeras is described for Ad5-based viruses in Example 3, and is below exemplified for Ad35 viruses.

First, most fiber sequences are deleted from the Ad35 backbone in construct pBr/Ad35.PRn as follows:

The left-flanking sequences and part of the fiber protein in Ad35 ranging from bp 30225 upstream of a unique MluI site up to bp 30872 (numbers according to wt Ad35 sequence as disclosed in SEQ ID NO:82) in the tail of fiber are amplified using primers DF35-1: 5′-CAC TCA CCA CCT CCA ATT CC-3′ (SEQ ID NO:63) and DF35-2: 5′-CGG GAT CCC GTA CGG GTA GAC AGG GTT GAA GG-3′ (SEQ ID NO:64). This PCR amplification introduces a unique BsiWI site in the tail of the fiber gene. The right-flanking sequences ranging from the end of the fiber protein at bp 31798 to bp 33199 (numbering according to wtAd35 sequence (SEQ ID NO:82)), 3′ from the unique NdeI site is amplified using primers DF35-3: 5′-CGG GAT CCG CTA GCT GAA ATA AAG TTT AAG TGT TTT TAT TTA AAA TCA C-3′ (SEQ ID NO:65) and DF35-4: 5′-CCA GTT GCA TTG CTT GGT TGG-3′ (SEQ ID NO:66). This PCR introduces a unique NheI site in the place of the fiber sequences. PCR amplification is done with Pwo DNA polymerase (Roche) according to the manufacturer's instructions. After amplification, the PCR products are purified using a PCR purification kit and the fragments are digested with BamHI and ligated together. The 2 kb ligated fragments are purified from gel, and cloned in the PCR Script Amp vector (Stratagene). Correct amplification is checked by sequencing. The PCR fragment is then excised as a MluI/NdeI fragment and cloned in pBr/Ad35.PRn digested with the same enzymes. This generates pBr/Ad35.PRΔfib, a shuttle vector suitable to introduce fiber sequences of alternative serotypes. This strategy is analogous to the fiber-modification strategy for Ad5-based viruses as disclosed in International Patent Application WO00/03029. Primers that are listed in Table I of that application were used to amplify fiber sequences of various subgroups of adenovirus. For amplification of fibers that are cloned in the pBr/Ad35.PRΔfib, the same (degenerate) primer sequences can be used, however, the NdeI site in the forward primers (tail oligonucleotides A to E) should be changed to a BsiWI site and the NsiI site in the reverse oligo (knob oligonucleotide 1 to 8) should be changed in a NheI site. Thus, fiber 16 sequences are amplified using the following degenerate primers: 5′-CCK GTS TAC CCG TAC GAA GAT GAA AGC-3′ (SEQ ID NO:67) (where K can be a T or G and S can be a C or G, as both are degenerate oligo nucleotides) and 5′-CCG GCT AGC TCA GTC ATC TTC TCT GAT ATA-3′ (SEQ ID NO:68). Amplified sequences are then digested with BsiWI and NheI and cloned into pBr/Ad35.PRΔfib digested with the same enzymes to generate pBr/Ad35.PRfib16. The latter construct is then digested with PacI and SwaI and the insert is isolated from gel. The PacI/SwaI Ad35 fragment with modified fiber is then cloned into the corresponding sites of pWE/Ad35.pIX-rITR to give pWE/Ad35.pIX-rITR.fib16. This cosmid backbone can then be used with an Ad35-based adapter plasmid to generate Ad35-recombinant viruses that display the fiber of Ad16. Other fiber sequences can be amplified with (degenerate) primers as mentioned above. If one of the fiber sequences turns out to have an internal BsiWI or NheI site, the PCR fragment has to be digested partially with that enzyme.

Generation of Ad35-Based Viruses with Inducible, E1 Independent, E4 Expression

The adenovirus E4 promoter is activated by expression of E1 proteins. It is unknown whether the Ad5 E1 proteins are capable of mediating activation of the Ad35 E4 promoter. Therefore, to enable production of Ad35-recombinant viruses on PER.C6 cells, it may be advantageous to make E4 expression independent of E1. This can be achieved by replacing the Ad35-E4 promoter by heterologous promoter sequences like, but not limited to, the 7xTetO promoter.

Recombinant E1-deleted Ad5-based vectors are shown to have residual expression of viral genes from the vector backbone in target cells, despite the absence of E1 expression. Viral gene expression increases the toxicity and may trigger a host immune response to the infected cell. For most applications of adenoviral vectors in the field of gene therapy and vaccination, it is desired to reduce or diminish the expression of viral genes from the backbone. One way to achieve this is to delete all, or as much as possible, sequences from the viral backbone. By deleting E2A, E2B or E4 genes and/or the late gene functions, one has to complement for these functions during production. This complementation can either be by means of a helper virus or through stable addition of these functions, with or without inducible transcription regulation, to the producer cell. Methods to achieve this have been described for Ad5 and are known in the art. One specific method is replacement of the E4 promoter by promoter sequences that are not active in the target cells. E4 proteins play a role in, for example, replication of adenoviruses through activation of the E2 promoter and in late gene expression through regulation of splicing and nuclear export of late gene transcripts. In addition, at least some of the E4 proteins are toxic to cells. Therefore, reduction or elimination of E4 expression in target cells will further improve Ad35-based vectors. One way to achieve this is to replace the E4 promoter by a heterologous promoter that is inactive in the target cells. An example of a heterologous promoter/activator system that is inactive in target cells is the tetracycline-inducible TetO system (Gossen and Bujard, 1992). Other prokaryotic or synthetic promoter/activator systems may be used. In this example, the E4 promoter in the backbone of the viral vector is replaced by a DNA fragment containing seven repeats of the tetracycline responsive element from the tet operon (7xTetO). A strong transactivator for this promoter is a fusion protein containing the DNA binding domain of the tet repressor and the activation domain of VP16 (Tet transactivator protein, Tta). Strong E4 expression, independent of E1 expression, can be accomplished in PER.C6 cells expressing Tta. Tta-expressing PER.C6 cells have been generated and described (see, Example 15). Ad5 derived E1-deleted viruses with E4 under control of 7xTetO can be generated and propagated on these cells. Following infection in cells of human or animal origin (that do not express the Tta transactivator), E4 expression was found to be greatly diminished compared to E1-deleted viruses with the normal E4 promoter.

What follows is the construction of pWE/Ad35.pIX-rITR.TetO-E4, a cosmid helper vector to produce viruses with the E4 promoter replacement.

First, a fragment was generated by PCR amplification on pBr/Ad35.PRn DNA using the following primers: 355ITR: 5′-GAT CCG GAG CTC ACA ACG TCA TTT TCC CAC G-3′ (SEQ ID NO:69) and 353ITR: 5′-CGG AAT TCG CGG CCG CAT TTA AAT C-3′ (SEQ ID NO:70). This fragment contains sequences between bp 34656 (numbering according to wtAd35) and the NotI site 3′ of the right ITR in pBr/Ad35.PRn and introduces an SstI site 5′ of the right ITR sequence.

A second PCR fragment was generated on pBr/Ad35.PRn DNA using primers: 35DE4: 5′-CCC AAG CTT GCT TGT GTA TAT ATA TTG TGG-3′ (SEQ ID NO:71) and 35F7 (see Example 7). This PCR amplifies Ad35 sequences between bp 33098 and 34500 (numbering according to wtAd35) and introduces a HindIII site upstream of the E4 Tata-box. With these two PCR reactions, the right- and left-flanking sequences of the E4 promoter are amplified. For amplification, Pwo DNA polymerase was used according to the manufacturer's instructions.

A third fragment containing the 7xTetO promoter was isolated from construct pAAO-E-TATA-7xTetO by digestion with SstI and HindIII. The generation of pAAO-E-TATA-7xTetO is described below. The first PCR fragment (355/353) was then digested with SstI and NotI and ligated to the 7xTetO fragment. The ligation mixture was then digested with HindIII and NotI and the 0.5 kb fragment is isolated from gel. The second PCR fragment (35DE4/35F7) was digested with NdeI and HindIII and gel purified. These two fragments are then ligated into pBr/Ad35.PRn digested with NdeI and NotI to give pBr/Ad35.PR.TetOE4. The modification of the E4 promoter is then transferred to the Ad35 helper cosmid clone by exchanging the PacI/SwaI fragment of the latter with the one from pBr/Ad35.PR.TetOE4 to give pWE/Ad35.pIX-rITR.TetOE4.

pAAO-E-TATA.7xTetO was generated as follows. Two oligonucleotides were synthesized: TATAplus: 5′-AGC TTT CTT ATA AAT TTT CAG TGT TAG ACT AGT AAA TTG CTT AAG-3′ (SEQ ID NO:72) and TATAmin: 5′-AGC TCT TAA GCA ATT TAC TAG TCT AAC ACT GAA AAT TTA TAA GAA-3′ (SEQ ID NO:73).

(The underlined sequences form a modified TATA box.) The oligonucleotides were annealed to yield a double-stranded DNA fragment with 5′ overhangs that are compatible with HindIII-digested DNA. The product of the annealing reaction was ligated into HindIII-digested pGL3-Enhancer Vector (Promega) to yield pAAO-E-TATA. The clone that had the HindIIIsite at the 5′ end of the insert restored was selected for further cloning.

Next, the heptamerized tet-operator sequence was amplified from the plasmid pUHC-13-3 (Gossen and Bujard, 1992) in a PCR reaction using the Expand PCR system (Roche) according to the manufacturer's protocol. The following primers were used: Tet3: 5′-CCG GAG CTC CAT GGC CTA ACT CGA GTT TAC CAC TCC C-3′ (SEQ ID NO:74) and Tet5: 5′-CCC AAG CTT AGC TCG ACT TTC ACT TTT CTC-3′ (SEQ ID NO:75). The amplified fragment was digested with SstI and HindIII (these sites are present in tet3 and tet5, respectively) and cloned into SstI/HindIII-digested pAAO-E-TATA giving rise to pAAO-E-TATA-7xtetO.

To test the functionality of the generated pWE/Ad35.pIX-rITR.TetOE4 cosmid clone, the DNA was digested with NotI. The left end of wtAd35 DNA was then amplified using primers 35F1 and 35R4 (see, Example 7). Following amplification, the PCR mixture was purified and digested with SalI to remove intact viral DNA. Then 4 gr of both the digested pWE/Ad35.pIX-rITR.TetOE4 and the PCR fragment was co-transfected into PER.C6-tTA cells that were seeded in T25 flasks the day before. Transfected cells were transferred to T80 flasks after two days and another two days later CPE was obtained, showing that the cosmid backbone is functional.

### Example 14

Generation of Cell Lines Capable of Complementing E1-Deleted Ad35 Viruses

Generation of pIG135 and pIG270

Construct pIG.E1A.E1B contains E1 region sequences of Ad5 corresponding to nucleotides 459 to 3510 of the wt Ad5 sequence (Genbank accession number M72360) operatively linked to the human phosphoglycerate kinase promoter (“PGK”) and the Hepatitis B Virus polyA sequences. The generation of this construct is described in International Patent Application No. WO97/00326. The E1 sequences of Ad5 were replaced by corresponding sequences of Ad35 as follows. pRSV.Ad35-E1 (described in Example 8) was digested with EcoRI and Sse83871 and the 3 kb fragment corresponding to the Ad35 E1 sequences was isolated from gel. Construct pIG.E1A.E1B was digested with Sse8387I completely and partially with EcoRI. The 4.2 kb fragment corresponding to vector sequences without the Ad5 E1 region but retaining the PGK promoter were separated from other fragments on LMP agarose gel and the correct band was excised from gel. Both obtained fragments were ligated resulting in pIG.Ad35-E1.

This vector was further modified to remove the LacZ sequences present in the pUC119 vector backbone. Hereto, the vector was digested with BsaAI and BstXI and the large fragment was isolated from gel. A double-stranded oligo was prepared by annealing the following two oligos: BB1: 5′-GTG CCT AGG CCA CGG GG-3′ (SEQ ID NO:76) and BB2: 5′-GTG GCC TAG GCA C-3′ (SEQ ID NO:77).

Ligation of the oligo and the vector fragment resulted in construct pIG135. Correct insertion of the oligo restores the BsaAI and BstXI sites and introduces a unique AvrII site. Next, we introduced a unique site at the 3′ end of the Ad35-E1 expression cassette in pIG135. Hereto, the construct was digested with SapI and the 3′ protruding ends were made blunt by treatment with T4 DNA polymerase. The thus treated linear plasmid was further digested with BsrGI and the large vector-containing fragment was isolated from gel. To restore the 3′ end of the HBVpolyA sequence and to introduce a unique site, a PCR fragment was generated using the following primers: 270F: 5′-CAC CTC TGC CTA ATC ATC TC-3′ (SEQ ID NO:78) and 270R: 5′-GCT CTA GAA ATT CCA CTG CCT TCC ACC-3′ (SEQ ID NO:79). The PCR was performed on pIG.Ad35.E1 DNA using Pwo polymerase (Roche) according to the manufacturer's instructions. The obtained PCR product was digested with BsrGI and dephosphorylated using Tsap enzyme (LTI), the latter to prevent insert dimerization on the BsrGI site. The PCR fragment and the vector fragment were ligated to yield construct pIG270.

Ad35 E1 Sequences are Capable of Transforming Rat Primary Cells

New born WAG/RIJ rats were sacrificed at one week of gestation and kidneys were isolated. After careful removal of the capsule, kidneys were disintegrated into a single cell suspension by multiple rounds of incubation in trypsin/EDTA (LTI) at 37° C. and collection of floating cells in cold PBS containing 1% FBS. When most of the kidney was trypsinized, all cells were re-suspended in DMEM supplemented with 10% FBS and filtered through a sterile cheesecloth. Baby Rat Kidney (BRK) cells obtained from one kidney and were plated in five dishes (Greiner, 6 cm). When a confluency of 70-80% was reached, the cells were transfected with 1 or 5 μgr DNA/dish using the CaPO₄ precipitation kit (LTI) according to the manufacturer's instructions. The following constructs were used in separate transfections: pIG.E1A.E1B (expressing the Ad5-E1 region), pRSV.Ad35-E1, pIG.Ad35-E1 and pIG270 (the latter expressing the Ad35-E1). Cells were incubated at 37° C., 5% CO₂ until foci of transformed cells appeared. Table IV shows the number of foci that resulted from several transfection experiments using circular or linear DNA. As expected, the Ad5-E1 region efficiently transformed BRK cells. Foci also appeared in the Ad35-E1-transfected cell layer although with lower efficiency. The Ad35 transformed foci appeared at a later time point: ˜2 weeks post-transfection compared with 7 to 10 days for Ad5-E1. These experiments clearly show that the E1 genes of the B group virus Ad35 are capable of transforming primary rodent cells. This proves the functionality of the Ad35-E1 expression constructs and confirms earlier findings of the transforming capacity of the B-group viruses Ad3 and Ad7 (Dijkema, 1979). To test whether the cells in the foci were really transformed, a few foci were picked and expanded. From the seven picked foci; at least five turned out to grow as established cell lines.

Generation of New Packaging Cells Derived from Primary Human Amniocytes

Amniotic fluid obtained after amniocentesis was centrifuged and cells were re-suspended in AmnioMax medium (LTI) and cultured in tissue culture flasks at 37° C. and 10% CO₂. When cells were growing nicely (approximately one cell division/24 hours), the medium was replaced with a 1:1 mixture of AmnioMax complete medium and DMEM low glucose medium (LTI) supplemented with Glutamax I (end concentration 4 mM, LTI) and glucose (end concentration 4.5 gr/L, LTI) and 10% FBS (LTI). For transfection ˜5×10⁵ cells were plated in 10 cm tissue culture dishes. The day after, cells were transfected with 20 μgr of circular pIG270/dish using the CaPO₄ transfection kit (LTI) according to the manufacturer's instructions and cells were incubated overnight with the DNA precipitate. The following day, cells were washed four times with PBS to remove the precipitate and further incubated for over three weeks until foci of transformed cells appeared. Once a week, the medium was replaced by fresh medium. Other transfection agents like, but not limited to, LipofectAmine (LTI) or PEI (Polyethylenimine, high molecular weight, water-free, Aldrich) were used. Of these three agents, PEI reached the best transfection efficiency on primary human amniocytes: ˜1% blue cells 48 hours. Following transfection of pAdApt35.LacZ.

Foci are isolated as follows. The medium is removed and replaced by PBS after which foci are isolated by gently scraping the cells using a 50-200 μl Gilson pipette with a disposable filter tip. Cells contained in ˜10 μl PBS were brought in a 96-well plate containing 15 μl trypsin/EDTA (LTI) and a single cell suspension was obtained by pipetting up and down and a short incubation at room temperature. After addition of 200 μl of the above-described 1:1 mixture of AmnioMax complete medium and DMEM with supplements and 10% FBS, cells were further incubated. Clones that continued to grow were expanded and analyzed for their ability to complement growth of E1-deleted adenoviral vectors of different sub-groups, specifically ones derived from B-group viruses specifically from Ad35 or Ad11.

Generation of New Packaging Cell Lines from HER Cells

HER cells are isolated and cultured in DMEM medium supplemented with 10% FBS (LTI). The day before transfection, ˜5×10⁵ cells are plated in 6 cm dishes and cultured overnight at 37° C. and 10% CO₂. Transfection is done using the CaPO₄ precipitation kit (LTI) according to the manufacturer's instructions. Each dish is transfected with 8 to 10 μgr pIG270 DNA, either as a circular plasmid or as a purified fragment. To obtain the purified fragment, pIG270 was digested with AvrII and XbaI and the 4 kb fragment corresponding to the Ad35 E1 expression cassette was isolated from gel by agarase treatment (Roche). The following day, the precipitate is washed away carefully by four washes with sterile PBS. Then fresh medium is added and transfected cells are further cultured until foci of transformed cells appear. When large enough (>100 cells) foci are picked and brought into 96-well plates as described above. Clones of transformed HER cells that continue to grow are expanded and tested for their ability to complement growth of E1-deleted adenoviral vectors of different sub-groups, specifically ones derived from B-group viruses specifically from Ad35 or Ad11.

New Packaging Cell Lines Derived from PER. C6

As described in Example 8, it is possible to generate and grow Ad35 E1-deleted viruses on PER.C6 cells with co-transfection of an Ad35-E1 expression construct, e.g., pRSV.Ad35.E1. However, large-scale production of recombinant adenoviruses using this method is cumbersome because, for each amplification step, a transfection of the Ad35-E1 construct is needed. In addition, this method increases the risk of non-homologous recombination between the plasmid and the virus genome with high chances of generation of recombinant viruses that incorporate E1 sequences resulting in replication-competent viruses. To avoid this, the expression of Ad35-E1 proteins in PER.C6 has to be mediated by integrated copies of the expression plasmid in the genome. Since PER.C6 cells are already transformed and express Ad5-E1 proteins, addition of extra Ad35-E1 expression may be toxic for the cells; however, it is not impossible to stably transfect transformed cells with E1 proteins since Ad5-E1-expressing A549 cells have been generated.

In an attempt to generate recombinant adenoviruses derived from subgroup B virus Ad7, Abrahamsen et al., 1997, were not able to generate E1-deleted viruses on 293 cells without contamination of wt Ad7. Viruses that were picked after plaque purification on 293-ORF6 cells (Brough et al., 1996) were shown to have incorporated Ad7 E1B sequences by non-homologous recombination. Thus, efficient propagation of Ad7-recombinant viruses proved possible only in the presence of Ad7-E1B expression and Ad5-E4-ORF6 expression. The E1B proteins are known to interact with cellular as well as viral proteins (Bridge et al., 1993; White, 1995). Possibly, the complex formed between the E1B 55K protein and E4-ORF6, which is necessary to increase mRNA export of viral proteins and to inhibit export of most cellular mRNAs, is critical and in some way serotype specific. The above experiments suggest that the E1A proteins of Ad5 are capable of complementing an Ad7-E1A deletion and that Ad7-E1B expression in adenovirus packaging cells on itself is not enough to generate a stable complementing cell line. To test whether one or both of the Ad35-E1B proteins is/are the limiting factor in efficient Ad35 vector propagation on PER.C6 cells, we have generated an Ad35 adapter plasmid that does contain the E1B promoter and E1B sequences but lacks the promoter and the coding region for E1A. Hereto, the left end of wtAd35 DNA was amplified using the primers 35F1 and 35R4 (both described in Example 7) with Pwo DNA polymerase (Roche) according to the manufacturer's instructions. The 4.6 kb PCR product was purified using the PCR purification kit (LTI) and digested with SnaBI and ApaI enzymes. The resulting 4.2 kb fragment was then purified from gel using the QIAExII kit (Qiagen). Next, pAdApt35IP1 (Example 7) was digested with SnaBI and ApaI and the 2.6 kb vector-containing fragment was isolated from gel using the GeneClean kit (BIO 101, Inc). Both isolated fragments were ligated to give pBr/Ad35.leftITR-pIX. Correct amplification during PCR was verified by a functionality test as follows: The DNA was digested with BstBI to liberate the Ad35 insert from vector sequences and 4 μgr of this DNA was co-transfected with 4 μgr of NotI-digested pWE/Ad35.pIX-rITR (Example 7) into PER.C6 cells. The transfected cells were passaged to T80 flasks at day 2 and again two days later; CPE had formed showing that the new pBr/Ad35.leftITR-pIX construct contains functional E1 sequences. The pBr/Ad35.leftITR-pIX construct was then further modified as follows. The DNA was digested with SnaBI and HindIII and the 5′ HindIII overhang was filled in using Klenow enzyme. Religation of the digested DNA and transformation into competent cells (LTI) gave construct pBr/Ad35leftITR-pIXΔE1A. This latter construct contains the left end 4.6 kb of Ad35 except for E1A sequences between bp 450 and 1341 (numbering according to wtAd35 (SEQ ID NO:82)) and thus lacks the E1A promoter and most of the E1A-coding sequences. pBr/Ad35.leftITR-pIXΔE1A was then digested with BstBI and 2 μgr of this construct was co-transfected with 6 μgr of NotI-digested pWE/Ad35.pIX-rITR (Example 7) into PER.C6 cells. One week following transfection, full CPE had formed in the transfected flasks.

This experiment shows that the Ad35-E1A proteins are functionally complemented by Ad5-e1A expression in PER.C6 cells and that at least one of the Ad35-E1B proteins cannot be complemented by Ad5-E1 expression in PER.C6. It further shows that it is possible to make a complementing cell line for Ad35 E1-deleted viruses by expressing Ad35-E1B proteins in PER.C6. Stable expression of Ad35-E1B sequences from integrated copies in the genome of PER.C6 cells may be driven by the E1B promoter and terminated by a heterologous poly-adenylation signal like, but not limited to, the HBVpA. The heterologous pA signal is necessary to avoid overlap between the E1B insert and the recombinant vector, since the natural E1B termination is located in the pIX transcription unit that has to be present on the adenoviral vector. Alternatively, the E1B sequences may be driven by a heterologous promoter like, but not limited to, the human PGK promoter or by an inducible promoter like, but not limited to, the 7xtetO promoter (Gossen and Bujard, 1992). Also in these cases, the transcription termination is mediated by a heterologous pA sequence, e.g., the HBV pA. The Ad35-E1B sequences at least comprise one of the coding regions of the E1B 21K and the E1B 55K proteins located between nucleotides 1611 and 3400 of the wt Ad35 sequence. The insert may also include (part of the) Ad35-E1B sequences between nucleotides 1550 and 1611 of the wt Ad35 sequence.

### Example 15

Generation of Producer Cell Lines for the Production of Recombinant Adenoviral Vectors Deleted in Early Region 1 and Early Region 2A

Herein is described the generation of PER.C6-tTA cell lines for the production of recombinant adenoviral vectors that are deleted in early region 1 (E1) and early region 2A (E2A). The producer cell lines complement for the E1 and E2A deletion from recombinant adenoviral vectors in trans by constitutive expression of both E1 and E2A genes. The pre-established Ad5-E1transformed human embryo retinoblast (“HER”) cell line PER.C6 (International Patent Appln. WO 97/00326) was further equipped with E2A expression cassettes.

The adenoviral E2A gene encodes a 72 kDa DNA Binding Protein that has a high affinity for single-stranded DNA. Because of its function, constitutive expression of DBP is toxic for cells. The ts125E2A mutant encodes a DBP that has a Pro→Ser substitution of amino acid 413. Due to this mutation, the ts125E2A-encoded DBP is fully active at the permissive temperature of 32° C., but does not bind to ssDNA at the non-permissive temperature of 39° C. This allows the generation of cell lines that constitutively express E2A, which is not functional and is not toxic at the non-permissive temperature of 39° C. Temperature-sensitive E2A gradually becomes functional upon temperature decrease and becomes fully functional at a temperature of 32° C., the permissive temperature.

A. Generation of Plasmids Expressing the Wild-Type E2A- or Temperature-Sensitive ts125E2A Gene

pcDNA3wtE2A: The complete wild-type early region 2A- (E2A-) coding region was amplified from the plasmid pBR/Ad.Bam-rITR (ECACC deposit P97082122) with the primers DBPpcr1 and DBPpcr2 using the Expand™ Long Template PCR system according to the standard protocol of the supplier (Boehringer Mannheim). The PCR was performed on a Biometra Trio Thermoblock, using the following amplification program: 94° C. for 2 minutes, 1 cycle; 94° C. for 10 seconds+51° C. for 30 seconds+68° C. for 2 minutes, 1 cycle; 94° C. for 10 seconds+58° C. for 30 seconds+68° C. for 2 minutes, 10 cycles; 94° C. for 10 seconds+58° C. for 30 seconds+68° C. for 2 minutes with 10 seconds extension per cycle, 20 cycles; 68° C. for 5 minutes, 1 cycle. The primer DBPpcr1: CGG GAT CCG CCA CCA TGG CCA GTC GGG AAG AGG AG (5′ to 3′) (SEQ ID NO:80) contains a unique BamHI restriction site (underlined) 5′ of the Kozak sequence (italic) and start codon of the E2A-coding sequence. The primer DBPpcr2: CGG AAT TCT TAA AAA TCA AAG GGG TTC TGC CGC (5′ to 3′) (SEQ ID NO:81) contains a unique EcoRI restriction site (underlined) 3′ of the stop codon of the E2A-coding sequence. The bold characters refer to sequences derived from the E2A-coding region. The PCR fragment was digested with BamHI/EcoRI and cloned into BamHI/EcoRI-digested pcDNA3 (Invitrogen), giving rise to pcDNA3wtE2A.

pcDNA3tsE2A: The complete ts125E2A-coding region was amplified from DNA isolated from the temperature-sensitive adenovirus mutant H5ts125. The PCR amplification procedure was identical to that for the amplification of wtE2A. The PCR fragment was digested with BamHI/EcoRI and cloned into BamHI/EcoRI-digested pcDNA3 (Invitrogen), giving rise to pcDNA3tsE2A. The integrity of the coding sequence of wtE2A and tsE2A was confirmed by sequencing.

B. Growth Characteristics of Producer Cells for the Production of Recombinant Adenoviral Vectors Cultured at 32-, 37- and 39° C.

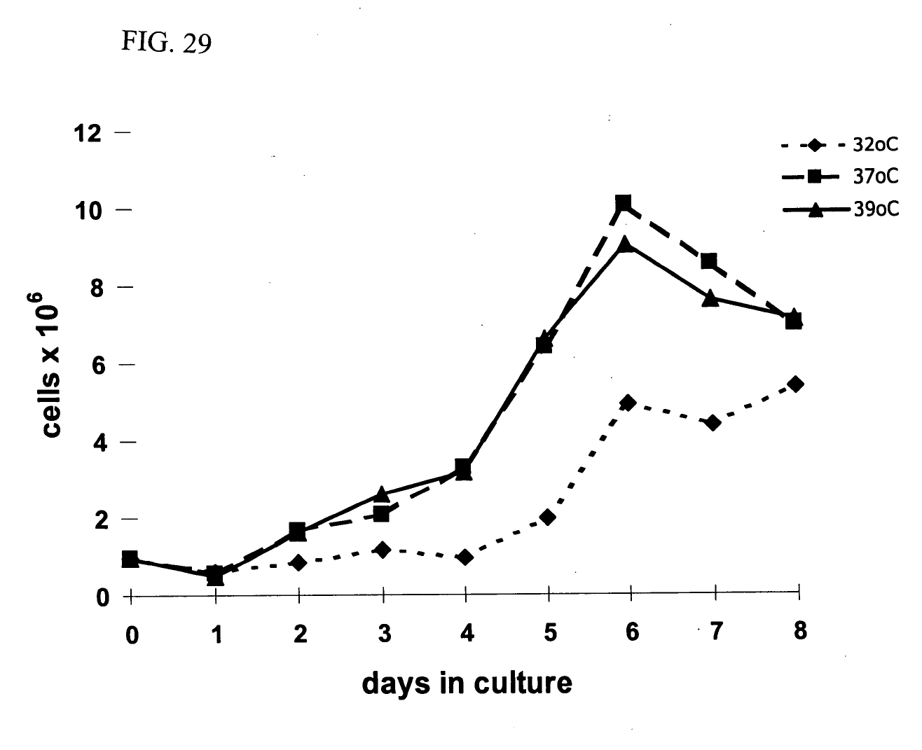

PER.C6 cells were cultured in DMEM (Gibco BRL) supplemented with 10% FBS (Gibco BRL) and 10 mM MgCl₂ in a 10% CO₂ atmosphere at 32° C., 37° C. or 39° C. At day 0, a total of 1×10⁶ PER.C6 cells were seeded per 25 cm² tissue culture flask (Nunc) and the cells were cultured at 32° C., 37° C. or 39° C. At days 1 through 8, cells were counted. FIG. 29 shows that the growth rate and the final cell density of the PER.C6 culture at 39° C. are comparable to that at 37° C. The growth rate and final density of the PER.C6 culture at 32° C. were slightly reduced as compared to that at 37° C. or 39° C. No significant cell death was observed at any of the incubation temperatures. Thus, PER.C6 performs very well both at 32° C. and 39° C., the permissive and non-permissive temperature for ts125E2A, respectively.

C. Transfection of PER. C6 with E2A Expression Vectors; Colony Formation and Generation of Cell Lines

One day prior to transfection, 2×10⁶ PER.C6 cells were seeded per 6 cm tissue culture dish (Greiner) in DMEM, supplemented with 10% FBS and 10 mM MgCl₂ and incubated at 37° C. in a 10% CO₂ atmosphere. The next day, the cells were transfected with 3, 5 or 8 μg of either pcDNA3, pcDNA3wtE2A or pcDNA3tsE2A plasmid DNA per dish, using the LipofectAMINE PLUS™ Reagent Kit according to the standard protocol of the supplier (Gibco BRL), except that the cells were transfected at 39° C. in a 10% CO₂ atmosphere. After the transfection, the cells were constantly kept at 39° C., the non-permissive temperature for ts125E2A. Three days later, the cells were put in DMEM supplemented with 10% FBS, 10 mM MgCl₂ and 0.25 mg/ml G418 (Gibco BRL), and the first G418-resistant colonies appeared at ten days post-transfection. As shown in Table 1, there was a dramatic difference between the total number of colonies obtained after transfection of pcDNA3 (˜200 colonies) or pcDNA3tsE2A (˜100 colonies) and pcDNA3wtE2A (only four colonies). These results indicate that the toxicity of constitutively expressed E2A can be overcome by using a temperature-sensitive mutant of E2A (ts125E2A) and culturing of the cells at the non-permissive temperature of 39° C.

From each transfection, a number of colonies were picked by scraping the cells from the dish with a pipette. The detached cells were subsequently put into 24-well tissue culture dishes (Greiner) and cultured further at 39° C. in a 10% CO₂ atmosphere in DMEM, supplemented with 10% FBS,10 mM MgCl₂ and 0.25 mg/ml G418. As shown in Table 1, 100% of the pcDNA3-transfected colonies (4/4) and 82% of the pcDNA3tsE2A transfected colonies (37/45) were established to stable cell lines (the remaining eight pcDNA3tsE2A-transfected colonies grew slowly and were discarded). In contrast, only one pcDNA3wtE2A-transfected colony could be established. The other three died directly after picking.

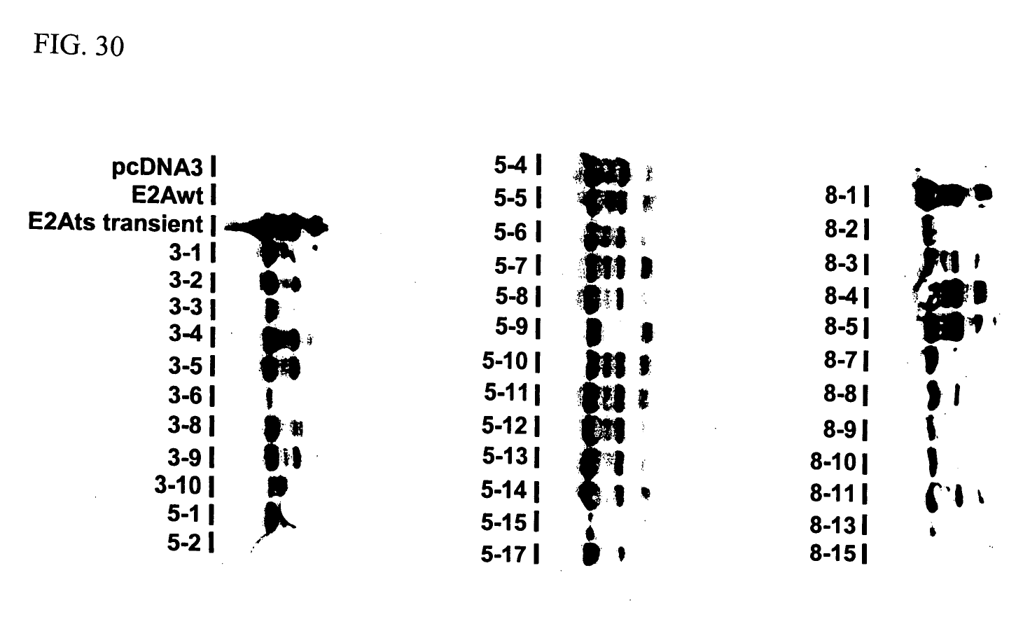

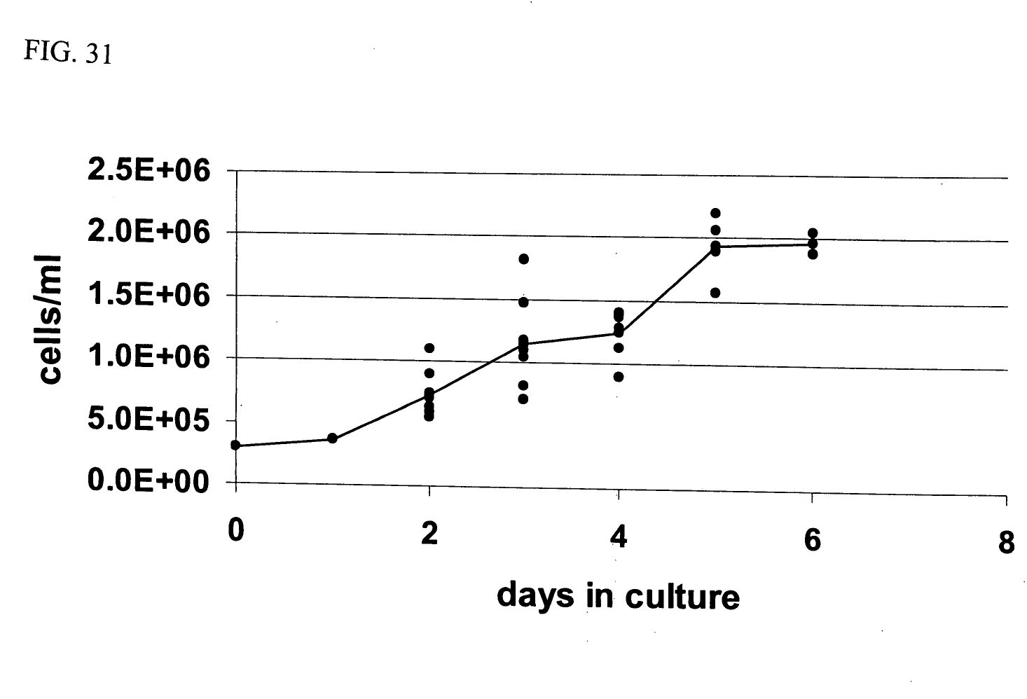

Next, the E2A expression levels in the different cell lines were determined by Western blotting. The cell lines were seeded on 6-well tissue culture dishes and sub-confluent cultures were washed twice with PBS (NPBI) and lysed and scraped in RIPA (1% NP-40, 0.5% sodium deoxycholate and 0.1% SDS in PBS, supplemented with 1 mM phenylmethylsulfonylfluoride and 0.1 mg/ml trypsin inhibitor). After 15 minutes incubation on ice, the lysates were cleared by centrifugation. Protein concentrations were determined by the Bio-Rad protein assay, according to standard procedures of the supplier (BioRad). Equal amounts of whole-cell extract were fractionated by SDS-PAGE on 10% gels. Proteins were transferred onto Immobilon-P membranes (Millipore) and incubated with the αDBP monoclonal antibody B6. The secondary antibody was a horseradish-peroxidase-conjugated goat anti-mouse antibody (BioRad). The Western blotting procedure and incubations were performed according to the protocol provided by Millipore. The complexes were visualized with the ECL-detection system according to the manufacturer's protocol (Amersham). FIG. 30 shows that all of the cell lines derived from the pcDNA3tsE2A transfection expressed the 72-kDa E2A protein (left panel, lanes 4 to 14; middle panel, lanes 1 to 13; right panel, lanes 1 to 12). In contrast, the only cell line derived from the pcDNAwtE2A transfection did not express the E2A protein (left panel, lane 2). No E2A protein was detected in extract from a cell line derived from the pcDNA3 transfection (left panel, lane 1), which served as a negative control. Extract from PER.C6 cells transiently transfected with pcDNA3ts125 (left panel, lane 3) served as a positive control for the Western blot procedure. These data confirmed that constitutive expression of wtE2A is toxic for cells and that using the ts125 mutant of E2A could circumvent this toxicity.

D. Complementation of E2A Deletion in Adenoviral Vectors on PER. C6 Cells Constitutively Expressing Full-Length ts125E2A

The adenovirus Ad5.dl802 is an Ad5-derived vector deleted for the major part of the E2A-coding region and does not produce functional DBP. Ad5.dl802 was used to test the E2A trans-complementing activity of PER.C6 cells constitutively expressing ts125E2A. Parental PER.C6 cells or PER.C6tsE2A clone 3-9 were cultured in DMEM, supplemented with 10% FBS and 10 mM MgCl₂ at 39° C. and 10% CO₂ in 25 cm² flasks and either mock infected or infected with Ad5.dl802 at an m.o.i. of five. Subsequently, the infected cells were cultured at 32° C. and were screened for the appearance of a cytopathic effect (“CPE”) as determined by changes in cell morphology and detachment of the cells from the flask. Full CPE appeared in the Ad5.dl802-infected PER.C6tsE2A clone 3-9 within two days. No CPE appeared in the Ad5.dl802-infected PER.C6 cells or the mock-infected cells. These data showed that PER.C6 cells constitutively expressing ts125E2A complemented in trans for the E2A deletion in the Ad5.dl802 vector at the permissive temperature of 32° C.

E. Serum-Free Suspension Culture of PER. C6tsE2A Cell lines

Large-scale production of recombinant adenoviral vectors for human gene therapy requires an easy and scaleable culturing method for the producer cell line, preferably a suspension culture in medium devoid of any human or animal constituents. To that end, the cell line PER.C6tsE2A c5-9 (designated c5-9) was cultured at 39° C. and 10% CO₂ in a 175 cm² tissue culture flask (Nunc) in DMEM, supplemented with 10% FBS and 10 mM MgCl₂. At sub-confluency (70 to 80% confluent), the cells were washed with PBS (NPBI) and the medium was replaced by 25 ml serum free suspension medium Ex-cell™ 525 (JRH) supplemented with 1× L-Glutamine (Gibco BRL), hereafter designated SFM. Two days later, cells were detached from the flask by flicking and the cells were centrifuged at 1,000 rpm for five minutes. The cell pellet was re-suspended in 5 ml SFM and 0.5 ml cell suspension was transferred to a 80 cm² tissue culture flask (Nunc), together with 12 ml fresh SFM. After two days, cells were harvested (all cells are in suspension) and counted in a Burker cell counter. Next, cells were seeded in a 125 ml tissue culture Erlenmeyer (Corning) at a seeding density of 3×10⁵ cells per ml in a total volume of 20 ml SFM. Cells were further cultured at 125 RPM on an orbital shaker (GFL) at 39° C. in a 10% CO₂ atmosphere. Cells were counted at days 1 through 6 in a Burker cell counter. In FIG. 4, the mean growth curve from eight cultures is shown. PER.C6tsE2A c5-9 performed well in serum-free suspension culture. The maximum cell density of approximately 2×10⁶ cells per ml is reached within five days of culture.

F. Growth Characteristics of PER.C6 and PER. C6/E2A at 37° C. and 39° C.

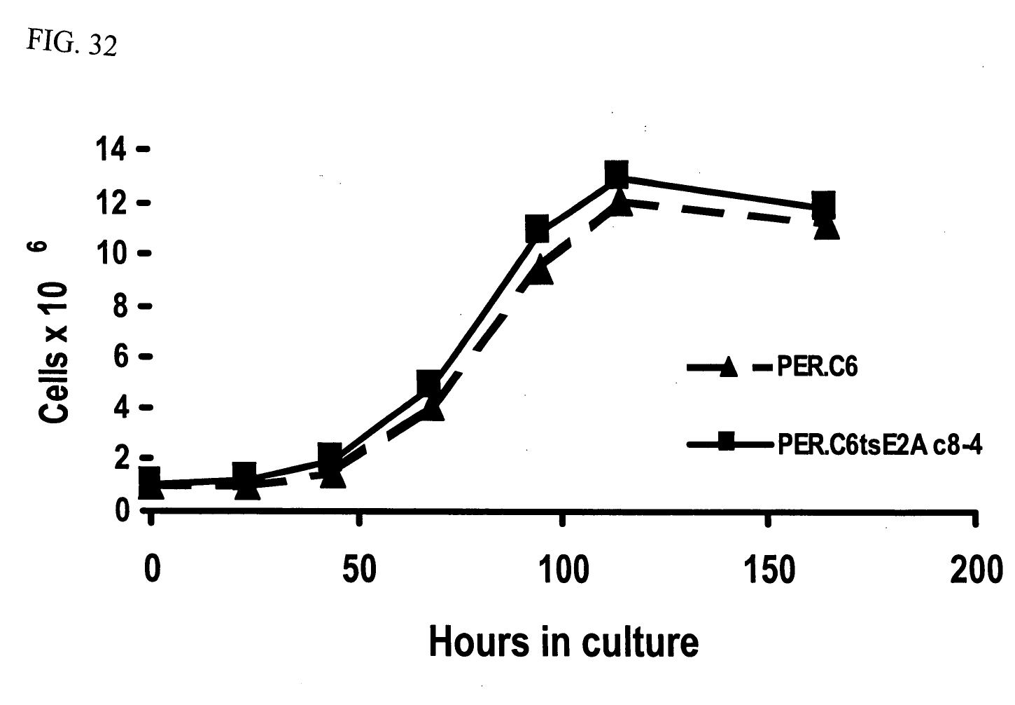

PER.C6 cells or PER.C6ts125E2A (c8-4) cells were cultured in DMEM (Gibco BRL) supplemented with 10% FBS (Gibco BRL) and 10 mM MgCl₂ in a 10% CO₂ atmosphere at either 37° C. (PER.C6) or 39° C. (PER.C6ts125E2A c8-4). At day 0, a total of 1×10⁶ cells were seeded per 25 cm² tissue culture flask (Nunc) and the cells were cultured at the respective temperatures. At the indicated time points, cells were counted. The growth of PER.C6 cells at 37° C. was comparable to the growth of PER.C6ts125E2A c8-4 at 39° C. (FIG. 32). This shows that constitutive expression of ts125E2A-encoded DBP had no adverse effect on the growth of cells at the non-permissive temperature of 39° C.

G. Stability of PER. C6ts125E2A

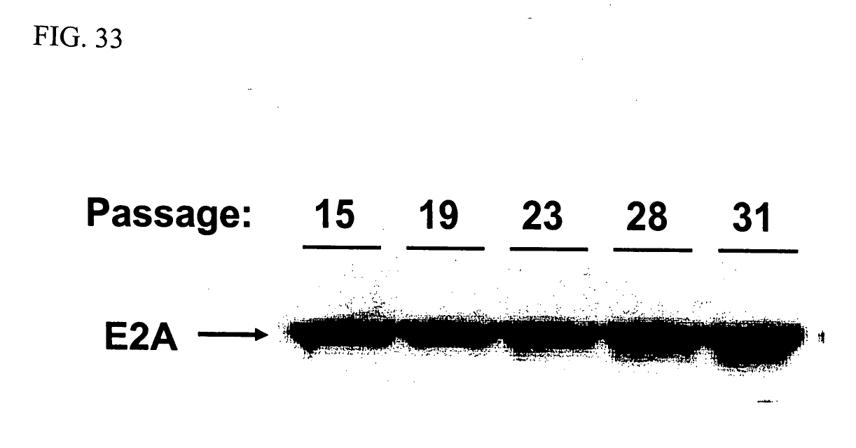

For several passages, the PER.C6ts125E2A cell line clone 8-4 was cultured at 39° C. and 10% CO₂ in a 25 cm² tissue culture flask (Nunc) in DMEM, supplemented with 10% FBS and 10 mM MgCl₂ in the absence of selection pressure (G418). At sub-confluency (70 to 80% confluent), the cells were washed with PBS (NPBI) and lysed and scraped in RIPA (1% NP-40, 0.5% sodium deoxycholate and 0.1% SDS in PBS, supplemented with 1 mM phenylmethylsulfonylfluoride and 0.1 mg/ml trypsin inhibitor). After 15 minutes incubation on ice, the lysates were cleared by centrifugation. Protein concentrations were determined by the BioRad protein assay, according to standard procedures of the supplier (BioRad). Equal amounts of whole-cell extract were fractionated by SDS-PAGE in 10% gels. Proteins were transferred onto Immobilon-P membranes (Millipore) and incubated with the αDBP monoclonal antibody B6. The secondary antibody was a horseradish-peroxidase-conjugated goat anti-mouse antibody (BioRad). The Western blotting procedure and incubations were performed according to the protocol provided by Millipore. The complexes were visualized with the ECL-detection system according to the manufacturer's protocol (Amersham). The expression of ts125E2A-encoded DBP was stable for at least 16 passages, which is equivalent to approximately 40 cell doublings (FIG. 33). No decrease in DBP levels was observed during this culture period, indicating that the expression of ts125E2A was stable, even in the absence of G418 selection pressure.

### Example 16

Generation of tTA-Expressing Packaging Cell Lines

A. Generation of a Plasmid from Which the tTA Gene is Expressed

pcDNA3.1-tTA: The tTA gene, a fusion of the tetR and VP16 genes, was removed from the plasmid pUHD 15-1 (Gossen and Bujard, 1992) by digestion using the restriction enzymes BamHI and EcoRI. First, pUHD15-1 was digested with EcoRI. The linearized plasmid was treated with Klenow enzyme in the presence of dNTPs to fill in the EcoRI sticky ends. Then, the plasmid was digested with BamHI. The resulting fragment, 1025 bp in length, was purified from agarose. Subsequently, the fragment was used in a ligation reaction with BamHI/EcoRV-digested pcDNA 3.1 HYGRO(−) (Invitrogen), giving rise to pcDNA3.1-tTA. After transformation into competent E. Coli DH5α (Life Techn.) and analysis of ampicillin-resistant colonies, one clone was selected that showed a digestion pattern as expected for pcDNA3.1-tTA.

B. Transfection of PER. C6 and PER. C6/E2A with the tTA Expression Vector; Colony Formation and Generation of Cell Lines

One day prior to transfection, 2×10⁶ PER.C6 or PER.C6/E2A cells were seeded per 60 mm tissue culture dish (Greiner) in Dulbecco's modified essential medium (DMEM, Gibco BRL) supplemented with 10% FBS (JRH) and 10 mM MgCl₂ and incubated at 37° C. in a 10% CO₂ atmosphere. The next day, cells were transfected with 4 to 8 μg of pcDNA3.1-tTA plasmid DNA using the LipofectAMINE PLUS™ Reagent Kit according to the standard protocol of the supplier (Gibco BRL). The cells were incubated with the LipofectAMINE PLUS™-DNA mixture for four hours at 37° C. and 10% CO₂. Then, 2 ml of DMEM supplemented with 20% FBS and 10 mM MgCl₂ was added and cells were further incubated at 37° C. and 10% CO₂. The next day, cells were washed with PBS and incubated in fresh DMEM supplemented with 10% FBS, 10 mM MgCl₂ at either 37° C. (PER.C6) or 39° C. (Per.C6/E2A) in a 10% CO₂ atmosphere for three days. Then, the media were exchanged for selection media; PER.C6 cells were incubated with DMEM supplemented with 10% FBS, 10 mM MgCl₂ and 50 μg/ml hygromycin B (GIBCO) while PER.C6/E2A cells were maintained in DMEM supplemented with 10% FBS, 10 mM MgCl₂ and 100 μg/ml hygromycin B. Colonies of cells that resisted the selection appeared within three weeks while nonresistant cells died during this period.

From each transfection, a number of independent, hygromycin-resistant cell colonies were picked by scraping the cells from the dish with a pipette and put into 2.5 cm² dishes (Greiner) for further growth in DMEM containing 10% FBS, 10 mM MgCl₂ and supplemented with 50 μg/ml (PERC.6 cells) or 100 μg/ml (PERC.6/E2A cells) hygromycin in a 10% CO₂ atmosphere and at 37° C. or 39° C., respectively.

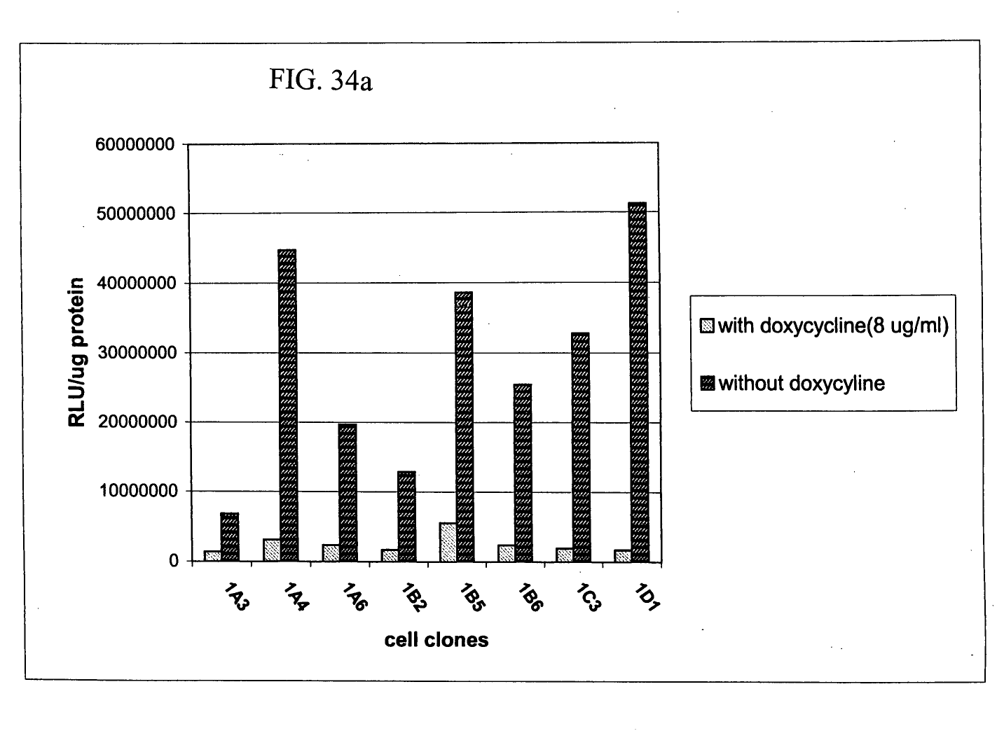

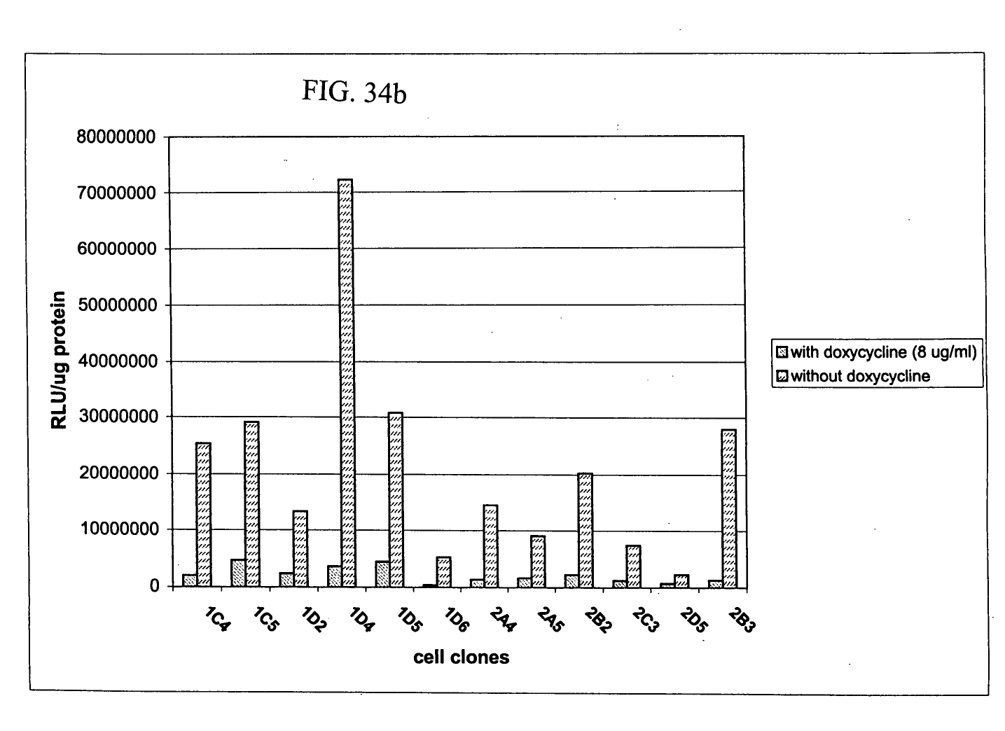

Next, it was determined whether these hygromycin-resistant cell colonies expressed functional tTA protein. Therefore, cultures of PER.C6/tTA or PER/E2A/tTA cells were transfected with the plasmid pUHC 13-3 that contains the reporter gene luciferase under the control of the 7xtetO promoter (Gossens and Bujard, 1992). To demonstrate that the expression of luciferase was mediated by tTA, one-half of the cultures were maintained in medium without doxycycline. The other half was maintained in medium with 8 μg/ml doxycycline (Sigma). The latter drug is an analogue of tetracycline and binds to tTA and inhibits its activity. All PER.C6/tTA and PER/E2A/tTA cell lines yielded high levels of luciferase, indicating that all cell lines expressed the tTA protein (FIG. 34). In addition, the expression of luciferase was greatly suppressed when the cells were treated with doxycycline. Collectively, the data showed that the isolated and established hygromycin-resistant PER.C6 and PER/E2A cell clones all expressed functional tTA.

### Example 17

Sequence of Ad49

The genome sequence of human adenovirus serotype 49 (Ad49) of subgroup D was determined using shot-gun sequencing techniques (Lark, Technologies Inc.). Hereto, Ad49 DNA was isolated from purified virus particles as follows. To 100 μl of virus stock, 12 μl 10× DNAse buffer (130 mM Tris-HCl pH 7.5; 1.2 mM CaCl₂; 50 mM MgCl₂) was added. After addition of 8 μl 10 mg/ml DNAse I (Roche Diagnostics), the mixture was incubated for 1 hour at 37° C. Following addition of 2.5 μl 0.5 M EDTA, 3 μl 20% SDS and 1.5 μl Proteinase K (Roche Diagnostics; 20 mg/ml), samples were incubated at 50° C. for 1 hour. Next, the viral DNA was isolated using the Geneclean spin kit (Bio101 Inc.) according to the manufacturer's instructions. DNA was eluted from the spin column with 25 μl sterile TE.

The obtained Ad49 sequence (35215 nucleotides) is given in SEQ ID NO:87. Comparison with other adenovirus genomes or published fragments thereof reveals the same overall genome structure as known for all human adenoviruses. The overall homology between Ad49 (subgroup D) and Ad35 is 68.1%, which is much lower than the 98.1% homology found between Ad35 and Ad11 viruses (both subgroup B). The homology on the nucleotide level with human Ad9 (Genbank Accession No. AJ854486), which is also a D-group virus, is 93.9%. Table V presents the homology of some of the predicted Ad49 proteins with their Ad9-, Ad5- and Ad35-derived counterparts. Clearly, the homology between the major capsid proteins (penton, hexon and fiber), representing important targets for neutralizing antibodies, is low. The homology between Ad49 and Ad9 is much higher. The E3 region in Ad49, with the coding regions located between nucleotide 26191 and 30743, differs in a number of aspects from the Ad5- and Ad35-E3 regions and has the structure as described for subgroup D viruses (Windheim and Burgert, 2002).

### Example 18

Generation of Recombinant Replication-Deficient Ad49 Viruses

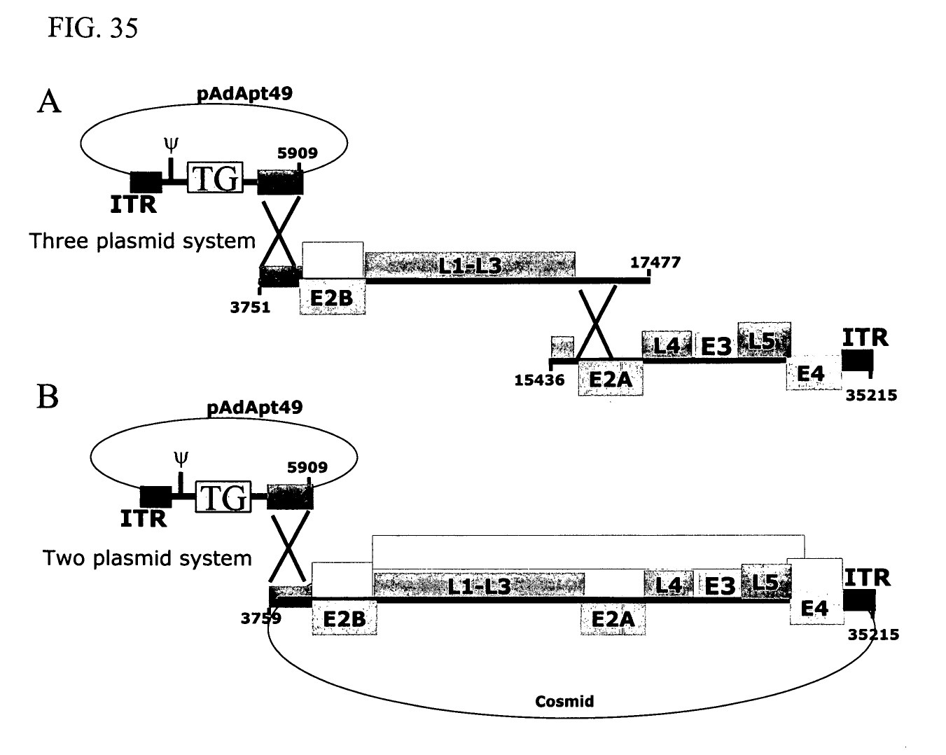

Here, the construction of an Ad49 plasmid-based system to generate recombinant Ad49 vectors in a safe and efficient manner is described. The plasmid system consists of an adapter plasmid containing the Ad49 nucleotides 1 to 461 (including the left ITR and packaging signal), an expression cassette and an Ad49 fragment corresponding to nucleotides 3362 to 5909. The expression cassette comprises a CMV promoter, a multiple cloning site (MCS) and the SV40 poly adenylation signal as described for the recombinant Ad35 and Ad11 vectors (see above). Furthermore, the system consists of one or two other plasmids together constituting Ad49 sequences between nucleotide 3751 and 35215 (see FIG. 35 for the two- or three-plasmid systems that can be applied) that may be deleted for E3 sequences, preferably between nucleotide 26665 to 30735. In addition, the E4-Orf6 and Orf6/7 sequences between 32257 and 33385 are preferably replaced by the corresponding E4 sequences from Ad5. This latter modification ensures efficient replication on Ad5-E1-complementing cell lines, like PER.C6® and 293 cells. The replacement of the E4-Orf6 region of the backbone vector by the E4-Orf6 region of Ad5 (being compatible with the E1B-55K protein produced by the packaging cell) has been described in WO 03/104467, WO 2004/001032, and U.S. Ser. No. 10/512,589, which applications are incorporated herein by reference.

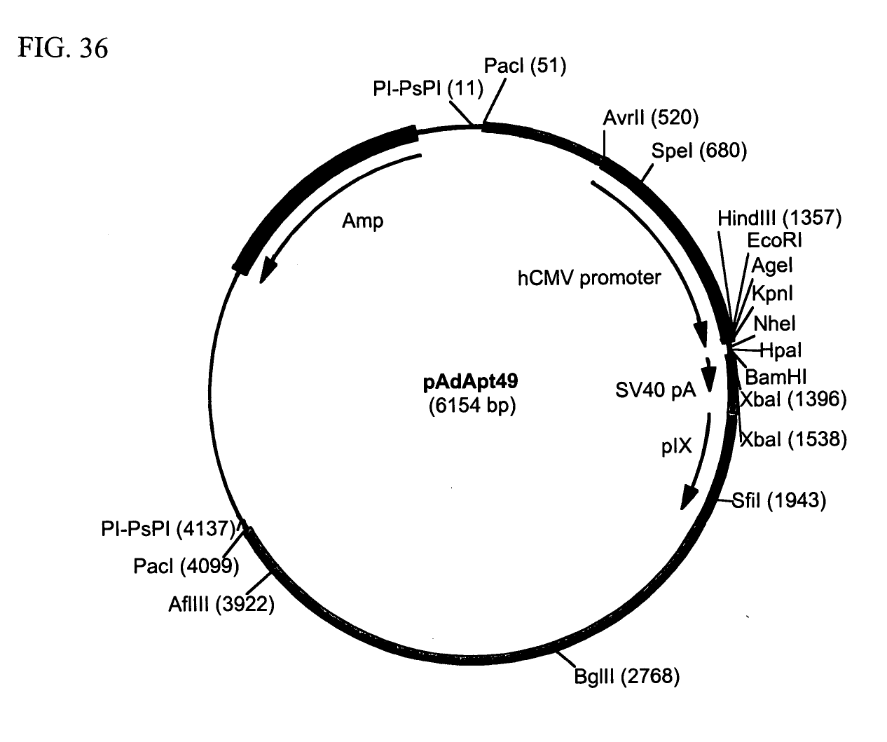

To generate adapter plasmid pAdApt49 (FIG. 36), first a PCR fragment (461 bp) was generated corresponding to Ad49 sequences 1-461 using primers Ad49(1-462)forw 5′-CCT TAA TTA ATC GAC ATC ATC AAT AAT ATA CCC CAC-3′ (SEQ ID NO:88) and Ad49(1-462)rev: 5′-CGC CTA GGT CAG CTG ATC TGT GAC ATA AAC-3′ (SEQ ID NO:89). This PCR introduced a PacI site at the 5′ end and an AvrII site at the 3′ end.

A second PCR fragment (2547 bp) was generated corresponding to Ad49 nucleotides 3362 to 5909 using primers Ad49(3362-5909)forw 5′-CGG GAT CCA GGT AGG TTT TGA GTA GTG GG-3′ (SEQ ID NO:90) and Ad49(3362-5909)rev 5′-CGC GTC GAC TTA ATT AAT CTC GAG AGG GAA TAC CTA C-3′ (SEQ ID NO:91). This PCR introduced a BamHI site at the 5′ end and a PacI and SalI site at the 3′ end of the amplified fragment. Reactions contained 2 μI viral DNA isolated as described above, 0.5 μM of each primer, 0.2 mM dNTP, 1× Pwo polymerase buffer (Roche), 1.25 U Pwo (Roche) and 3% DMSO. The program was set as follows: 4 minutes at 94° C. followed by 30 cycles of 30 seconds at 94° C., 30 seconds at 60° C. and 1 minute at 72° C., and ended by 10 minutes at 68° C.

Both PCR fragments were purified and ligated to TOPO PCR4.1 vector using blunt-end cloning. Following transformation into competent cells, clones were selected containing the correct insert resulting in TOPO.Ad491ITR (461 bp insert) and TOPO.Ad49overlap (2547 bp insert). For the TOPO.Ad491ITR construct, clones were selected that had the AvrII site close to the SpeI site in the TOPO vector. Then plasmid TOPO.Ad491ITR was digested with AvrII and SpeI and the 4.4 kb vector-containing fragment was isolated from gel and dephosphorylated. Plasmid TOPO.Ad49overlap was digested with BamHI and SpeI and the insert fragment was isolated as described above.

Lastly, pAdApt was digested with AvrII and BglII and the 1 kb fragment containing the CMV promoter, MCS and SV40 pA was isolated from gel. The isolated vector fragment and the two inserts were ligated in a molar ratio of 1:3:3 and transformed into competent cells resulting in TOPO.Ad49.AdApt complete. This plasmid was then digested with PacI, Scal and XmnI and the 4 kb AdApt49 insert fragment was isolated from gel. pAdApt35Bsu (see applicant's WO 2004/001032) was digested with PacI, purified from gel, dephosphorylated and ligated to the isolated AdApt49 fragment. Transformation into competent cells resulted in pAdapt49 (FIG. 36): the adapter plasmid containing left-end Ad49 sequences with the E1 region replaced by an expression cassette with the CMV promoter. The E1 deletion includes nucleotide 462-3361 comprising the full E1A and E1B-coding regions.

For the generation of pBrAd49SfiI, a SfiI fragment of Ad49-containing sequences from 3751 to 17477 was sub-cloned in a pBr322-based plasmid. Hereto, a SfiI linker sequence flanked by PacI sites was generated by synthesis of two oligonucleotides sets:

For linker A: SfiI A-up: 5′-CAG AAT TTA ATT AAT GCT GGC CCT GCT GGC CGC TAG CAA TCA G-3′ (SEQ ID NO:92) and SfiI A-do: 5′-CTG ATT GCT AGC GGC CAG CAG GGC CAG CAT TAA TTA AAT TCT G-3′ (SEQ ID NO:93).

For linker B: SfiI B-up: 5′-CAG AAT GCT AGC AAT CAG GGC CGT CAA GGC CGG CAT TAA TTA AGA GAT C-3′ (SEQ ID NO:94) and SfiI B-do: 5′-GAT CTC TTA ATT AAT GCC GGC CTT GAC GGC CCT GAT TGC TAG CAT TCT G-3′ (SEQ ID NO:95).

The oligonucleotides for linker A and for linker B were annealed to give linker A and linker B by mixing 2 μl of a 200 μM stock solution of each A- or B-up oligo with the respective A- or B-do oligo in 1× NEB2 buffer solution in 50 μl volume. The mixtures were then incubated at 100° C. for five minutes followed by a controlled decline in temperature in a thermocycler at 0.02° C./minute to 45° C. after which the tubes were cooled down by incubation on ice.

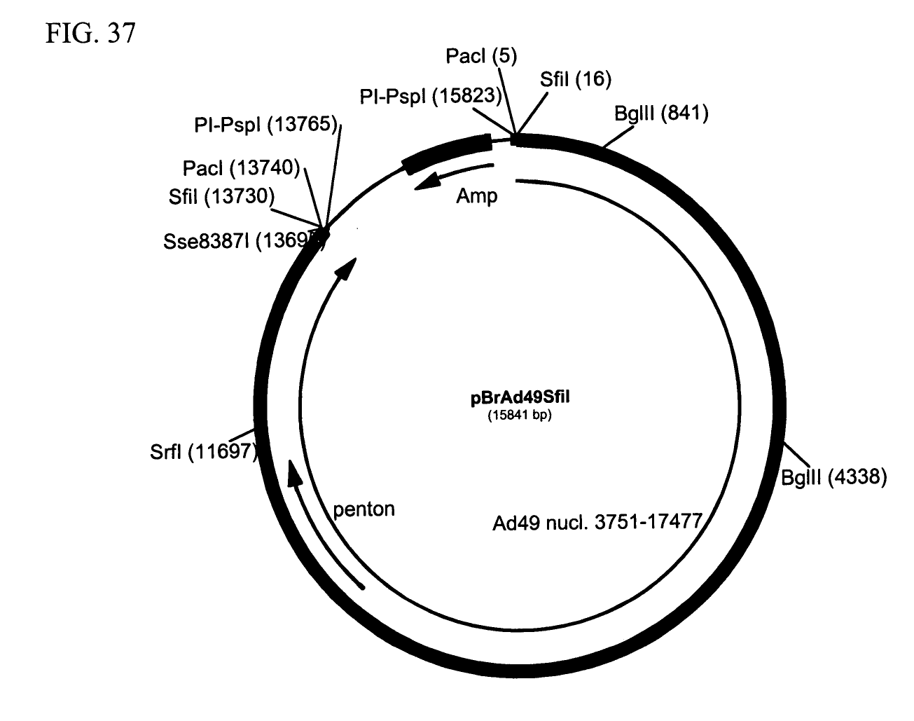

The linkers were then digested with SfiI and NheI and dephosphorylated. Treated linkers were separated from small end fragments using a nucleotide removal kit (Qiagen). In parallel, Ad49 DNA was isolated as described above. DNA was digested with SfiI and KpnI followed by purification of the 13.7 kb SfiI fragment from low-melting point agarose gel using the Zymoclean Gel DNA recovery kit (Zymo Research). The isolated SfiI fragment was ligated to the digested and dephosphorylated linker A and B in a molar excess of ˜200 times of linkers over Ad49 fragment. Following overnight incubation at 16° C., ligase enzyme was inactivated by incubation at 65° C. for 10 minutes and the DNA was subsequently digested with PacI followed by purification using the Zymoclean DNA Clean and Concentrator-5 Kit (Zymo research). The purified 13.7 kb Ad49 SfiI fragment with PacI linkers was then ligated to the vector fragment obtained after PacI digestion of pAdApt35Bsu and purification of the vector backbone from gel and subsequent dephosphorylation. Ligation mixture was incubated at 16° C. overnight and 2 μl of the reaction was then electroporated into competent bacteria. After plating, clones were allowed to grow and analyzed for presence of the correct insert. This resulted in plasmid pBrAd49SfiI (FIG. 37).

Plasmid pBrAd49.Srf-rITR contains Ad49 sequences from the SrfI site at nucleotide 15436 to the end of the right inverted repeat (rITR). To enable cloning of this fragment first, two PCR fragments were generated:

Fragment 1 (2.1 kb) was obtained using the following primers: SrfI-F: 5′-CAG AAT TTA ATT AAA CTA TGC CAG ACG CAA GAG C-3′ (SEQ ID NO:96) and SbfI-R: 5′-CTC GTA CGA GGG CGG CTC-3′ (SEQ ID NO:97). The PCR introduces aPac site at the 5′ end.

Fragment 2 (1.5 kb) was obtained using MluI-F: 5′-CAG AAT CCT GCA GGC TCT ACG CGT ACA TCC AG-3′ (SEQ ID NO:98) and rITR-R: 5′-CAG AAT TTA ATT AAC ATC ATC AAT AAT ATA CCC CAC-3′ (SEQ ID NO:99). This PCR introduces a SbfI site at the 5′ end and a PacI site at the 3′ end.

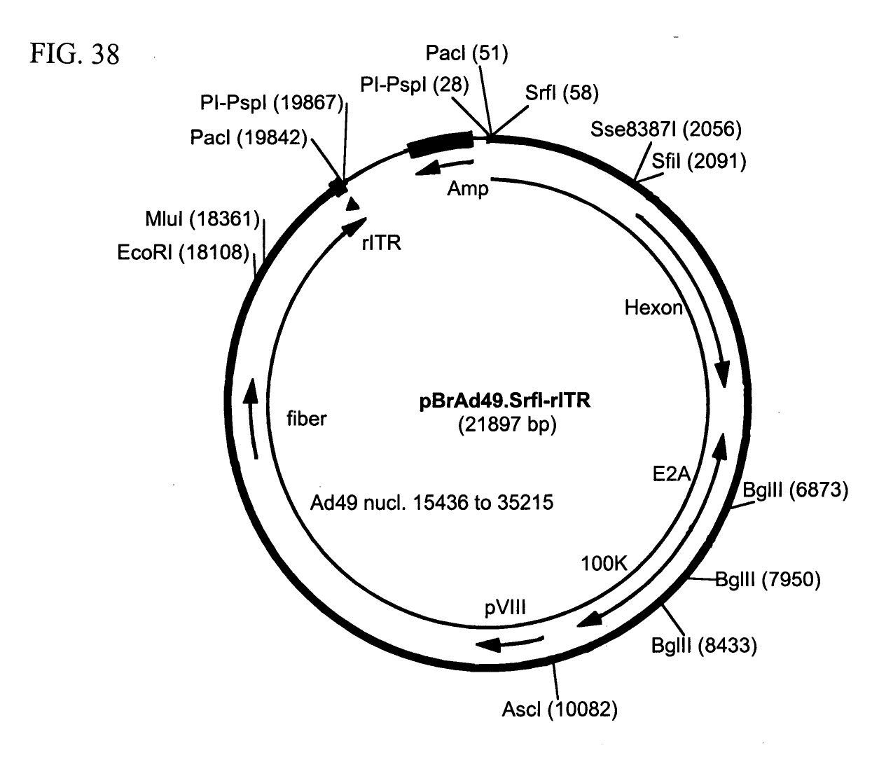

Reactions were done on viral DNA with Phusion DNA polymerase (Finnzymes) with addition of DMSO to a final concentration of 3%. The program was set at 98° C. for 30 seconds followed by 30 cycles of 94° C. for 10 seconds, 58° C. for 10 seconds and 72° C. 45 seconds and ended by 5 minutes at 72° C. PCR products were purified, mixed in approximate equimolar ratio and digested with PacI enzyme followed by purification. pAdApt35Bsu was digested with PacI and the vector was purified and dephosphorylated. This isolated vector was then ligated to the purified PacI-digested PCR fragments in a vector to insert molar ratio of 1:˜25 in the presence of 1 mM rATP and 400 Weiss units ligase (NEB) for 20 minutes at room temperature, after which ligase was heat-inactivated. Next, the ligation mixture was digested with SbfI and the DNA was purified. The cleaned DNA was then ligated at 16° C. overnight and transformed into competent cells. This resulted in plasmid pBrAd49SrfI/SbfI-MluI/rITR that contains Ad49 nucleotides 15365 to 17439 linked to Ad49 nucleotides 33738 to 35215 (end of rITR). pBrAd49SrfI/SbfI-MluI/rITR was then digested with SbfI and MluI, after which the double-digested fragment was isolated from gel. The isolated DNA fragment was then dephosphorylated. In parallel, Ad49 wt DNA was digested with MluI, Sbfl and SanDI. SanDI digests the unwanted Ad49 fragments in order to facilitate easy separation by electrophoresis. The 16.3 kb SbfI-MluI fragment was isolated from gel. This fragment was then ligated to the SbfI-MluI-digested pBrAd49SrfI/Sbfl-MluI/rITR vector and transformed into competent bacteria. This resulted in pBrAd49.SrfI-rITR (FIG. 38).

Plasmid pBrAd49SrfI-rITR was modified to delete part of the E3 region to enlarge the cloning capacity. Hereto, two PCR fragments were generated:

The first PCR fragment (Ad49.AscI/AflII, 1.2 kb) was generated using primer set dE3AscI-F1: 5′-AAA GAC TAA GGC GCG CCC AC-3′ (SEQ ID NO:100) with dE3AflII-R1: 5′-CAG AAT CTT AAG ACG GGT ATT GAC AAC AGC GAG-3′ (SEQ ID NO:101).

The second PCR fragment (Ad49.AflII/EcoRI, 2.8 kb) was generated using primer set: dE3AflII-F2: 5′-CAG AAT CTT AAG CCA TGA ACT AAT GTT GAT TAA AAG-3′ (SEQ ID NO:102) with dE3EcoRI-R2: 5′-GAG GGG AAT TCG CAT GGA CG-3′ (SEQ ID NO:103).

For both fragments, Ad49 wt DNA (˜5 ng/reaction) served as template. Reactions were done using Phusion DNA polymerase (Finnzymes) as described above. The PCR program was set at 30 seconds at 98° C. followed by 30 cycles of 10 seconds at 98° C., 10 seconds at 58° C. and 45 seconds at 72° C., and ended by 5 minutes at 72° C. The PCR products were purified. Next, approximate equimolar amounts of the Ad49.AscI/AflII and Ad49.AflII/EcoRI fragments were mixed and digested with AflII enzyme followed by purification. Next, the digested DNA fragments were ligated after which the ligase enzyme was heat-inactivated. Next, the ligated fragment was digested with AscI and EcoRI followed by purification. Plasmid pBrAd49SrfI-rITR (see above) was then also digested with AscI and EcoRI and the vector-containing fragment was isolated and dephosphorylated. The ligated and digested Ad49.AscI/AflII-Ad49.AflII/EcoRI fragment was then ligated to the isolated vector and the ligation mixture was digested with SpeI to reduce possible background (SpeI linearizes plasmids containing the E3 region). The mixture was then transformed into competent bacteria resulting in plasmid pBRAd49.SrfI-rITR.dE3. This plasmid contains Ad49 sequences from nucleotide 15436 (SrfI site) to the end of the rITR but has a deletion of over 4 kb in the E3 region corresponding to Ad49 nucleotides 26666 to 30735.

To allow efficient generation of recombinant Ad49 vectors on Ad5-transformed cell lines (such as PER.C6 cells) and thus following the technology described in WO 03/104467, construct pBrAd49SrfI-rITR.dE3 was further modified to contain E4-Orf6 and partial Orf6/7 sequences from Ad5 replacing the corresponding sequences in Ad49. Hereto, three PCR fragments were first generated and then assembled:
Fragment 1 (2.75 kb) was generated using primers dE3AscI-F1 (see above), and Ad49-E4orf7-R: 5′-GGG AGA AAG GAC TGT TTA CAC TGT GAA ATG G-3′ (SEQ ID NO: 104).
Fragment 2 (1135 bp) was generated using primers Ad5E4orf6-F: 5′-CAC AGT GTA AAC AGT CCT TTC TCC CCG GCT-3′ (SEQ ID NO:105) and Ad5E4orf6/7-R: 5′-CGG CAG CAG CGA AAT GAC TAC GTC CGG CG-3′ (SEQ ID NO:106).
Fragment 3 (122 bp) was generated using primers Ad49.E4orf4-F:

5′-GGA CGT AGT CAT TTC GCT GCT GCC GCT CAG-3′ (SEQ ID NO:107) and dE3EcoRI-R2 (see above).

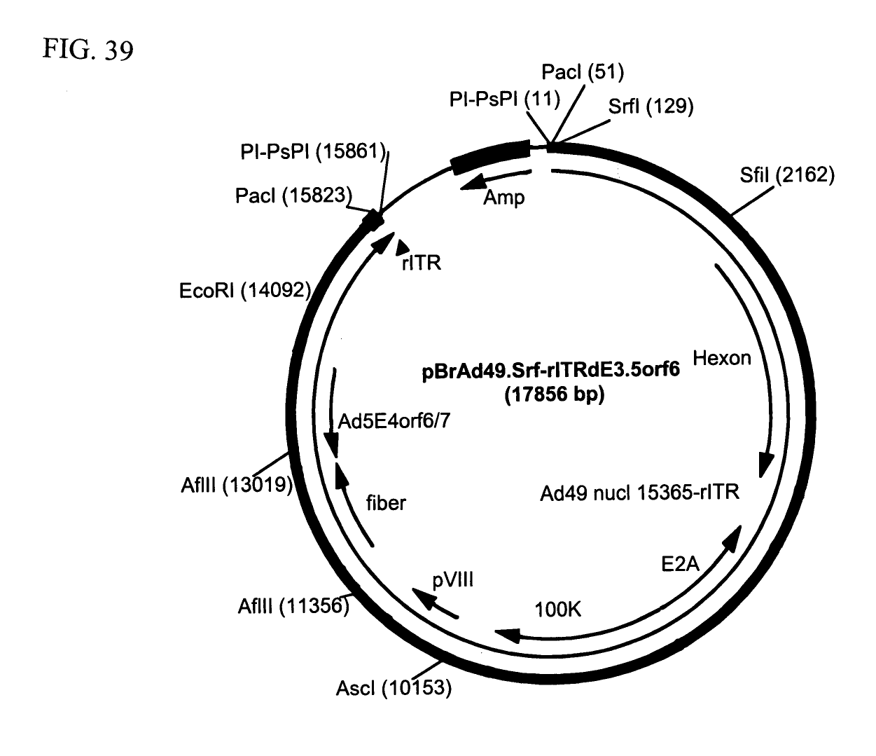

For the amplification of fragments 1 and 3, pBrAd49SrfI-rITR.dE3 was used as template and for the amplification of fragment 2, pBr.Ad35.PRn.dE3.dE4.5Orf6 was used as template. This latter plasmid is identical with respect to the relevant E4 sequences to pBr.Ad35.dE3.PR5orf6 described in WO 03/104467. All reactions were done with Phusion DNA polymerase with addition of DMSO to a final concentration of 3%. The PCR program was set at 30 seconds at 98° C. followed by 30 cycles of 10 seconds at 98° C., 10 seconds at 58° C. and 45 seconds at 72° C. and ended with 5 minutes at 72° C. The amplified products were then isolated from gel. Purified fragments were then mixed in approximate equimolar amounts and, in the presence of the outer border primers dE3AscI-F1 and dE3EcoRI-R2, subjected to an assembly PCR using Phusion DNA polymerase. The program was set at 98° C. for 30 seconds, followed by 30 cycles of 98° C. for 10 seconds, 58° C. for 30 seconds, and 3 minutes at 72° C. and ended by 8 minutes at 72° C. This resulted in a fused PCR product since fragment 2 is at the 5′ and 3′ ends flanked by sequences that have overlap with fragments 1 and 3, respectively. The amplified fragment was purified from gel as above and digested by AscI and EcoRI followed by purification. Plasmid pBrAd49SrfI-rITR was also digested with AscI and EcoRI and the vector-containing fragment was purified from gel followed by dephosphorylation. The assembled and digested PCR fragment was ligated overnight with the digested pBrAd49SrfI-rITR vector, incubated at 65° C. for 15 minutes and digested with SpeI to linearize E3-containing vectors by addition of 1 μl NEB, 1 μl buffer, 8 μl H₂O and 1 μl SpeI. After incubation at 37° C., this mixture was transformed into competent bacteria resulting in plasmid pBrAd49SrfI-rITRdE3.5Orf6 (FIG. 39).

The two described Ad49 fragments in plasmids pBrAd49SfiI and pBrAd49SrfI-rITRdE3.5Orf6 were then combined in a cosmid-based vector to make generation of recombinant viruses even more efficient, requiring one homologous recombination instead of two to reconstitute a full recombinant genome with two ITRs and all genes necessary for replication in a Ad5-E1-transformed cell line (see FIG. 35 for the difference between the two- and three-plasmid systems). Hereto, construct pBrAd49SrfI-rITRdE3.5Orf6 was digested with PacI and SbfI and the resulting 2.6 kb SbfI-PacI fragment (including the right ITR), and the 11 kb SbfI fragment were isolated from gel. Furthermore, construct pBr.Ad49SfiI was digested with PacI and Sbfl, and the 13.7 kb PacI/SbfI fragment was isolated. Lastly, construct pWE.Ad5.AflII-rITR.dE3 (described in applicant's WO 99/55132), was digested with PacI and the cosmid vector was isolated in the same way. The purified cosmid vector was dephosphorylated, followed by heat inactivation. The isolated pWE vector was then ligated with the 2.6 kb SbfI-PacI fragment and the 13.7 kb PacI/SbfI fragment and transformed into electrocompetent bacteria to give construct pWEAd49pIX-rITR.dE3.5Orf6.d11SBF. This construct was linearized with SbfI enzyme and the DNA was isolated from gel, followed by dephosphorylation. The above-described purified 11.1 kb SbfI fragment was then ligated to the SbfI-digested pWEAd49pIX-rITR.dE3.5Orf6.d11 SBF fragment and transformed into electrocompetent bacteria, resulting in pWE.Ad49pIX-rITR.dE3.5Orf6.

Small-Scale Production of Ad49dE3.5Orf6.Luc Viruses

As an example of the generation and production of replication-deficient recombinant Ad49-based viruses that express a foreign (heterologous) transgene, the production of Ad49-based luciferase viruses is described here. Plasmid pAdApt49.Luc was digested with PI-PsPI, while plasmids pBrAd49SfiI and pBrAd49SrfI-rITRdE35Orf6 were digested with PacI enzyme to liberate the Ad49 sequences from the vector backbone. The day before transfection, 3.5×10⁶ PER.C6 cells were seeded in T25 flasks. Transfection was done with a total of 8 μg DNA (2:3:3 ratio for pAdApt49.Luc versus the two backbone constructs) with 40 μl Lipofectamine. Two days after the transfection, cells were passed to a T75 culture flask. After two cycles of re-infection of harvested and freeze/thawed material on fresh PER.C6 cells, full CPE crude lysate was used to perform a plaque assay by serial dilution (10⁻⁴ to 10⁻⁹). Single plaques were picked and propagated in 24-well plates. The day before infection, 2.5×10⁵ PER.C6 cells were seeded in the 24-well plate. Upon full CPE, crude lysates were harvested by one freeze/thaw cycle and 100 μl was used to re-infect a fresh culture of PER.C6 cells in a T25 flask seeded the day before at 3.5×10⁶ cells/flask. Next, the Ad49.Luc viruses were propagated to T175 flask and subsequently to 24 triple-layer T175 flasks for small-scale production and purification using a two-step CsCl gradient procedure and dialysis. In this way, approximately 10 ml of 9×10¹¹ virus particles/ml was obtained.

### Example 19

Absence of in vivo Cross-Neutralization Between d49 and Ad5 or Ad35

Ad35 and Ad11, just like Ad49 and others identified infra, are vectors with low prevalence of neutralizing activity in human serum (see also Vogels et al., 2003; Holterman et al., 2004) and are, therefore, good candidates for vaccine vectors in areas where the sero-prevalence to Ad5 is high. However, if Ad35- and Ad11-recombinant vectors are used in combination with each other in mice, e.g., in a prime-boost schedule, the efficacy of the second administered vector is reduced, most likely due to the high homology between the two vectors. Still, in the presence of Ad5 pre-existing immunity, Ad35/Ad11 combinations give better immune induction as compared to Ad35/Ad5 or Ad11/Ad5 combinations. Alternative vectors with less homology to Ad35 may be more efficient as prime-boost vectors. As an example for such an adenoviral vector, it is now shown here that Ad49 is suitable for use in prime-boost combination with Ad35 in the presence of Ad5 pre-existing immunity. Hereto, mice were immunized with 10¹⁰ virus particles (VP) of wild-type (wt) Ad49 once, for low levels of Ad49-specific neutralizing activity (NA), or twice with a 4-week interval for high levels of NA. Pre-immunized mice were then injected with 10⁹ VP of recombinant Ad35 (rAd35) or rAd5 vectors expressing SIV-gag antigens four weeks after the (last) pre-immunization. A direct comparison with naive mice that received 10⁹ VP SIV-gag vectors allowed analysis of cross-neutralization in vivo. FIG. 40 shows the results in mice that were pre-immunized once with Ad49. FIG. 40A presents the anti-Ad49 titers in the mice proving that the pre-immunization resulted in induction of anti-Ad49 immunity in injected mice. Also, two immunizations with wtAd49 resulted in significantly higher anti-Ad49 NA as compared to one injection (FIG. 40A versus FIG. 41A).

FIG. 40B (low pre-existing Ad49 immunity) and FIG. 41B (high pre-existing Ad49 immunity) present the percentages of T-cells that bind to tetramers presenting an SIV-gag peptide (as described in Barough et al., 2004) in blood samples taken 7 to 28 days after injection of the SIV-gag viruses. In both cases, high or low pre-existing Ad49 immunity, neither Ad5 nor Ad35 SIV-gag vectors are significantly inhibited. From this, it is concluded that even in the presence of anti-Ad5 immunity, a situation encountered in half (e.g., U.S.A., see Vogels et al., 2003) or even in more than 90% (African continent; see Kostense et al., 2004) of the human individuals, Ad35 vectors can be used to efficiently boost Ad49-induced immune responses to, e.g., a virus- and/or pathogen-specific antigen or, vice versa, Ad49 vectors can be used to efficiently boost Ad35 induced immunity.

TABLE 1

| | Elution | | | log₁₀ VP/ |
|---|---|---|---|---|
| Serotype | [NaCl] mM | VP/ml | CCID50 | CCID50 ratio |
| 1 | 597 | 8.66 × 10¹⁰ | 5.00 × 10⁷ | 3.2 |
| 2 | 574 | 1.04 × 10¹² | 3.66 × 10¹¹ | 0.4 |
| 3 | 131 | 1.19 × 10¹¹ | 1.28 × 10⁷ | 4.0 |
| 4 | 260 | 4.84 × 10¹¹ | 2.50 × 10⁸ | 3.3 |
| 5 | 533 | 5.40 × 10¹¹ | 1.12 × 10¹⁰ | 1.7 |
| 6 | 477 | 1.05 × 10¹² | 2.14 × 10¹⁰ | 1.7 |
| 7 | 328 | 1.68 × 10¹² | 2.73 × 10⁹ | 2.4 |
| 9 | 379 | 4.99 × 10¹¹ | 3.75 × 10⁷ | 4.1 |
| 10 | 387 | 8.32 × 10¹² | 1.12 × 10⁹ | 3.9 |
| 12 | 305 | 3.64 × 10¹¹ | 1.46 × 10⁷ | 4.4 |
| 13 | 231 | 4.37 × 10¹² | 7.31 × 10⁸ | 3.8 |
| 15 | 443 | 5.33 × 10¹² | 1.25 × 10⁹ | 3.6 |
| 16 | 312 | 1.75 × 10¹² | 5.59 × 10⁸ | 3.5 |
| 17 | 478 | 1.39 × 10¹² | 1.45 × 10⁹ | 3.0 |
| 19 | 430 | 8.44 × 10¹¹ | 8.55 × 10⁷ | 4.0 |
| 20 | 156 | 1.41 × 10¹¹ | 1.68 × 10⁷ | 3.9 |
| 21 | 437 | 3.21 × 10¹¹ | 1.12 × 10⁸ | 3.5 |
| 22 | 365 | 1.43 × 10¹² | 5.59 × 10⁷ | 3.4 |
| 23 | 132 | 2.33 × 10¹¹ | 1.57 × 10⁷ | 4.2 |
| 24 | 405 | 5.12 × 10¹² | 4.27 × 10⁸ | 4.1 |
| 25 | 405 | 7.24 × 10¹¹ | 5.59 × 10⁷ | 4.1 |
| 26 | 356 | 1.13 × 10¹² | 1.12 × 10⁸ | 4.0 |
| 27 | 342 | 2.00 × 10¹² | 1.28 × 10⁸ | 4.2 |
| 28 | 347 | 2.77 × 10¹² | 5.00 × 10⁷ | 4.7 |
| 29 | 386 | 2.78 × 10¹¹ | 2.00 × 10⁷ | 4.1 |
| 30 | 409 | 1.33 × 10¹² | 5.59 × 10⁸ | 3.4 |
| 31 | 303 | 8.48 × 10¹⁰ | 2.19 × 10⁷ | 3.6 |
| 33 | 302 | 1.02 × 10¹² | 1.12 × 10⁷ | 5.0 |
| 34 | 425 | 1.08 × 10¹² | 1.63 × 10¹¹ | 0.8 |
| 35 | 446 | 3.26 × 10¹² | 1.25 × 10¹¹ | 1.4 |
| 36 | 325 | 9.26 × 10¹² | 3.62 × 10⁹ | 3.4 |
| 37 | 257 | 5.86 × 10¹² | 2.8 × 10⁹ | 3.3 |
| 38 | 337 | 3.61 × 10¹² | 5.59 × 10⁷ | 4.8 |
| 39 | 241 | 3.34 × 10¹¹ | 1.17 × 10⁷ | 4.5 |
| 42 | 370 | 1.95 × 10¹² | 1.12 × 10⁸ | 4.2 |
| 43 | 284 | 2.42 × 10¹² | 1.81 × 10⁸ | 4.1 |
| 44 | 295 | 8.45 × 10¹¹ | 2.00 × 10⁷ | 4.6 |
| 45 | 283 | 5.20 × 10¹¹ | 2.99 × 10⁷ | 4.2 |
| 46 | 282 | 9.73 × 10¹² | 2.50 × 10⁸ | 4.6 |
| 47 | 271 | 5.69 × 10¹¹ | 3.42 × 10⁷ | 4.2 |
| 48 | 264 | 1.68 × 10¹² | 9.56 × 10⁸ | 3.3 |
| 49 | 332 | 2.20 × 10¹² | 8.55 × 10⁷ | 4.4 |
| 50 | 459 | 7.38 × 10¹² | 2.80 × 10⁹ | 3.4 |
| 51 | 450 | 8.41 × 10¹¹ | 1.88 × 10⁸ | 3.7 |

Legend to table I:

All human adenoviruses used in the neutralization experiments were produced on PER.C6 cells (Fallaux et al., 1998) and purified on CsCl as described in Example 1. The NaCl concentration at which the different serotypes eluted from the HPLC column is shown. Virus particles/ml (VP/ml) were calculated from an Ad5 standard. The titer in the experiment (CCID50) was determined on PER.C6 cells as described in Example 1 by titrations performed in parallel with the neutralization experiment. The CCID50 is shown for the 44 viruses used in this study and reflects the dilution of the virus needed to obtain CPE in 50% of the wells after five days. The ratio of VP/CCID50 is depicted in log₁₀ and is a measurement of the infectivity of the different batches on PER.C6 cells.

TABLE II
AdApt35.LacZ viruses escape neutralization by human serum.

| | Human serum dilution | | | | | |
|---|---|---|---|---|---|---|
| Virus | no serum | 10 × | 50 × | 250 × | 1250 × | 6250 × |
| AdApt5.LacZ | 100% | 0% | 0% | 1% | 40% | 80% |
| moi: 5 VP/cell | | | | | | |
| AdApt35.LacZ | 100% | 100% | 100% | 100% | 100% | 100% |
| 250 μl crude lysate | | | | | | |

TABLE III
Percentage of synovial fluid samples containing neutralizing activity
(NA) to wt adenoviruses of different serotypes.

| | % of SF samples with NA | % of SF samples with NA |
|---|---|---|
| | (all positives) | (positives at ≧64 × dilution) |
| Ad5 | 72 | 59 |
| Ad26 | 66 | 34 |
| Ad34 | 45 | 19 |
| Ad35 | 4 | 0 |
| Ad48 | 42 | 4 |

TABLE IV
The numbers of foci obtained with the different E1 expression
constructs in BRK transformation experiments.
Average # of foci/dish:

| | Construct | 1 μgr | 5 μgr |
|---|---|---|---|
| Experiment 1 | pIG.E1A.E1B | nd | 60 |
| | pIG.E1A.E1B | nd | 35 |
| | pRSVAd35E1 | 0 | 3 |
| | pIG.Ad35.E1 | 3 | 7 |
| Experiment 2 | pIG.E1A.E1B | 37 | nd |
| | pIG.Ad35.E1 | nd | 2 |
| Experiment 3 | pIG.E1A.E1B | nd | 140 |
| | pIG.Ad35.E1 | nd | 20 |
| | pIG270 | nd | 30 |

TABLE V
Percentages of homology of the proteins encoded by viruses from
Subgroup C (Ad5), B (Ad35) and D (Ad9) with the corresponding
predicted proteins of Ad49.

| | fiber | penton | hexon | E1B-55K | E4-orf6 |
|---|---|---|---|---|---|
| Ad5 | 45.5 | 76.1 | 78.2 | 52.5 | 59.2 |
| Ad35 | 32.5 | 81.3 | 83.4 | 56.5 | 64.5 |
| Ad9 | 63.5 | 91.9 | 90.1 | 97.8 | 99.3 |

### REFERENCES

Abrahamsen K., H.-L. Kong, A. Mastrangeli, D. Brough, A. Lizonova, R. Crystal and E. Falck-Pedersen (1997) Construction of an adenovirus type 7a E1A⁻ vector. J. Virol. 71, no. 11, p8946-8951.

Athappilly F. K., R. Murali, J. J. Rux, Z. Cai and R. M. Burnett (1994) The refined crystal structure of hexon, the major coat protein of adenovirus type 2, at 2.9 Å resolution. J. Mol. Biol. 242:430-455.

Basler C. F., G. Droguett and M. S. Horwitz (1996) Sequence of the immunoregulatory early region 3 and flanking sequences of adenovirus type 35. Gene 170:249-54.

Barouch D. H., M. G. Pau, J. H. Custers, W. Koudstaal, S. Kostense, M. J. Havenga, D. M. Truitt, S. M. Sumida, M. G. Kishko, J. C. Arthur, B. Korioth-Schmitz, M. H. Newberg, D. A. Gorgone, M. A. Lifton, D. L. Panicali, G. J. Nabel, N. L. Letvin and J. Goudsmit (2004) Immunogenicity of recombinant adenovirus serotype 35 vaccine in the presence of pre-existing anti-Ad5 immunity. J. Immunol. 172:6290-7.

Bridge E., S. Medghalchi, S. Ubol, M. Leesong and G. Ketner (1993) Adenovirus early region 4 and viral DNA synthesis. Virology 193:794-801.

Brody S. L. and R. G. Crystal (1994) Adenovirus mediated in vivo gene transfer. Ann. N.Y. Acad. Sci. 716:90-101.

Boshart M., F. Weber, G. Jahn, K. Dorsch-Hasler, B. Fleckenstein and W. Schaffner (1985) A very strong enhancer is located upstream of an immediate early gene of human cytomegalovirus. Cell 41:521-530.

Dijkema R., B. M. M. Dekker, M. J. M. van der Feltz and A. J. van der Eb (1979) Transformation of primary rat kidney cells by DNA fragments of weakly oncogenic adenoviruses. J. Virol. 32, No. 3, 943-950.

Fallaux F. J., A. Bout, I. van der Velde, D. J. van den Wollenberg, K. M. Hehir, J. Keegan, C. Auger, S. J. Cramer, H. van Ormondt, A. J. van der Eb, D. Valerio and R. C. Hoeben (1998) New helper cells and matched early region 1-deleted adenovirus vectors prevent generation of replication-competent adenoviruses. Hum. Gene Ther. 9:1909-1917.

Flomenberg P. R., M. Chen, G. Munk and M. S. Horwitz (1987) Molecular epidemiology of adenovirus type 35 infections in immunocompromised hosts. J. Infect Dis. 155(6):1127-34.

Francki R. I. B., C. M. Fauquet, D. L. Knudson and F. Brown (1991) Classification and nomenclature of viruses. Fifth report of the international Committee on taxonomy of viruses. Arch. Virol. Suppl. 2:140-144.

Gahery-Segard H., F. Farace, D. Godfiin, J. Gaston, R. Lengagne, P. Tursz, P. Boulanger and J.-G. Guillet (1998) Immune response to recombinant capsid proteins of adenovirus in humans: Anti-fiber and anti-penton base antibodies have a synergistic effect on neutralizing activity. J. Virol. 72:2388-2397.

Gossen M. and H. Bujard (1992) Tight control of gene expression in mammalian cells by tetracycline-responsive promoters. Proc. Natl. Acad. Sci. USA 89:5547-5551.

He T-C., S. Zhou, L. T. Da Costa, J. Yu, K. W. Kinzler and B. Vogelstein (1998) A simplified system for generating recombinant adenoviruses. Proc. Natl. Acad. Sci. USA 95:2509-2514.

Hierholzer J. C., R. Wigand, L. J. Anderson, T. Adrian and J. W. M. Gold (1988) Adenoviruses from patients with AIDS: a plethora of serotypes and a description of five new serotypes of subgenus D (types 43-47). J. Infect. Dis. 158:804-813.

Holterman L., R. Vogels, R. van der Vlugt, M. Sieuwerts, J. Grimbergen, J. Kaspers, E. Geelen, E. Van der Helm, A. Lemckert, G. Gillissen, S. Verhaagh, J. Custers, D. Zuijdgeest, B. Berkhout, M. Bakker, P. Quax, J. Goudsmit and M. Havenga (2004) Novel replication-incompetent vector derived from adenovirus type 11 (Ad11) for vaccination and gene therapy: Low seroprevalence and non-cross-reactivity with Ad5. J. Virol. 78:13207-13215.

De Jong P. J., G. Valderrama, I. Spigland and M. S. Horwitz (1983) Adenovirus isolates from urine of patients with acquired immunodeficiency syndrome. Lancet 1(8337):1293-1296.

Kay R., F. Takei and R. K. Humphries (1990) Expression cloning of a cDNA encoding M1/69. J. Immunol. 145:1952-1959.

Kang W. G., G. Berencsi, M. Takacs, Z. Ascher, G. Fejer, I. Nasz (1989a) Molecular cloning and physical mapping of the DNA of human adenovirus type 35. Acta. Microbiol. Hung. 36(1):67-75.

Kang W. G., G. Berencsi, A. Banrevi, Z. Ascher, G. Fejer, M. Takacs, A. Kiss and I. Nasz (1989b) Relationship of E1 and E3 regions of human Ad35 to those of human adenovirus subgroups A, C and D. Acta. Microbiol. Hung. 36(4):445-57.

Kostense S., W. Koudstaal, M. Sprangers, G. J. Weverling, P. Penders, N. Helmus, R. Vogels, M. Bakker, B. Berkhout, M. Havenga and J. Goudsmit (2004) Adenovirus types 5 and 35 seroprevalence in AIDS risk groups supports type 35 as a vaccine vector: Aids 18:1213-1216.

Levrero M., V. Barban, S. Manteca, A. Ballay, C. Balsamo, M. L. Avantaggiati, G. Natoli, H. Skellekens, P. Tiollais and M. Perricaudet (1991) Defective and nondefective adenovirus vectors for expressing foreign genes in vitro and in vivo. Gene 101:195-202.

Li Q. G., J. Hambraeus and G. Wadell (1991) Genetic relationship between thirteen genomes of types of adenovirus 11, 34, and 35 with different tropisms. Intervirol. 32:338-350.

Prince H. M. (1998) Gene transfer: a review of methods and applications. Pathology 30(4):335-347.

Robbins P. D. and S. C. Ghivizzani (1998) Viral vectors for gene therapy. Pharmacol. Ther. 80:35-47.

Schnurr D. and M. E. Dondero (1993) Two new candidate adenovirus serotypes. Intervirol. 36:79-83.

Schulick A. H., G. Vassalli, P. F. Dunn, G. Dong, J. J. Rade, C. Zamarron and D. A. Dichek (1997) Established immunity precludes adenovirus-mediated gene transfer in rat carotid arteries. Potential for immunosuppression and vector engineering to overcome barriers of immunity. J. Clin. Invest. 99(2):209-19.

Shabram P. W., D. Giroux, A. M. Goudreau, R. J. Gregory, M. Horn, B. G. Huyghe, X. Liu, M. H. Nunnally, B. J. Sugarman and S. Sutjipto (1997) Analytical anion-exchange HPLC of recombinant type-5 adenoviral particles. Hum. Gene. Ther. 8:453-465.

Toogood et al., 1989; J. Gen Virol. 70:3203-3214.

Toogood C. I., R. Murali, R. M. Burnett and R. T. Hay (1989) The adenovirus type 40 hexon: sequence, predicted structure and relationship to other adenovirus hexons. J. Gen. Virol. 70:3203-14.

Valderrama-Leon G., P. Flomenberg and M. S. Horwitz (1985) Restriction endonuclease mapping of adenovirus 35, a type isolated from immunocompromised hosts. J. Virol. 56(2):647-50.

Vogels R., D. Zuijdgeest, R. van Rijnsoever, E. Hartkoom, I. Damen, M.-P. de Béthune, S. Kostense, G. Penders, N. Helmus, W. Koudstaal, M. Cecchini, A. Wetterwald, M. Sprangers, A. Lemckert, O. Ophorst, B. Koel, M. Meerendonk, P. Quax, L. Panitti, J. Grimbergen, A. Bout, J. Goudsmit and M. Havenga (2003) Replication-deficient human adenovirus type 35 vectors for gene transfer and vaccination: Efficient human cell infection and bypass of preexisting adenovirus immunity. J. Virol. 77:8263-8271.

Wadell G. (1984) Molecular epidemiology of adenoviruses. Curr. Top. Microbiol. Immunol. 110:191-220.

White E. (1995) Regulation of p53-dependent apoptosis by E1a and E1b. Curr. Top. Microbiol. Immunol. 199:34-58.

Windheim and Burgert (2002) Characterization of E3/49K, a novel, highly glycosylated E3 protein of the epidemic keratoconjuntivis-causing adenovirus type 19a. J. Virol. 76:755-766.

1. A recombinant replication-defective adenoviral vector based on adenovirus serotype Ad49, said adenoviral vector comprising:
a gene of interest operatively linked to a promoter; and
at least one adenovirus capsid protein from adenovirus serotype Ad11, Ad35, or Ad49.

2. The recombinant replication-defective adenoviral vector of claim 1, wherein said recombinant replication-defective adenoviral vector is a chimera of Ad49 and at least one other adenovirus serotype.

3. The recombinant replication-defective adenoviral vector of claim 1, wherein said recombinant replication-defective adenoviral vector has a different tropism than Ad49.

4. The recombinant replication-defective adenoviral vector of claim 3, wherein said recombinant replication-defective adenoviral vector has one or more tropism-determining parts of adenovirus serotype 16.

5. The recombinant replication-defective adenoviral vector of claim 1, wherein said adenovirus capsid protein comprises an adenovirus fiber protein and a penton or hexon protein, or both said penton and said hexon protein.

6. A pharmaceutical formulation comprising:
a recombinant replication-defective adenoviral vector based on adenovirus serotype Ad49, wherein said recombinant replication-defective adenoviral vector comprises:
at least one adenovirus capsid protein of adenovirus serotype Ad11, Ad35, or Ad49; and
a gene of interest operatively linked to a promoter; and
a suitable excipient.

7. A set of at least two nucleic acids encoding the recombinant replication-defective adenoviral vector of claim 1, wherein two nucleic acids within said set are capable of a homologous recombination event among said two nucleic acids, which event leads to a nucleic acid competent to produce a recombinant replication-defective adenoviral vector.

8. A process for producing a recombinant replication-defective adenoviral vector, said process comprising:
expressing, in a packaging cell, a nucleic acid sequence, wherein said packaging cell complements all necessary elements for adenoviral replication that are absent from said nucleic acid sequence and wherein said nucleic acid sequence encodes a recombinant replication-defective adenoviral vector based on adenovirus serotype Ad49, having a gene of interest operatively linked to a promoter, and at least one adenovirus capsid protein of adenovirus serotype Ad11, Ad35 or Ad49; and
harvesting the resulting recombinant replication-defective adenoviral vector.

9. A process for producing a recombinant replication-defective adenoviral vector, said process comprising:
introducing into a packaging cell a nucleic acid sequence encoding said recombinant replication-defective adenoviral vector based on adenovirus serotype Ad49, but lacking a functional E1 region, said recombinant replication-defective adenoviral vector having a gene of interest operatively linked to a promoter and at least one adenovirus capsid protein of adenovirus serotype Ad11, Ad35 or Ad49;
culturing said packaging cell in a suitable medium, wherein said packaging cell expresses a nucleic acid sequence encoding an E1 protein that complements the E1 function lacking from said nucleic acid sequence introduced into said packaging cell;
complementing, by said packaging cell, activities necessary for adenoviral replication that are absent from said recombinant replication-defective adenoviral vector; and
harvesting the resulting recombinant replication-defective adenoviral vector.

10. A recombinant replication-defective adenoviral vector based on adenovirus serotype Ad49, said adenoviral vector comprising:
a gene of interest operatively linked to a promoter;
at least one adenovirus capsid protein from adenovirus serotype Ad11, Ad35 or Ad49; and
a genomic nucleic acid sequence encoding said recombinant replication-defective adenoviral vector.

11. The recombinant replication-defective adenoviral vector of claim 10, wherein said genomic nucleic acid sequence comprises an E4-Orf6 nucleic acid from an adenovirus of subgroup C.

12. The recombinant replication-defective adenoviral vector of claim 11, wherein the subgroup C adenovirus is Ad5.

13. The recombinant replication-defective adenoviral vector of claim 11, wherein the E4-Orf6 from the subgroup C adenovirus replaces the Ad49 E4-Orf6 nucleic acid sequence.

14. A set of at least two nucleic acid sequences encoding the recombinant replication-defective adenoviral vector of claim 11, wherein two nucleic acid sequences within said set are capable of a homologous recombination event among said two nucleic acid sequences, which event leads to a nucleic acid sequence competent to produce a recombinant replication-defective adenoviral vector.

15. The process of claim 8, wherein said nucleic acid sequence encoding said recombinant replication-defective adenoviral vector further comprises an E4-Orf6 nucleic acid sequence from an adenovirus of subgroup C.

16. The process of claim 15, wherein the subgroup C adenovirus is Ad5.

17. The process of claim 8, wherein said packaging cell is a PER.C6® cell.

18. The process of claim 9, wherein said nucleic acid sequence encoding said recombinant replication-defective adenoviral vector further comprises an E4-Orf6 nucleic acid sequence from an adenovirus of subgroup C.

19. The process of claim 18, wherein the subgroup C adenovirus is Ad5.

20. The process of claim 9, wherein said packaging cell is a PER.C6® cell.
